(19) United States
(12) Patent Application Publication
(10) Pub. No.: US 2024/0076639 A1
(43) Pub. Date: Mar. 7, 2024

(54) VARIANTS OF CAS12A NUCLEASES AND METHODS OF MAKING AND USE THEREOF

(71) Applicant: Pairwise Plants Services, Inc., Durham, NC (US)

(72) Inventor: Joseph Matthew Watts, Cary, NC (US)

(21) Appl. No.: 18/502,136

(22) Filed: Nov. 6, 2023

Related U.S. Application Data
(63) Continuation of application No. 17/071,095, filed on Oct. 15, 2020, now Pat. No. 11,866,745.
(60) Provisional application No. 62/916,392, filed on Oct. 17, 2019.

(51) Int. Cl. C12N 9/22; C07K 14/20; C12N 15/10; C12N 15/113

(52) U.S. Cl. CPC C12N 9/22; C07K 14/20; C12N 15/102; C12N 15/113; C07K 2319/70; C12N 2310/20

(57) ABSTRACT

This invention relates to variants of Cas12a nucleases having altered protospacer adjacent motif recognition specificity. The invention further relates to methods of making CRISPR-CAS nuclease variants and methods of modifying nucleic acids using the variants.

### STATEMENT OF PRIORITY

This application is a continuation application of U.S. patent application Ser. No. 17/071,095, filed on Oct. 15, 2020, which claims the benefit, under 35 U.S.C. § 119 (e), of U.S. Provisional Application No. 62/916,392 filed on Oct. 17, 2019, the entire contents of which is incorporated by reference herein.

### STATEMENT REGARDING ELECTRONIC FILING OF A SEQUENCE LISTING

A Sequence Listing in XML format, entitled 1499-7CT_ST26.xml, 123,645 bytes in size, generated on Oct. 30, 2023 and filed herewith, is hereby incorporated by reference into the specification for its disclosures.

### FIELD OF THE INVENTION

This invention relates to variants of Cas12a CRISPR-Cas nucleases having altered protospacer adjacent motif recognition specificity. The invention further relates to methods of making the CRISPR-CAS nuclease variants and methods of modifying nucleic acids using the variants.

### BACKGROUND OF THE INVENTION

Genome editing/modifying is a process that utilizes site-directed nucleases, for example, CRISPR-Cas nucleases, to introduce variation at a targeted genomic location. The most widely utilized nuclease for genome modification, Cas9, can introduce mutations at a genomic region upstream of an NGG motif (e.g., a protospacer adjacent motif (PAM)). Other Cas nucleases have different PAM recognition specificities. When the PAM specificities of these nucleases are particularly stringent, they can reduce the usefulness of the nuclease for genome modification by limiting the number of genomic target sites available for modification by that nuclease.

To address the shortcomings in the art, the present invention provides modified CRISPR-Cas nucleases having improved PAM specificity and methods for designing, identifying and selecting such CRISPR-Cas nucleases.

### SUMMARY OF THE INVENTION

One aspect of the invention provides a modified Lachnospiraceae bacterium CRISPR (Clustered Regularly Interspaced Short Palindromic Repeats) Cas12a (LbCas12a) polypeptide, wherein the modified LbCas12a polypeptide comprises, consists essentially of, or consists of an amino acid sequence having at least 80% identity to the amino acid sequence of SEQ ID NO:1 (LbCas12a) and a mutation at one or more (e.g., 1, 2, 3, 4, 5, 6, 7, 8, 9, 10, 11, 12, 13 or more) of the following positions of K116, K120, K121, D122, E125, T148, T149, T152, D156, E159, Q529, G532, D535, K538, D541, Y542, L585, K591, M592, K595, V596, S599, K600, K601, Y616, Y646, and/or W649 with reference to the position numbering of SEQ ID NO:1 in any combination, optionally a mutation at one or more of the following positions of K116, K120, K121, D122, E125, T152, D156, E159, G532, D535, K538, D541, and/or K595 with reference to the position numbering of SEQ ID NO:1 in any combination.

A second aspect of the invention provides a Type V Clustered Regularly Interspaced Short Palindromic Repeats (CRISPR)—associated (Cas) (CRISPR-Cas) system comprising: (a) a fusion protein comprising (i) a modified LbCas12a polypeptide of the invention or a nucleic acid encoding the modified LbCas12a polypeptide of the invention, and (ii) a polypeptide of interest or a nucleic acid encoding the polypeptide of interest; and (b) a guide nucleic acid (CRISPR RNA, CRISPR DNA, crRNA,crDNA) comprising a spacer sequence and a repeat sequence, wherein the guide nucleic acid is capable of forming a complex with the modified LbCas12a polypeptide or the fusion protein, and the spacer sequence is capable of hybridizing to a target nucleic acid, thereby guiding the modified LbCas12a polypeptide and the polypeptide of interest to the target nucleic acid, whereby the target nucleic acid is modified or modulated.

A third aspect of the invention provides a method of modifying a target nucleic acid, comprising: contacting the target nucleic acid with: (a)(i) a modified LbCas12a polypeptide of the invention, or a fusion protein comprising a modified LbCas12a polypeptide of the invention, and (ii) a guide nucleic acid (e.g., CRISPR RNA, CRISPR DNA, crRNA, crDNA); (b) a complex comprising the modified LbCas12a polypeptide of the invention and a guide nucleic acid; (c) a composition comprising (i) a modified lbCas12a polypeptide of the invention, or a fusion protein of the invention, and (ii) a guide nucleic acid; and/or (d) a system of the invention, thereby modifying the target nucleic acid.

A fourth aspect of the invention provides a method of modifying a target nucleic acid, comprising contacting a cell or a cell free system comprising the target nucleic acid with: (a)(i) a polynucleotide encoding a modified LbCas12a polypeptide of the invention, or an expression cassette or vector comprising the same, and (ii) a guide nucleic acid, or an expression cassette or vector comprising the same; and/or (b) a nucleic acid construct encoding a (i) complex comprising a modified LbCas12a polypeptide or a fusion protein of the invention, and (ii) a guide nucleic acid, or an expression cassette or vector comprising the same, thereby modifying the target nucleic acid.

A fifth aspect of the invention provides a method of editing a target nucleic acid, comprising: contacting the target nucleic acid with: (a)(i) a fusion protein comprising a modified LbCas12a polypeptide of the invention and (a)(ii) a guide nucleic acid; (b) a complex comprising the fusion protein of the invention, and a guide nucleic acid; (c) a composition comprising the fusion protein of the invention and a guide nucleic acid; and/or, (d) a system of the invention, thereby editing the target nucleic acid.

A sixth aspect of the invention provides a method of editing a target nucleic acid, comprising contacting a cell or a cell free system comprising the target nucleic acid with: (a)(i) a polynucleotide encoding a fusion protein comprising a modified LbCas12a polypeptide of the invention, or an expression cassette or vector comprising the same, and (a)(ii) a guide nucleic acid, or an expression cassette or vector comprising the same; and/or (b) a nucleic acid construct encoding a complex comprising a fusion protein comprising a modified LbCas12a polypeptide of the invention, and a guide nucleic acid, or an expression cassette or vector comprising the same; and/or (c) a system of the invention, thereby editing the target nucleic acid.

A seventh aspect of the invention provides a method of constructing a randomized DNA library comprising double stranded nucleic acid molecules for determining protospacer adjacent motif (PAM) requirements/specificity of a CRISPR-Cas nuclease having a PAM recognition site at the 5′ end of the protospacer, the method comprising: preparing two or more double stranded nucleic acid molecules comprising the following steps: (a) synthesizing a non-target oligonucleotide (first) strand and a target oligonucleotide (second) strand for each of the two or more double stranded nucleic acid molecules, wherein the non-target oligonucleotide strand comprises, 5′ to 3′: (i) a first sequence having about 5 to about 15 nucleotides, (ii) a second sequence having at least four randomized nucleotides, (iii) a protospacer sequence comprising about 16 to about 25 nucleotides, and (iv) a third sequence having about 5 to about 20 nucleotides, wherein the first sequence having about 5 to 15 nucleotides of (i) is immediately adjacent to the 5′ end of the second sequence of (ii), the second sequence of (ii) is immediately adjacent to the 5′ end of the protospacer sequence of (iii), and the protospacer sequence is immediately adjacent to the 5′ end of the third sequence of (iv); and the target oligonucleotide (second) strand complementary to the non-target oligonucleotide strand; and (b) annealing the non-target oligonucleotide strand to the complementary target oligonucleotide strand to produce a double stranded nucleic acid molecule, wherein the first sequence comprises a restriction site (at its 5′ end) and the third sequence comprises a restriction site (at its 3′ end), wherein the first sequence (i), the protospacer sequence (iii) and the third sequence (iv) of each of the two or more double stranded nucleic acid molecules is identical, thereby constructing the randomized DNA library comprising double stranded nucleic acid molecules.

An eighth aspect of the invention provides a method of constructing a randomized DNA library comprising double stranded nucleic acid molecules for determining protospacer adjacent motif (PAM) requirements/specificity of a CRISPR-Cas nuclease having a PAM recognition site at the 3′ end of the protospacer, the method comprising: preparing two or more double stranded nucleic acid molecules comprising the following steps: (a) synthesizing a non-target oligonucleotide (first) strand and a target oligonucleotide (second) strand for each of the two or more double stranded nucleic acid molecules, wherein the non-target oligonucleotide strand comprises, 5′ to 3′: (i) a first sequence having about 5 to about 20 nucleotides, (ii) a protospacer sequence comprising about 16 to about 25 nucleotides, (iii) a second sequence having at least four randomized nucleotides, and (iv) a third sequence having about 5 to about 15 nucleotides, wherein the first sequence having about 5 to 20 nucleotides of (i) is immediately adjacent to the 5′ end of the protospacer sequence of (ii), the second sequence of (iii) is immediately adjacent to the 3′ end of the protospacer sequence of (iii), and the third sequence of (iv) is immediately adjacent to the 3′ end of the second sequence of (iii); and the target oligonucleotide (second) strand is complementary to the non-target oligonucleotide strand; and (b) annealing the non-target oligonucleotide strand to the complementary target oligonucleotide strand to produce a double stranded nucleic acid molecule, wherein the first sequence (i) comprises a restriction site (at its 5′ end) and the third sequence (iv) comprises a restriction site (at its 3′ end), wherein the first sequence (i), the protospacer sequence (ii) and the third sequence (iv) of each of the two or more double stranded nucleic acid molecules is identical, thereby constructing the randomized DNA library comprising double stranded nucleic acid molecules.

A ninth aspect of the invention provides a randomized DNA library for determining protospacer adjacent motif (PAM) requirements/specificity of a CRISPR-Cas nuclease having a PAM recognition site on the 5′ end of protospacer, the randomized DNA library comprising two or more double stranded nucleic acid molecules each of which comprises: (a) a non-target oligonucleotide (first) strand and a target oligonucleotide (second) strand, wherein the non-target oligonucleotide strand comprises, 5′ to 3′: (i) a first sequence having about 5 to about 15 nucleotides (e.g., about 5, 6, 7, 8, 9, 10, 11, 12, 13, 14, or 15 nucleotides, and any range or value therein), (ii) a second sequence having at least four randomized nucleotides (e.g., at least 4, 5, 6, 7, 8, 9, 10, or more, and any range or value therein), (iii) a protospacer sequence comprising about 16 to about 25 nucleotides e.g., about 16, 17, 18, 19, 20, 21, 22, 23, 24, or 25 nucleotides, and (iv) a third sequence having about 5 to about 20 nucleotides (e.g., about 5, 6, 7, 8, 9, 10, 11, 12, 13, 14, 15, 16, 17, 18, 19, 20 nucleotides, and any range or value therein), wherein the first sequence having about 5 to 15 nucleotides of (i) is immediately adjacent to the 5′ end of the second sequence of (ii), the second sequence of (ii) is immediately adjacent to the 5′ end of the protospacer sequence of (iii), and the protospacer sequence is immediately adjacent to the 5′ end of the third sequence of (iv); and the target oligonucleotide (second) strand is complementary to the non-target oligonucleotide strand; and (b) the non-target oligonucleotide strand is annealed to the complementary target oligonucleotide strand to produce a double stranded nucleic acid molecule, wherein the first sequence comprises a restriction site (at its 5′ end) and the third sequence comprises a restriction site (at its 3′ end), wherein the first sequence (i), the protospacer sequence (iii) and the third sequence (iv) of each of the two or more double stranded nucleic acid molecules are identical.

A tenth aspect of the invention provides a randomized DNA library for determining protospacer adjacent motif (PAM) requirements/specificity of a CRISPR-Cas nuclease having a PAM recognition site on the 3′ end of protospacer, the randomized DNA library comprising two or more double stranded nucleic acid molecules each of which comprises: (a) a non-target oligonucleotide (first) strand and a target oligonucleotide (second) strand, wherein the non-target oligonucleotide strand comprises, 5′ to 3′: (i) a first sequence having about 5 to about 20 nucleotides (e.g., about 5, 6, 7, 8, 9, 10, 11, 12, 13, 14, 15, 16, 17, 18, 19, 20 nucleotides, and any range or value therein), (ii) a protospacer sequence comprising about 16 to about 25 nucleotides e.g., about 16, 17, 18, 19, 20, 21, 22, 23, 24, or 25 nucleotides, (iii) a second sequence having at least four randomized nucleotides (e.g., at least 4, 5, 6, 7, 8, 9, 10, or more, and any range or value therein), and (iv) a third sequence having about 5 to about 15 nucleotides (e.g., about 5, 6, 7, 8, 9, 10, 11, 12, 13, 14, 15 nucleotides, and any range or value therein), wherein the first sequence having about 5 to 20 nucleotides of (i) is immediately adjacent to the 5′ end of the protospacer sequence of (ii), the second sequence of (iii) is immediately adjacent to the 3′ end of the protospacer sequence of (iii), and the third sequence of (iv) is immediately adjacent to the 3′ end of the second sequence of (iii); and the target oligonucleotide (second) strand is complementary to the non-target oligonucleotide strand; and (b) the non-target oligonucleotide strand is annealed to the complementary target oligonucleotide strand to produce a double stranded nucleic acid molecule, wherein the first sequence comprises a restriction site (at its 5′ end) and the third sequence comprises a restriction site (at its 3′ end), wherein the first sequence (i), the protospacer sequence (ii) and the third sequence (iv) of each of the two or more double stranded nucleic acid molecules are identical.

The invention further provides expression cassettes and/or vectors comprising polynucleotides encoding CRISPR-Cas nucleases and/or fusion proteins of the invention and/or cells comprising polynucleotides, polypeptides and/or fusion proteins of the invention and/or kits comprising the same.

These and other aspects of the invention are set forth in more detail in the description of the invention below.

### BRIEF DESCRIPTION OF THE SEQUENCES

SEQ ID NOs:1-17, 49, 50 and 51 are exemplary nucleotide sequences encoding Cas12a nucleases.

SEQ ID NOs:18-22 are exemplary adenosine deaminases.

SEQ ID NOs:23-25 and SEQ ID NOs:42-48 are exemplary cytosine deaminases. SEQ ID NO:26 an exemplary nucleotide sequence encoding a uracil-DNA glycosylase inhibitor (UGI).

SEQ ID NOs: 27-29 provides an example of a protospacer adjacent motif position for a Type V CRISPR-Cas12a nuclease.

SEQ ID NOs:30-39 shows example nucleotide sequences useful for generating a randomized library of this invention for use in, for example, in vitro cleavage assays.

SEQ ID NOs:40-41 are exemplary regulatory sequences encoding a promoter and intron.

SEQ ID NO:52 provides the nucleotide sequence of an example expression cassette.

SEQ ID NO:53 provides the nucleotide sequence of an example vector.

SEQ ID NOs:54-61 provide example spacer sequences.

SEQ ID NO:62 provides an example CRISPR RNA.

### BRIEF DESCRIPTION OF THE DRAWINGS

FIG. 1 shows a diagram of an example PAM Library preparation of the invention. In this method, 5′ phosphorylated oligonucleotides are annealed and cloned into an EcoRI and SphI digested pUC19 vector. Sca I is used to linearize the vector (AGTACT sequence is not recognized by Lbcpf1). Top strand (SEQ ID NO:32); bottom strand (SEQ ID NO:33).

FIG. 2 shows mean protospacer adjacent motifs (PAMs) per gene for corn (top panel) and soy (bottom panel) within coding sequences. LbCpf1 genes can access far less gene sequences than Cas9 variants.

FIG. 3 shows mean accessible cytosines (panels A, B) and adenines (panels C, D) limited by PAMs in corn (panels A, C) and soy (panels B, D). As shown, LbCpf1 cytosines and adenines can access far less cytosines and adenines than Cas9 variants.

FIG. 4 provides a diagram showing a simplified PAM Determination Assay of Gao et al. (Nat Biotechnol 35(8):789-792 (2017)). Amplified fragments represent sequences that were not cut by the CRISPR-Cas nuclease, whereas fragments that are not amplified were cut by the CRISPR-Cas nuclease. Sequencing and comparison to an enzyme-free control identifies the nucleic acid sequences that were not amplified (i.e., that are present in the control population but not in the edited population) and thus, identifies the sequences that are recognized and cut by the nuclease. Top panel: top sequence (SEQ ID NO:36), middle sequence (SEQ ID NO:37), bottom sequence (SEQ ID NO:38); middle panel: top sequence (SEQ ID NO:39), bottom sequence (SEQ ID NO:37); bottom panel: top sequence (SEQ ID NO:36), bottom sequence (SEQ ID NO: 37).

FIG. 5. Average of 3 Illumina MiSeq NGS reads of the PAMDA library plotted highest to lowest number of counts. The 1024 library members, containing NNNNN, follow a normal distribution with an average of 39 reads.

FIG. 6. Cell sorting results of a negative control containing wtLbCas12a and a crRNA which did not target the plasmid spacers.

FIG. 7. Cell sorting results of wtLbCas12a and a crRNA targeting the plasmid spacers.

FIG. 8. Cell sorting results of LbCas12a-K595Y and a crRNA targeting the plasmid spacers.

FIG. 9. Cell sorting results of LbCas12a-G532R-K595R double mutation control and a crRNA targeting the plasmid spacers.

FIG. 10. Cell sorting results of LbCas12a-T152R-K595Y double mutation, a combination of two of the point mutations in this study, with a crRNA targeting the plasmid spacers.

FIG. 11. Cell sorting results of LbCas12a-T152R-K538W-K595Y triple mutation, a combination of three of the point mutations with a crRNA targeting the plasmid spacers.

FIG. 12. Total normalized NGS counts for two separate no crRNA controls and wildtype dLbCas12a and the reporter library.

FIG. 13. Single point mutation normalized PAM-SCANR scores for each of the 256 four nucleotide PAMs. The line through the graph shows the highest observed score for either of the two negative controls, 1.67.

FIG. 14. Combinatorial mutations normalized PAM-SCANR scores for each of the 256 four nucleotide PAMs.

FIG. 15 shows that combining mutants K538W and K595Y results in an enzyme LbCas12a-K538W-K595Y with unique PAM recognition sequences. In some cases, shared PAM recognition motifs from K538W (vertical hatched or K595Y (horizontal hatched) are recognized by the combination mutant, but often the combination results in completely novel PAM recognition sequences (thatched).

FIG. 16 shows combining multiple expanded PAM mutations can generate sometimes additive but often unique PAM recognition sequences

FIG. 17 compares all non-TTTV PAMs which showed above a 1.67 score from PAN-SCANR (grey boxes) to K595Y (left) and T152R (right). All but one of the PAM-SCANR positive PAMs above the 1.67 cutoff had PAM depletion scores above the 9.2 cutoff in vitro.

FIG. 18 shows percentage indel formed in HEK293T cells for each TTTV-containing spacer. Individual indel percentages are shown as circles, squares, or triangles for TTTC, TTTA, and TTTG, respectively. An average line and the value rounded to the nearest whole number is also shown for each spacer.

FIG. 19 shows LbCas12a_K595Y HEK293T maximum observed INDEL percentages per PAM tested. Values above 0.1% are outside of the noise of sequencing and represent authentic INDELs.

FIG. 20 shows LbCas12a_T152R HEK293T maximum observed INDEL percentages per PAM tested. Values above 0.1% are outside of the noise of sequencing and represent authentic INDELs.

FIG. 21 shows LbCas12a_K538W HEK293T maximum observed INDEL percentages per PAM tested. Values above 0.1% are outside of the noise of sequencing and represent authentic INDELs.

FIGS. 22A-22B. Linear correlation between % INDEL (max) and the normalized bacterial PAM-SCANR score for LbCas12a-T152R (FIG. 22A) and LbCas12a-K595Y (FIG. 22B).

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount.

A “heterologous” or a “recombinant” nucleotide sequence is a nucleotide sequence not naturally associated with a host cell into which it is introduced, including non-naturally occurring multiple copies of a naturally occurring nucleotide sequence.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the organism. A “homologous” nucleic acid sequence is a nucleotide sequence naturally associated with a host cell into which it is introduced.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement” as used herein can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

A “portion” or “fragment” of a nucleotide sequence of the invention will be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising, consisting essentially of and/or consisting of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the invention may be, where appropriate, included in a larger polynucleotide of which it is a constituent. As an example, a repeat sequence of guide nucleic acid of this invention may comprise a portion of a wild type CRISPR-Cas repeat sequence (e.g., a wild type Cas9 repeat, wild type Cas12a repeat, and the like).

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

Any nucleotide sequence, polynucleotide and/or recombinant nucleic acid construct of this invention can be codon optimized for expression in any organism of interest. Codon optimization is well known in the art and involves modification of a nucleotide sequence for codon usage bias using species specific codon usage tables. The codon usage tables are generated based on a sequence analysis of the most highly expressed genes for the organism/species of interest. When the nucleotide sequences are to be expressed in the nucleus, the codon usage tables are generated based on a sequence analysis of highly expressed nuclear genes for the species of interest. The modifications of the nucleotide sequences are determined by comparing the species specific codon usage table with the codons present in the native polynucleotide sequences. As is understood in the art, codon optimization of a nucleotide sequence results in a nucleotide sequence having less than 100% identity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.9%, and any range or value therein) to the native nucleotide sequence but which still encodes a polypeptide having the same function as that encoded by the original, native nucleotide sequence. Thus, in some embodiments of the invention, the polynucleotides, nucleic acid constructs, expression cassettes, and/or vectors of the invention (e.g., comprising/encoding a polypeptide, fusion protein, complex of the invention, e.g., a modified CRISPR-Cas nuclease) are codon optimized for expression in a particular species of interest, e.g., a particular plant species, a particular bacterial species, a particular animal species, and the like. In some embodiments, the codon optimized nucleic acid constructs, polynucleotides, expression cassettes, and/or vectors of the invention have about 70% to about 99.9% (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the polynucleotides, nucleic acid constructs, expression cassettes, and/or vectors of the invention that have not been codon optimized

In any of the embodiments described herein, a polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron).

By “operably linked” or “operably associated” as used herein in reference to polynucleotides, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus or the C-terminus) directly (e.g., via a peptide bond) or through a linker.

The term “linker” is art-recognized and refers to a chemical group, or a molecule linking two molecules or moieties, e.g., two domains of a fusion protein, such as, for example, a LbCas12a CRISPR-Cas nuclease domain and a polypeptide of interest (e.g., a nucleic acid-editing domain, a deaminase domain, an adenosine deaminase, a cytosine deaminase). A linker may be comprised of a single linking molecule or may comprise more than one linking molecule. In some embodiments, the linker can be an organic molecule, group, polymer, or chemical moiety such as a bivalent organic moiety. In some embodiments, the linker may be an amino acid or a peptide. In some embodiments, a peptide linker may be about 4 to about 100 or more amino acids in length, for example, about 4, 6, 7, 8, 9, 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. In some embodiments, a peptide linker may be a GS linker. A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227). In some embodiments, a promoter region may comprise at least one intron (e.g., SEQ ID NO:40 or SEQ ID NO:41).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)).

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9(5):297-306 (2015)), ZmSTK2_USP from maize (Wang et al. Genome 60(6):485-495 (2017)), LAT52 and LAT59 from tomato (Twell et al. Development 109(3):705-713 (1990)), Zm13 (U.S. Pat. No. 10,421,972), PLA₂-δ promoter from arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHES) (Kim et al, The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-30 1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron.

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof. As a non-limiting example, a nucleic acid construct of the present invention may encode a base editor comprising an optimized CRISPR-Cas nuclease (e.g., SEQ ID NOs:1-11 or 23-25) and a deaminase, wherein the nucleic acid construct further comprises a promoter comprising/associated with an intron. As a further non-limiting example, a nucleic acid construct of the present invention may encode a base editor comprising an optimized CRISPR-Cas nuclease (e.g., SEQ ID NOs:1-11 or 23-25) and a deaminase, wherein the nuclease and/or the deaminase comprises one or more introns and optionally, the nucleic acid construct further comprises a promoter comprising/associated with an intron.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a nucleic acid construct of the invention (e.g., encoding a modified LbCas12a of the invention), wherein the nucleic acid construct is operably associated with at least a control sequence (e.g., a promoter). Thus, some embodiments of the invention provide expression cassettes designed to express, for example, a nucleic acid construct of the invention (e.g., a nucleic acid construct of the invention encoding a modified LbCas12a of the invention).

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. The termination region and/or the enhancer region may be native to the transcriptional initiation region, may be native to a gene encoding, e.g., a LbCas12a nuclease encoded by a nucleic acid construct of the invention, may be native to a host cell, or may be native to another source (e.g., foreign or heterologous to the promoter, to a gene encoding a LbCas12a nuclease encoded by a nucleic acid construct of the invention, to a host cell, or any combination thereof). The enhancer region may be native to a gene encoding a LbCas12a nuclease encoded by a nucleic acid construct of the invention, may be native to a host cell, or may be from another source (e.g., foreign or heterologous to the promoter, to the gene encoding the LbCas12a nuclease encoded by a nucleic acid construct of the invention, to the host cell, or any combination thereof).

An expression cassette of the invention also can include a nucleotide sequence encoding a selectable marker, which can be used to select a transformed host cell. As used herein, “selectable marker” means a nucleotide sequence that when expressed imparts a distinct phenotype to the host cell expressing the marker and thus allows such transformed cells to be distinguished from those that do not have the marker. Such a nucleotide sequence may encode either a selectable or screenable marker, depending on whether the marker confers a trait that can be selected for by chemical means, such as by using a selective agent (e.g., an antibiotic and the like), or whether the marker is simply a trait that one can identify through observation or testing, such as by screening (e.g., fluorescence). Many examples of suitable selectable markers are known in the art and can be used in the expression cassettes described herein.

In addition to expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid construct of this invention and/or expression cassettes comprising the same may be comprised in vectors as described herein and as known in the art. In some embodiments, the vector may be a high copy number vector (e.g., a high copy number E. coli vector; e.g., pUC, pBluescript, pGEM and the like). Thus, for example, a library of the present invention may be constructed using a high copy number vector.

As used herein, “contact”, “contacting”, “contacted,” and grammatical variations thereof, refers to placing the components of a desired reaction together under conditions suitable for carrying out the desired reaction (e.g., transformation, transcriptional control, genome editing, nicking, and/or cleavage). Thus, for example, a target nucleic acid may be contacted with (a) a polynucleotide and/or nucleic acid construct of the invention encoding a modified LbCas12a nuclease of the invention and (b) a guide nucleic acid, under conditions whereby the polynucleotide/nucleic acid construct is expressed and the modified LbCas12a nuclease is produced, wherein the nuclease forms a complex with the guide nucleic acid and the complex hybridizes to the target nucleic acid, thereby modifying the target nucleic acid. In some embodiments, a target nucleic acid may be contacted with (a) a modified LbCas12a nuclease of the invention and/or a fusion protein comprising the same (e.g., the modified LbCas12a nuclease of the invention and a polypeptide of interest (e.g., a deaminase)) and (b) a guide nucleic acid, wherein the modified LbCas12a nuclease forms a complex with the guide nucleic acid and the complex hybridizes to the target nucleic acid, thereby modifying the target nucleic acid. As described herein, the target nucleic acid may be contacted with the polynucleotides/nucleic acid constructs/polypeptides of the invention prior to, concurrently with, or after contact with the guide nucleic acid.

As used herein, “modifying” or “modification” in reference to a target nucleic acid includes editing (e.g., mutating), covalent modification, exchanging/substituting nucleic acids/nucleotide bases, deleting, cleaving, nicking, and/or transcriptional control of a target nucleic acid.

“Introducing,” “introduce,” “introduced” (and grammatical variations thereof) in the context of a polynucleotide of interest means presenting a nucleotide sequence of interest (e.g., polynucleotide, a nucleic acid construct, and/or a guide nucleic acid) to a host organism or cell of said organism (e.g., host cell; e.g., a plant cell) in such a manner that the nucleotide sequence gains access to the interior of a cell. Thus, for example, a polynucleotide of the invention encoding a modified LbCas12a nuclease as described herein and guide nucleic acid may be introduced into a cell of an organism, thereby transforming the cell with modified LbCas12a nuclease and guide nucleic acid.

The term “transformation” as used herein refers to the introduction of a heterologous nucleic acid into a cell. Transformation of a cell may be stable or transient. Thus, in some embodiments, a host cell or host organism may be stably transformed with a polynucleotide/nucleic acid molecule of the invention. In some embodiments, a host cell or host organism may be transiently transformed with a polynucleotide/nucleic acid construct of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Accordingly, in some embodiments, nucleotide sequences, polynucleotides, and/or nucleic acid constructs of the invention and/or expression cassettes and/or vectors comprising the same may be expressed transiently and/or they can be stably incorporated into the genome of the host organism. Thus, in some embodiments, a nucleic acid construct of the invention (e.g., encoding a modified LbCas12a nuclease of the invention, or a fusion protein thereof; e.g., a fusion protein comprising the modified LbCas12a nuclease linked to e.g., a polynucleotide of interest, e.g., a deaminase domain), wherein the nucleic acid construct encoding the modified LbCas12a nuclease is codon optimized for expression in an organism (e.g., a plant, a mammal, a fungus, a bacterium, and the like) may be transiently introduced into a cell of the organism along with a guide nucleic acid and as such no DNA maintained in the cell.

A polynucleotide/nucleic acid construct of the invention can be introduced into a cell by any method known to those of skill in the art. In some embodiments of the invention, transformation of a cell comprises nuclear transformation. In other embodiments, transformation of a cell comprises plastid transformation (e.g., chloroplast transformation). In still further embodiments, a polynucleotide/nucleic acid construct of the invention can be introduced into a cell via conventional breeding techniques.

Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)).

A nucleotide sequence therefore can be introduced into a host organism or its cell in any number of ways that are well known in the art. The methods of the invention do not depend on a particular method for introducing one or more nucleotide sequences into the organism, only that they gain access to the interior of at least one cell of the organism. Where more than one nucleotide sequence is to be introduced, they can be assembled as part of a single nucleic acid construct, or as separate nucleic acid constructs, and can be located on the same or different nucleic acid constructs. Accordingly, the nucleotide sequences can be introduced into the cell of interest in a single transformation event, and/or in separate transformation events, or, alternatively, where relevant, a nucleotide sequence may be incorporated into a plant, for example, as part of a breeding protocol.

The present invention is directed to Cas12a nucleases modified to include non-natural PAM recognition sites/sequences (e.g., a Cas12a nuclease that comprises non-natural PAM recognition specificity in addition to or instead of the natural PAM recognition specificity for that particular Cas12a nuclease). In addition, the present invention is directed to methods for designing, identifying and selecting Cas12a nucleases having desirable characteristics including improved PAM recognition specificity.

As used herein, in reference to a modified Cas12a polypeptide, “altered PAM specificity” means that the PAM specificity of the nuclease is altered from that of the wild type nuclease (e.g., non-native PAM sequences are recognized in addition to and/or instead of the native PAM sequence. For example, a modified Cas12a nuclease would be altered in its PAM specificity if it recognizes a PAM sequence other than and/or in addition to the native Cas12 a PAM sequence of TTTV, wherein V is A, C or G.

The present invention is directed to LbCas12a nucleases having modified PAM recognition specificities. In some embodiments, the present invention provides a modified Lachnospiraceae bacterium CRISPR (Clustered Regularly Interspaced Short Palindromic Repeats) Cas12a (LbCas12a) polypeptide, wherein the modified LbCas12a polypeptide comprises an amino acid sequence having at least 80% identity (e.g., about 80, 81, 82, 83, 84, 85, 86, 87, 88, 89, 90, 91, 92, 93, 94, 95, 96, 97, 98, 99, 100% identity; e.g., about 80% to about 100%, about 85% to about 100%, about 90% to about 100% about 95% to about 100%) to the amino acid sequence of SEQ ID NO:1 (LbCas12a) and a mutation at one or more than one (e.g., 2, 3, 4, 5, 6, 7, 8, 9, 10, 11, 12, 13 or more) of the following positions of K116, K120, K121, D122, E125, T148, T149, T152, D156, E159, Q529, G532, D535, K538, D541, Y542, L585, K591, M592, K595, V596, S599, K600, K601, Y616, Y646, and/or W649 with reference to the position numbering of SEQ ID NO:1, optionally a mutation at one or more than one of the following positions of K116, K120, K121, D122, E125, T152, D156, E159, G532, D535, K538, D541, and/or K595 with reference to the position numbering of SEQ ID NO:1. In some embodiments, a mutation of a Cas12a (LbCas12a) polypeptide comprises, consists essentially of, or consists of a mutation at one or more than one of the following positions of K116, K120, K121, D122, E125, T152, D156, E159, G532, D535, K538, D541, and/or K595 with reference to the position numbering of SEQ ID NO:1 in any combination. Thus, a modified LbCas12a polypeptide of this invention may comprise a single mutation at any one of the positions of K116, K120, K121, D122, E125, T148, T149, T152, D156, E159, Q529, G532, D535, K538, D541, Y542, L585, K591, M592, K595, V596, S599, K600, K601, Y616, Y646, and/or W649 with reference to the position numbering of SEQ ID NO:1 or may comprise a combination of mutations any two or more positions of K116, K120, K121, D122, E125, T148, T149, T152, D156, E159, Q529, G532, D535, K538, D541, Y542, L585, K591, M592, K595, V596, S599, K600, K601, Y616, Y646, and/or W649 with reference to the position numbering of SEQ ID NO:1.

In some embodiments, a mutation of a Cas12a (LbCas12a) polypeptide comprises, consists essentially of or consists of one or more than one of the following mutations of K116N, K116R, K120H, K120N, K120Q, K120R, K120T, K121D, K121G, K121H, K121Q, K121R, K121S, K121T, D122H, D122K, D122N, D122R, E125K, E125Q, E125R, E125Y, T148A, T148C, T148H, T148S, T149C, T149F, T149G, T149H, T149N, T149P, T149S, T149V, T152E, T152F, T152H, T152K, T152L, T152Q, T152R, T152W, T152Y, D156E, D156H, D156I, D156K, D156L, D156Q, D156R, D156W, D156Y, E159K, E159Q, E159R, E159Y, Q529A, Q529D, Q529F, Q529G, Q529H, Q529N, Q529P, Q529S, Q529T, Q529W, G532A, G532C, G532D, G532F, G532H, G532K, G532L, G532N, G532Q, G532S, D535A, D535H, D535K, D535N, D535S, D535T, D535V, K538C, K538F, K538G, K538H, K538L, K538M, K538Q, K538R, K538V, K538W, K538Y, D541A, D541E, D541H, D541I, D541N, D541R, D541Y, Y542F, Y542H, Y542K, Y542L, Y542M, Y542N, Y542R, Y542T, Y542V, L585F, L585G, L585H, K591A, K591F, K591G, K591H, K591R, K591S, K591W, K591Y, M592A, M592E, M592Q, K595H, K595L, K595M, K595Q, K595R, K595S, K595W, K595Y, V596H, V596T, S599G, S599H, S599N,K600G, K600H, K600R, K601H, K601Q, K601R, K601T, Y616E, Y616F, Y616H, Y616K, Y616R, Y646E, Y646H, Y646K, Y646N, Y646Q, Y646R, Y646W, W649H, W649K, W649R, W649S and/or W649Y with reference to the position numbering of SEQ ID NO:1. As would be understood, any single Cas12a polypeptide having two or more mutations would comprise only a single mutation at any given position. Thus, for example, a polypeptide may have mutation at position D535 of any one of D535A, D535H, D535K, D535N, D535S, D535T, or D535V, but the same polypeptide may further comprise a mutation at one or more than one of any of the other positions as described herein. In some embodiments, a mutation of a Cas12a (LbCas12a) polypeptide comprises, consists essentially of or consists of one or more than one mutation of K116N, K116R, K120H, K120N, K120Q, K120R, K120T, K121D, K121G, K121H, K121Q, K121R, K121S, K121T, D122H, D122K, D122N, D122R, E125K, E125Q, E125R, E125Y, T152E, T152F, T152H, T152K, T152L, T152Q, T152R, T152W, T152Y, D156E, D156H, D156I, D156K, D156L, D156Q, D156R, D156W, D156Y, E159K, E159Q, E159R, E159Y, G532A, G532C, G532D, G532F, G532H, G532K, G532L, G532N, G532Q, G532S, D535A, D535H, D535K, D535N, D535S, D535T, D535V, K538C, K538F, K538G, K538H, K538L, K538M, K538Q, K538R, K538V, K538W, K538Y, D541A, D541E, D541H, D541I, D541N, D541R, D541Y, K595H, K595L, K595M, K595Q, K595R, K595S, K595W, and/or K595Y with reference to the residue position numbering of SEQ ID NO:1 in any combination. In some embodiments, a mutation of a Cas12a (LbCas12a) polypeptide comprises, consists essentially of or consists of one or more than one mutation of K116R, K116N, K120Y, K121S, K121R, D122H, D122N, E125K, T152R, T152K, T152Y, T152Q, T152E, T152F, D156R, D156W, D156Q, D156H, D1561, D156V, D156L, D156E, E159K, E159R, G532N, G532S, G532H, G532K, G532R, G532L, D535N, D535H, D535T, D535,S D535A, D535W, K538R K538V, K538Q, K538W, K538Y, K538F, K538H, K538L, K538M, K538C, K538G, K538A, D541E, K595R, K595Q, K595Y, K595W, K595H, K595S, and/or K595M with reference to the position numbering of SEQ ID NO:1. As would be understood, any single Cas12a polypeptide having two or more mutations would comprise a single mutation at any given position. Thus, for example, a polypeptide may have a mutation at position D535 of any one of D535A, D535H, D535K, D535N, D535S, D535T, or D535V, and may further comprise a mutation at one or more than one at any other position as described herein.

In some embodiments, the mutation does not comprise, consist essentially of or consist of a mutation of D156R, G532R, K538R, K538V, Y542R or K595R with reference to position numbering of SEQ ID NO:1. In some embodiments, the mutation of the Cas12a (LbCas12a) polypeptide does not comprise, consist essentially of or consist of the combination of mutations of G532R and K595R, of G532R, K538V and Y542R, or of D156R, G532R and K532R with reference to position numbering of SEQ ID NO:1.

In some embodiments, the modified LbCas12a polypeptide may comprise one or more amino acid mutations of SEQ ID NO:1 as set forth in Table 2 (in Example 2).

In some embodiments, the modified LbCas12a polypeptide may comprise an altered protospacer adjacent motif (PAM) specificity as compared to wild type LbCas12a (e.g., SEQ ID NO:1). A modified LbCas12a polypeptide of the present invention may comprise an altered PAM specificity, wherein the altered PAM specificity includes, but is not limited to, NNNG, NNNT, NNNA, NNNC, NNG, NNT, NNC, NNA, NG, NT, NC, NA, NN, NNN, NNNN, wherein each N of each sequence is independently selected from any of T, C, G, or A. In some embodiments, the altered PAM specificity may include, but is not limited to, TTTA, TTTC, TTTG, TTTT, TTCA, TTCC, TTCG, TTCT, ATTC, CTTA, CTTC, CTTG, GTTC, TATA, TATC, CTCC, TCCG, TACA, TCCG, TACA, TCCG, TCCC, TCCA, and/or TATG. In some embodiments, the altered PAM specificity may be NNNN, wherein each N of each sequence is independently selected from any of T, C, G, or A.

In addition to having an altered PAM recognition specificity a modified LbCas12a nuclease may further comprise a mutation in the nuclease active site (e.g., RuvC domain) (e.g., deadLbCas12a, dLbCas12a). Such modifications may result in the LbCas12a polypeptide having reduced nuclease activity (e.g., nickase activity) or no nuclease activity.

In some embodiments, a Type V Clustered Regularly Interspaced Short Palindromic Repeats (CRISPR)—associated (Cas) (CRISPR-Cas) system is provided, the system comprising: (a) a fusion protein comprising (i) a modified LbCas12a nuclease of the invention or a nucleic acid encoding the modified LbCas12a nuclease of the invention, and (ii) a polypeptide of interest or a nucleic acid encoding the polypeptide of interest; and (b) a guide nucleic acid (CRISPR RNA, CRISPR DNA, crRNA,crDNA) comprising a spacer sequence and a repeat sequence, wherein the guide nucleic acid is capable of forming a complex with the modified LbCas12a nuclease or the fusion protein and the spacer sequence is capable of hybridizing to a target nucleic acid, thereby guiding modified LbCas12a nuclease and the polypeptide of interest to the target nucleic acid, whereby the system is capable of modifying (e.g., cleaving or editing) or modulating (e.g., modulating transcription) the target nucleic acid. In some embodiments, the system comprises a polypeptide of interest linked to the C-terminus and/or the N-terminus of the modified LbCas12a nuclease (e.g., a fusion protein), optionally via a peptide linker.

Additionally, provided herein are fusion proteins that comprise a modified Cas12a nuclease of the invention. In some embodiments, the fusion protein may comprise a polypeptide of interest linked to the C-terminus and/or to the N-terminus of the modified LbCas12a. In some embodiments, the present invention provides a fusion protein comprising a modified LbCas12a and an optional intervening linker linking the polypeptide of interest.

Any linker known in the art or later identified that does not interfere with the activity of the fusion protein may be used. A linker that does not “interfere” with the activity of a fusion protein is a linker that does not reduce or eliminate the activity of the polypeptides of the fusion protein (e.g., the nuclease and/or the polypeptide of interest); that is, the nuclease activity, nucleic acid binding activity, editing activity, and/or any other activity of the nuclease or peptide of interest is maintained in a fusion protein in which the nuclease and the polypeptide of interest are tethered to one another via the linker. In some embodiments, a peptide linker may be linked (e.g., at its N-terminus) to the C-terminus of a modified LbCas12a, optionally wherein the fusion protein may further comprise a polypeptide of interest linked to the C-terminus of the linker. In some embodiments, a peptide linker may be linked (e.g., at its C-terminus) to the N-terminus of a modified LbCas12a, optionally wherein the fusion protein may further comprise a polypeptide of interest linked to the N-terminus of the linker. In some embodiments, a modified LbCas12a of the invention may be linked at both its C-terminus and N-terminus to a linker and/or a polypeptide of interest (directly or via a linker).

In some embodiments, a linker useful with the invention may be an amino acid or a peptide. In some embodiments, a peptide linker useful with this invention may be about 4 to about 100 or more amino acids in length, for example, about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 4 to about 40, about 4 to about 50, about 4 to about 60, about 5 to about 40, about 5 to about 50, 15 about 5 to about 60, about 9 to about 40, about 9 to about 50, about 9 to about 60, about 10 to about 40, about 10 to about 50, about 10 to about 60, or about 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length). In some embodiments, a peptide linker may be a GS linker.

A polypeptide of interest useful with this invention can include, but is not limited to, a polypeptide or protein domain having deaminase (deamination) activity, nickase activity, recombinase activity, transposase activity, methylase activity, glycosylase (DNA glycosylase) activity, glycosylase inhibitor activity (e.g., uracil-DNA glycosylase inhibitor (UGI)). demethylase activity, transcription activation activity, transcription repression activity, transcription release factor activity, histone modification activity, nuclease activity, single-strand RNA cleavage activity, double-strand RNA cleavage activity, restriction endonuclease activity (e.g., Fok1), nucleic acid binding activity, methyltransferase activity, DNA repair activity, DNA damage activity, dismutase activity, alkylation activity, depurination activity, oxidation activity, pyrimidine dimer forming activity, integrase activity, transposase activity, polymerase activity, ligase activity, helicase activity, and/or photolyase activity.

In some embodiments, a polypeptide of interest may comprise at least one polypeptide or protein domain having deaminase activity. In some embodiments, the at least one polypeptide or protein domain may be an adenine deaminase domain. An adenine deaminase (or adenosine deaminase) useful with this invention may be any known or later identified adenine deaminase from any organism (see, e.g., U.S. Pat. No. 10,113,163, which is incorporated by reference herein for its disclosure of adenine deaminases). An adenine deaminase can catalyze the hydrolytic deamination of adenine or adenosine. In some embodiments, the adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid.

In some embodiments, an adenosine deaminase may be a variant of a naturally-occurring adenine deaminase. Thus, in some embodiments, an adenosine deaminase useful with the invention may be about 70% to 100% identical to a wild type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in an organism.

In some embodiments, an adenine deaminase domain may be a wild type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a wild type E. coli TadA comprises the amino acid sequence of SEQ ID NO:18. In some embodiments, a mutated/evolved E. coli TadA* comprises the amino acid sequence of SEQ ID NOs:19-22. In some embodiments, a polynucleotide encoding a TadA/TadA* may be codon optimized for expression in an organism.

A cytosine deaminase (or cytidine deaminase) useful with this invention may be any known or later identified cytosine deaminase from any organism (see, e.g., U.S. Pat. No. 10,167,457, which is incorporated by reference herein for its disclosure of cytosine deaminases). In some embodiments, the at least one polypeptide or protein domain may be a cytosine deaminase polypeptide or domain. In some embodiments, a cytosine deaminase polypeptide/domain may be an apolipoprotein B mRNA editing catalytic polypeptide-like (APOBEC) domain. In some embodiments, a polypeptide of interest may comprise at least one polypeptide or protein domain having glycosylase inhibitor activity. In some embodiments, the polypeptide of interest may be a uracil-DNA glycosylase inhibitor (UGI) polypeptide/domain. In some embodiments, a nucleic acid construct encoding a modified LbCas12a nuclease of this invention and a cytosine deaminase domain (e.g., encoding a fusion protein comprising a modified LbCas12a nuclease and a cytosine deaminase domain) may further encode a uracil-DNA glycosylase inhibitor (UGI), wherein the UGI is codon optimized for expression in an organism. In some embodiments, the invention provides a fusion protein comprising a modified LbCas12a nuclease, a cytosine deaminase domain, and a UGI and/or one or more polynucleotides encoding the same, optionally wherein the one or more polynucleotides may be codon optimized for expression in an organism.

A cytosine deaminase catalyzes the hydrolytic deamination of cytidine or deoxycytidine to uridine or deoxyuridine, respectively. In some embodiments, a deaminase or deaminase domain may be a cytidine deaminase domain, catalyzing the hydrolytic deamination of cytosine to uracil. In some embodiments, a cytosine deaminase may be a variant of a naturally-occurring cytosine deaminase, including but not limited to a primate (e.g., a human, monkey, chimpanzee, gorilla), a dog, a cow, a rat or a mouse. Thus, in some embodiments, an cytosine deaminase useful with the invention may be about 70% to about 100% identical to a wild type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase). In some embodiments, a polynucleotide encoding a cytosine deaminase polypeptide/domain may be codon optimized for expression in an organism.

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDA1 (e.g., At2g19570), and evolved versions of the same. In some embodiments, the cytosine deaminase may be an APOBEC1 deaminase having the amino acid sequence of SEQ ID NO:23, SEQ ID NO:44 or SEQ ID NO:46. In some embodiments, the cytosine deaminase may be an APOBEC3A deaminase having the amino acid sequence of SEQ ID

NO:24. In some embodiments, the cytosine deaminase may be an CDA1 deaminase, optionally a CDA1 having the amino acid sequence of SEQ ID NO:25 or SEQ ID NO:43. In some embodiments, the cytosine deaminase may be a FERNY deaminase, optionally a FERNY having the amino acid sequence of SEQ ID NO:42 or SEQ ID NO:45. In some embodiments, the cytosine deaminase may be a human activation induced deaminase (hAID) having the amino acid sequence of SEQ ID NO:47 or SEQ ID NO:48. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 30 98%, 99%, 99.5% or 100% identical) to the amino acid sequence of a naturally occurring cytosine deaminase (e.g., an evolved deaminase). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of SEQ ID NO:23, SEQ ID NO:24, SEQ ID NO:25 or SEQ ID NOs:42-48 (e.g., at least 80%, at least 85%, at least 90%, at least 92%, at least 95%, at least 96%, at least 97%, at least 98%, at least 99%, or at least 99.5% identical to the amino acid sequence of SEQ ID NO:23, SEQ ID NO:24, SEQ ID NO:25 or SEQ ID NOs:42-48). In some embodiments, a polynucleotide encoding a cytosine deaminase may be codon optimized for expression in an organism and the codon optimized polypeptide may be about 70% to 99.5% identical to the reference polynucleotide.

A “uracil glycosylase inhibitor” (UGI) useful with the invention may be any protein that is capable of inhibiting a uracil-DNA glycosylase base-excision repair enzyme. In some embodiments, a UGI domain comprises a wild type UGI or a fragment thereof. In some embodiments, a UGI domain useful with the invention may be about 70% to about 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI domain. In some embodiments, a UGI domain may comprise the amino acid sequence of SEQ ID NO:26 or a polypeptide having about 70% to about 99.5% identity to the amino acid sequence of SEQ ID NO:26 (e.g., at least 80%, at least 85%, at least 90%, at least 92%, at least 95%, at least 96%, at least 97%, at least 98%, at least 99%, or at least 99.5% identical to the amino acid sequence of SEQ ID NO:26). For example, in some embodiments, a UGI domain may comprise a fragment of the amino acid sequence of SEQ ID NO:26 that is 100% identical to a portion of consecutive nucleotides (e.g., about 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO:26. In some embodiments, a UGI domain may be a variant of a known UGI (e.g., SEQ ID NO:26) having 70% to about 99.5% identity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% identity, and any range or value therein) to the known UGI. In some embodiments, a polynucleotide encoding a UGA may be codon optimized for expression in an organism and the codon optimized polypeptide may be about 70% to about 99.5% identical to the reference polynucleotide.

In some embodiments, a modified LbCas12a nuclease may comprise a mutation in its nuclease active site (e.g., RuvC). A modified LbCas12a nuclease having a mutation in its nuclease active site(s) and no longer comprising nuclease activity are commonly referred to as “dead,” e.g., dLbCas12a. In some embodiments, a modified LbCas12a domain or polypeptide having a mutation in its nuclease active site(s) may have impaired activity or reduced activity (e.g., nickase activity) as compared to the same LbCas12a nuclease without the mutation.

The modified LbCas12a nucleases of the invention may be used in combination with a guide RNA (gRNA, CRISPR array, CRISPR RNA, crRNA), designed to function with the modified LbCas12a nuclease, to modify a target nucleic acid. A guide nucleic acid useful with this invention comprises at least a spacer sequence and a repeat sequence. The guide nucleic acid is capable of forming a complex with the LbCas12a nuclease domain encoded and expressed by a polynucleotide/nucleic acid construct of the invention encoding a modified LbCas12a nuclease and the spacer sequence is capable of hybridizing to a target nucleic acid, thereby guiding the nucleic acid construct (e.g., the modified LbCas12a nuclease (and/or a polypeptide of interest)) to the target nucleic acid, wherein the target nucleic acid may be modified (e.g., cleaved or edited) or modulated (e.g., modulating transcription) by the modified LbCas12a nuclease (and/or an encoded deaminase domain and/or polypeptide of interest). As an example, a nucleic acid construct encoding an LbCas12a domain linked to a cytosine deaminase domain (e.g., a fusion protein) may be used in combination with an LbCas12a guide nucleic acid to modify a target nucleic acid, wherein the cytosine deaminase domain of the fusion protein deaminates a cytosine base in the target nucleic acid, thereby editing the target nucleic acid. In a further example, a nucleic acid construct encoding an LbCas12a domain linked to an adenine deaminase domain (e.g., a fusion protein) may be used in combination with an LbCas12a guide nucleic acid to modify a target nucleic acid, wherein the adenine deaminase domain of the fusion protein deaminates an adenosine base in the target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target nucleic acid (e.g., protospacer), and at least one repeat sequence (e.g., a repeat of a Type V Cas12a CRISPR-Cas system, or a fragment or portion thereof), wherein the repeat sequence may be linked to the 5′ end and/or the 3′ end of the spacer sequence. The design of a gRNA of this invention may be based on a Type V Cas12a system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one “repeat sequence-spacer” sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type Cas12a locus (e.g., a LbCas12a locu.) or a repeat sequence of a synthetic crRNA that is functional with the LbCas12a nuclease encoded by the nucleic acid constructs of the invention. A repeat sequence useful with this invention can be any known or later identified repeat sequence of a Cas12a locus or it can be a synthetic repeat designed to function in a Cas12a Type V CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from a wild-type V CRISPR-Cas locus (e.g., a wild type Cas12a locus). A repeat sequence from a wild-type Cas12a locus may be determined through established algorithms, such as using the CRISPRfinder offered through CRISPRdb (see, Grissa et al. Nucleic Acids Res. 35(Web Server issue):W52-7). In some embodiments, a repeat sequence or portion thereof is linked at its 3′ end to the 5′ end of a spacer sequence, thereby forming a repeat-spacer sequence (e.g., guide RNA, crRNA).

In some embodiments, a repeat sequence comprises, consists essentially of, or consists of at least 10 nucleotides depending on the particular repeat and whether the guide RNA comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein; e.g., about). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100, or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more consecutive nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% identity (e.g., at least about 90%, 91%, 92%, 93%, 94%, 95%, 96%, 97%, 98%, 99%, or more) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g, protospacer). The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, and any range or value therein)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have about 70% complementarity to a target nucleic acid. In other embodiments, the spacer nucleotide sequence can have about 80% complementarity to a target nucleic acid. In still other embodiments, the spacer nucleotide sequence can have about 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., about 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that may be at least about 15 nucleotides to about 30 nucleotides in length. In some embodiments, the spacer may be about 20, 21, 22, 23, 24, or 25 nucleotides in length. In some embodiments, the spacer may be 23 nucleotides in length.

In some embodiments, the 5′ region of a spacer sequence of a guide RNA may be identical to a target nucleic acid, while the 3′ region of the spacer may be substantially complementary to the target nucleic acid (e.g., Type V CRISPR-Cas), or the 3′ region of a spacer sequence of a guide RNA may be identical to a target nucleic acid, while the 5′ region of the spacer may be substantially complementary to the target nucleic acid (e.g., Type II CRISPR-Cas), and therefore, the overall complementarity of the spacer sequence to the target nucleic acid may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence, may be 100% complementary to the target nucleic acid, while the remaining nucleotides in the 3′ region of the spacer sequence may be substantially complementary (e.g., at least about 70% complementary) to the target nucleic acid. In some embodiments, the first 1 to 8 nucleotides (e.g., the first 1, 2, 3, 4, 5, 6, 7, 8, nucleotides, and any range therein) of a 5′ end of the spacer sequence may be 100% complementary to the target nucleic acid, while the remaining nucleotides in the 3′ region of the spacer sequence may be substantially complementary (e.g., at least about 50% complementary (e.g., about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleic acid.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refer to a region of an organism's genome that is fully complementary (100% complementary) or substantially complementary (e.g., at least 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more, and any range or value therein)) to a spacer sequence in a guide RNA of this invention. In some embodiments, a target region useful for a Type V CRISPR-Cas system (e.g., LbCas12a) is located immediately 3′ to a PAM sequence in the genome of the organism (e.g., a plant genome, an animal genome, a bacterial genome). In some embodiments, a target region may be selected from any at least 15 consecutive nucleotides (e.g., 15, 16, 17, 18, 19, 20, 21, 22, 23, 24, 25, 26, 27, 28, 29, 30 or more nucleotides, and any range or value therein; e.g., about 19 to about 25 nucleotides, about 20 to about 24 nucleotides in length, and the like) located immediately adjacent to a PAM sequence.

A “protospacer sequence” refers to the target nucleic acid and specifically to the portion of the target nucleic acid (e.g., or target region in the genome) that is fully or substantially complementary (and hybridizes) to the spacer sequence of the CRISPR repeat-spacer sequences (e.g., guide RNAs, CRISPR arrays, crRNAs).

In the case of Type V CRISPR-Cas Cas12a systems, the protospacer sequence is flanked (immediately adjacent to) a protospacer adjacent motif (PAM). The PAM is located at the 5′ end on the non-target strand and at the 3′ end of the target strand (see below, as an example).

```
(SEQ ID NO: 27)
      5′-NNNNNNNNNNNNNNNNNNN-3′ RNA Spacer
              ||||||||||||||||||||
(SEQ ID NO: 28)
3′AAANNNNNNNNNNNNNNNNNNN-5′ Target strand
    ||||
(SEQ ID NO: 29)
5′TTTNNNNNNNNNNNNNNNNNNN-3′ Non-target strand
```

Canonical Cas12a PAMs are T rich. In some embodiments, a canonical Cas12a PAM sequence may be 5′-TTN, 5′-TTTN, or 5′-TTTV.

The polypeptides, fusion proteins and/or systems of the invention may be encoded by polynucleotides or nucleic acid constructs. In some embodiments, a polynucleotide/nucleic acid construct encoding the polypeptides, fusion proteins and/or systems of the invention may be operably associated with regulatory elements (e.g., promotors, terminators and the like) for expression in an organism of interest and/or a cell of an organism of interest as described herein. In some embodiments, a polynucleotide/nucleic acid construct encoding a polypeptide, fusion protein and/or system of the invention may be codon optimized for expression in an organism.

In some embodiments, the present invention provides a complex comprising (a) a modified LbCas12a polypeptide of the invention or a fusion protein of the invention and (b) a guide nucleic acid (e.g., CRISPR RNA, CRISPR DNA, crRNA, crDNA).

In some embodiments, the present invention provides a composition comprising (a) a modified LbCas12a polypeptide of the invention or a fusion protein of the invention and (b) a guide nucleic acid.

In some embodiments, the present invention provides expression cassettes and/or vectors comprising the polynucleotides/nucleic acid constructs of the invention. In some embodiments, expression cassettes and/or vectors comprising the polynucleotides/nucleic acid constructs of the invention and/or one or more guide nucleic acids may be provided. In some embodiments, a nucleic acid construct encoding a modified CRISPR-Cas nuclease and/or a fusion protein comprising a modified CRISPR-Cas nuclease of the invention may be comprised in the same or a separate expression cassette or vector from that comprising the guide nucleic acid. When the nucleic acid construct is comprised in a separate expression cassette or vector from that comprising the guide nucleic acid, a target nucleic acid may be contacted with (e.g., provided with) the expression cassette or vector comprising the nucleic acid construct of the invention prior to, concurrently with, or after the expression cassette comprising the guide nucleic acid is provided (e.g., contacted with the target nucleic acid).

In some embodiments, the present invention provides expression cassettes and/or vectors encoding compositions and/or complexes of the invention or comprising systems of the invention.

In some embodiments, the polynucleotides, nucleic acid constructs, expression cassettes and/or vectors of the invention that are optimized for expression in an organism may be about 70% to about 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%, and any value or range therein) to the polynucleotides, nucleic acid constructs, expression cassettes and/or vectors encoding the same modified CRISPR-Cas nuclease or fusion protein of the invention but which has not been codon optimized for expression in an organism. An organism for which a polynucleotide or nucleic acid construct may be optimized may include but is not limited to, an animal, a plant, a fungus, an archaeon, or a bacterium. In some embodiments, a polynucleotide or nucleic acid construct of the invention is codon optimized for expression in a plant.

In some embodiments, the invention provides cells comprising one or more polynucleotides, guide nucleic acids, nucleic acid constructs, systems, expression cassettes and/or vectors of the invention.

The nucleic acid constructs of the invention (e.g., encoding a modified CRISPR-Cas nuclease of the invention and/or a fusion protein comprising a modified CRISPR-Cas nuclease of the invention) and expression cassettes/vectors comprising the same may be used for modifying target nucleic acids and/or their expression in vivo (e.g., in an organism or the cell of an organism; e.g., a plant) and in vitro (e.g., in a cell or a cell free system).

The present invention further provides methods for altering the PAM specificity of a Cas12a polypeptide. In some embodiments, a method for altering PAM specificity is provided that comprises introducing a mutation into the Cas12a polypeptide wherein the mutation is at amino acid residue K116, K120, K121, D122, E125, T148, T149, T152, D156, E159, Q529, D535, K538, D541, Y542, L585, K591, M592, K595, V596, 5599, K600, K601, Y616, Y646, W649 with reference to the position numbering of SEQ ID NO:1. In some embodiments, the mutation that is introduced into the Cas12a polypeptide is K116R, K116N, K120R, K120H, K120N, K120T, K120Y, K120Q, K121S, K121T, K121H, K121R, K121G, K121D, K121Q, D122R, D122K, D122H, D122E, D122N, E125R, E125K, E125Q, E125Y, T148H, T148S, T148A, T148C, T149A, T149C, T149S, T149G, T149H, T149P, T149F, T149N, T149D, T149V, T152R, T152K, T152W, T152Y, T152H, T152Q, T152E, T152L, T152F, D156R, D156K, D156Y, D156W, D156Q, D156H, D156I, D156V, D156L, D156E, E159K, E159R, E159H, E159Y, E159Q, Q529N, Q529T, Q529H, Q529A, Q529F, Q529G, Q529G, Q529S, Q529P, Q529W, Q529D, G532D, G532N, G532S, G532H, G532F, G532K, G532R, G532Q, G532A, G532L, G532C, D535N, D535H, D535V, D535T, D535,S D535A, D535W, D535K, K538R K538V, K538Q, K538W, K538Y, K538F, K538H, K538L, K538M, K538C, K538G, K538A, K538P, D541N, D541H, D541R, D541K, D541Y, D541I, D541A, D541S, D541E, Y542R, Y542K, Y542H, Y542Q, Y542F, Y542L, Y542M, Y542P, Y542V, Y542N, Y542T, L585G, L585H, L585F, K591W, K591F, K591Y, K591H, K591R, K591S, K591A, K591G, K591P, M592R, M592K, M592Q, M592E, M592A, K595R, K595Q, K595Y, K595L, K595W, K595H, K595E, K595S, K595D, K595M, V596T, V596H, V596G, V596A, S599G, S599H, S599N, S599D, K600R, K600H, K600G, K601R, K601H, K601Q, K601T, Y616K, Y616R, Y616E, Y616F, Y616H, Y646R, Y646E, Y646K, Y646H, Y646Q, Y646W, Y646N, W649H, W649K, W649Y, W649R, W649E, W649S, W649V, and/or W649T with reference to the position numbering of SEQ ID NO:1. In some embodiments, the mutation that is introduced into the Cas12a polypeptide is at amino acid residue position K116, K120, K121, D122, E125, T152, D156, E159, G532, D535, K538, D541, and/or K595 with reference to position numbering of SEQ ID NO:1, optionally wherein the mutation is K116R, K116N, K120Y, K121S, K121R, D122H, D122N, E125K, T152R, T152K, T152Y, T152Q, T152E, T152F, D156R, D156W, D156Q, D156H, D156I, D156V, D156L, D156E, E159K, E159R, G532N, G532S, G532H, G532K, G532R, G532L, D535N, D535H, D535T, D535,S D535A, D535W, K538R K538V, K538Q, K538W, K538Y, K538F, K538H, K538L, K538M, K538C, K538G, K538A, D541E, K595R, K595Q, K595Y, K595W, K595H, K595S, and/or K595M with reference to position numbering of SEQ ID NO:1. The mutation that is introduced may be a single mutation or it may be a combination of two or more mutations. As would be understood, any single Cas12a polypeptide having two or more mutations would comprise only a single mutation at any given position. In some embodiments, the Cas12a polypeptide that is altered in PAM specificity by the methods of this invention is a LbCas12a polypeptide (Lachnospiraceae bacterium).

Modified Cas12a polypeptides or nucleases (e.g., LbCas12a nucleases) of the invention may be used for modifying a target nucleic acid in a cell or a cell free system (e.g., altering the target nucleic acid, altering the genome of the cell/organism). Accordingly, in some embodiments, a method of modifying a target nucleic acid is provided, the method comprising: contacting the target nucleic acid with: (a)(i) a modified LbCas12a polypeptide of the invention, or a fusion protein of the invention (e.g., a modified LbCas12a polypeptide of the invention and a polypeptide of interest (e.g., a deaminase)), and (ii) a guide nucleic acid; (b) a complex of the invention comprising (i) a modified LbCas12a polypeptide or a fusion protein of the invention, and (ii) a guide nucleic acid; (c) a composition comprising (i) a modified LbCas12a polypeptide of the invention, or a fusion protein of the invention, and (ii) a guide nucleic acid; and/or (d) a system of the invention, thereby modifying the target nucleic acid. In some embodiments, a method of modifying/altering the genome of a cell or organism is provided, the method comprising: contacting a target nucleic acid in the genome of the cell/organism with: (a)(i) a modified LbCas12a polypeptide of the invention, or a fusion protein of the invention (e.g., a modified LbCas12a polypeptide of the invention and a polypeptide of interest (e.g., a deaminase)), and (ii) a guide nucleic acid; (b) a complex of the invention comprising (i) a modified LbCas12a polypeptide or a fusion protein of the invention, and (ii) a guide nucleic acid; (c) a composition comprising (i) a modified CRISPR-Cas nuclease (e.g., a modified LbCas12a polypeptide) of the invention, or a fusion protein of the invention, and (ii) a guide nucleic acid; and/or (d) a system of the invention, thereby modifying/altering the genome of a cell or organism. In some embodiments, the cell or organism is a plant cell or a plant.

In some embodiments, a method of modifying a target nucleic acid is provided, the method comprising: contacting a cell or a cell free system comprising the target nucleic acid with: (a)(i) a polynucleotide of the invention (e.g., encoding a modified LbCas12a polypeptide of the invention, or encoding fusion protein comprising a modified LbCas12a polypeptide of the invention and a polypeptide of interest (e.g., a deaminase)), or an expression cassette or vector comprising the same, and (ii) a guide nucleic acid, or an expression cassette and/or vector comprising the same; and/or (b) a nucleic acid construct encoding a complex of the invention comprising a modified LbCas12a polypeptide of the invention, or fusion protein comprising a modified LbCas12a polypeptide of the invention and a polypeptide of interest (e.g., a deaminase), or an expression cassette and/or vector comprising the same, wherein the contacting is carried out under conditions whereby the polynucleotide and/or nucleic acid construct is/are expressed and the modified LbCas12a polypeptide and/or fusion protein is/are produced, which forms a complex with the guide nucleic acid, thereby modifying the target nucleic acid. In some embodiments, a method of modifying/altering the genome of a cell and/or organism is provided, the method comprising: contacting a cell and/or cell in an organism comprising the target nucleic acid with: (a)(i) a polynucleotide of the invention (e.g., encoding a modified LbCas12a polypeptide of the invention, or encoding fusion protein comprising a modified LbCas12a polypeptide of the invention and a polypeptide of interest (e.g., a deaminase)), or an expression cassette or vector comprising the same, and (ii) a guide nucleic acid, or an expression cassette and/or vector comprising the same; and/or (b) a nucleic acid construct encoding a complex of the invention comprising a modified LbCas12a polypeptide of the invention, or fusion protein comprising a modified LbCas12a polypeptide of the invention and a polypeptide of interest (e.g., a deaminase), or an expression cassette and/or vector comprising the same, wherein the contacting is under conditions whereby the polynucleotide and/or nucleic acid construct is/are expressed and the modified LbCas12a polypeptide and/or fusion protein is/are produced, which form(s) a complex with the guide nucleic acid, thereby modifying the target nucleic acid.

In some embodiments, the present invention provides a method of editing a target nucleic acid, the method comprising: contacting the target nucleic acid with: (a)(i) a fusion protein of the present invention (comprising a modified LbCas12a polypeptide of the invention and a polypeptide of interest (e.g., a deaminase), and (a)(ii) a guide nucleic acid; (b) a complex comprising a fusion protein of the present invention, and a guide nucleic acid; (c) a composition comprising the fusion protein of the present invention and a guide nucleic acid; and/or (d) a system of the invention, thereby editing the target nucleic acid.

In some embodiments, the present invention provides a method of editing a target nucleic acid, the method comprising: contacting a cell or a cell free system comprising the target nucleic acid with: (a)(i) a polynucleotide encoding a fusion protein of the invention (e.g., a modified LbCas12a polypeptide of the invention and a polypeptide of interest (e.g. a deaminase)) or an expression cassette and/or vector comprising the same, and (a)(ii) a guide nucleic acid, or an expression cassette and/or vector comprising the same; (b) a nucleic acid construct encoding a complex comprising a fusion protein of the present invention, and a guide nucleic acid, or an expression cassette and/or vector comprising the same; and/or (c) a system of the present invention wherein the contacting is carried out under conditions whereby the polynucleotide and/or nucleic acid construct is/are expressed and the modified CRISPR-Cas nuclease and/or fusion protein is/are produced, which form(s) a complex with the guide nucleic acid, thereby editing the target nucleic acid.

The CRISPR-Cas nucleases having modified PAM recognition specificities may be utilized in many ways including, but not limited to, creating indels (NHEJ), in homology directed repair, as a genome recognition element without a nuclease function (dead Cpf1), as a genome recognition element with a partially functional nuclease (nickase Cpf1), in fusion proteins for catalytic editing of genomic DNA (DNA base editors), in fusions proteins for catalytic editing of RNA (RNA base editors), for targeting of other macromolecules to specific genomic regions; for targeting of small chemicals to specific genomic regions, for labeling of specific genomic regions and/or for CRISPR-directed genomic recombination strategies.

When provided on different nucleic acid constructs, expression vectors, and/or vectors, a nucleic acid construct of the invention may be contacted with a target nucleic acid prior to, concurrently with or after contacting the target nucleic acid with a guide nucleic acid.

The modified CRISPR-Cas nucleases of the invention and polypeptides and nucleic acid constructs encoding the same may be used for modifying a target nucleic acid in any organism, including but not limited to, an animal, a plant, a fungus, an archaeon, or a bacterium. An animal can include, but is not limited to, a mammal, an insect, a fish, a bird, and the like. Exemplary mammals for which this invention may be useful include, but are not limited to, primates (human and non-human (e.g., a chimpanzee, baboon, monkey, gorilla, etc.)), cats, dogs, mice, rats, ferrets, gerbils, hamsters, cows, pigs, horses, goats, donkeys, or sheep.

A target nucleic acid of any plant or plant part may be modified and/or edited (e.g., mutated, e.g., base edited, cleaved, nicked, and the like) using the nucleic acid constructs of the invention. Any plant (or groupings of plants, for example, into a genus or higher order classification) may be modified using the nucleic acid constructs of this invention including an angiosperm, a gymnosperm, a monocot, a dicot, a C3, C4, CAM plant, a bryophyte, a fern and/or fern ally, a microalgae, and/or a macroalgae. A plant and/or plant part useful with this invention may be a plant and/or plant part of any plant species/variety/cultivar. The term “plant part,” as used herein, includes but is not limited to, embryos, pollen, ovules, seeds, leaves, stems, shoots, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast. A plant cell can be in the form of an isolated single cell or can be a cultured cell or can be a part of a higher-organized unit such as, for example, a plant tissue or a plant organ.

Non-limiting examples of plants useful with the present invention include turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, Chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (e.g., sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (e.g., wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (e.g., field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil seed plant (e.g., rape, canola, mustard, poppy, olive, sunflower, coconut, castor oil plant, cocoa bean, groundnut, oil palm, soybean, Camelina, and the like), duckweed, Arabidopsis, a fiber plant (cotton, flax, hemp, jute), Cannabis (e.g., Cannabis sativa, Cannabis indica, and Cannabis ruderalis), lauraceae (e.g., cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, the nucleic acid constructs of the invention and/or expression cassettes and/or vectors encoding the same may be used to modify maize, soybean, wheat, canola, rice, tomato, pepper, sunflower, raspberry, blackberry, black raspberry and/or cherry.

The present invention further comprises a kit to carry out methods of this invention. A kit of this invention can comprise reagents, buffers, and/or apparatus for mixing, measuring, sorting, labeling, etc, as well as instructions and the like as would be appropriate for modifying a target nucleic acid.

In some embodiments, the invention provides a kit comprising one or more polynucleotides and/or nucleic acid constructs of the invention, and/or expression cassettes and/or vectors comprising the same, with optional instructions for the use thereof. In some embodiments, a kit may further comprise a polypeptide of interest and/or polynucleotide encoding the same and expression cassette and/or vector comprising the same. In some embodiments, a guide nucleic acid may be provided on the same expression cassette and/or vector as a nucleic acid construct of the invention. In some embodiments, a guide nucleic acid may be provided on a separate expression cassette or vector from that comprising the nucleic acid construct of the invention.

Accordingly, in some embodiments, kits are provided comprising a nucleic acid construct comprising (a) a polynucleotide encoding a modified CRISPR-Cas nuclease as provided herein and (b) a promoter that drives expression of the polynucleotide of (a). In some embodiments, the kit may further comprise a nucleic acid construct encoding a guide nucleic acid, wherein the construct comprises a cloning site for cloning into the backbone of the guide nucleic acid a nucleic acid sequence that is identical or complementary to a target nucleic acid sequence.

In some embodiments, the kit may comprise a nucleic acid construct comprising/encoding one or more nuclear localization signals, wherein the nuclear localization signals are fused to the CRISPR-Cas nuclease. In some embodiments, kits are provided comprising a nucleic acid construct of the invention encoding a modified CRISPR-Cas nuclease of the invention or, and/or an expression cassette and/or vector comprising the same, wherein the nucleic acid constructs, expression cassettes and/or vectors may further encode one or more selectable markers useful for identifying transformants (e.g., a nucleic acid encoding an antibiotic resistance gene, herbicide resistance gene, and the like). In some embodiments, the nucleic acid construct may be an mRNA that encodes one or more introns within the encoded CRISPR-Cas nuclease. In some embodiments, a kit may comprise promoters and promoters with introns for use in expression of the polypeptides and nucleic acid constructs of the invention.

### Methods For Modifying PAM Specificities of CRISPR-Cas Nucleases and Related Compositions

CRISPR-Cas systems are directed towards target nucleic acids using two major criteria: homology of the guide RNA to the targeted DNA sequence, and presence of a protospacer adjacent motif (PAM) of a particular sequence. Different CRISPR-Cas nucleases have different PAM sequence requirements, such as NGG for SpCas9 or TTTV (where V is any non-thymidine nucleotide) for LbCas12a (Cpf1). Screening new CRISPR nucleases or mutants thereof for their PAM requirements can be complicated and unpredictable because so many iterations are possible. In vitro assays, particularly PAM Determination Assays (PAMDA), may be used to screen PAM specificity for any particular CRISPR nuclease or mutant thereof. These assays rely on a randomized portion of DNA that is adjacent to a defined/known protospacer sequence. Guide RNAs can be designed to target the known protospacer sequence and if the randomized PAM region contains the appropriate DNA sequence (e.g., recognized by the CRISPR nuclease or mutants thereof), then the CRISPR nuclease can bind and cut the target.

The PAM recognition sites for CRISPR-Cas nucleases may be assessed using PAM site-depletion assays (e.g., PAM depletion assays) or PAM Determination Assays (PAMDA) (Kleinstiver et al. Nat Biotechnol 37: 276-282 (2019)). For PAM depletion assays, a library of plasmids bearing randomized nucleotides (base pairs) adjacent to a protospacer is tested for cleavage by a CRISPR nuclease in a bacterium (e.g., E. coli). The plasmids may comprise, for example, polynucleotides conferring antibiotic resistance that are adjacent to randomized PAM sequences. Those sequences which are not cut upon exposure to a CRISPR-Cas nuclease enable a cell to survive in the presence of an antibiotic due to the presence of the antibiotic resistance gene, whereas plasmids bearing targetable PAMs are cleaved and depleted from the library due to cell death. Sequencing of the surviving (uncleaved) population of plasmids enables the calculation of a post-selection PAM depletion value, which is compared to a library that has not been exposed to the CRISPR-Cas nuclease. Those sequences which are depleted from the pool of sequences in the experimental library contain the PAM sequence(s) recognized by the CRISPR-Cas nuclease.

Another method that may be used to identify PAM sequences is a PAM Determination Assay (PAMDA) (Kleinstiver et al. Nat Biotechnol 37: 276-282 (2019)). In this case, cleavage is performed outside of living cells. In PAMDA, a single DNA strand is synthesized with a randomized portion of nucleotides next to a defined protospacer sequence. An oligonucleotide is annealed to the 3′ end of the synthesized DNA strand and extended using an exonuclease minus (-exo) Klenow fragment, polymerizing over the defined and random sequences. This generates a duplex library that is then cut with restriction endonucleases and cloned into bacteria in order to amplify the total DNA. The plasmids are extracted and linearized with another restriction endonuclease to make a linear template. The template is contacted with a CRISPR-Cas nuclease-guide RNA complex. Only those sequences containing a PAM that is recognized by the CRISPR-Cas nuclease will be cleaved. Both the experimental library and the control library (not exposed to the CRISPR-Cas nuclease) are then amplified via PCR. Only the sequences that are not cleaved by the CRISPR-Cas nuclease will be amplified. The PCR amplified sequences from the control library and the experimental library (treated with CRISPR-Cas nuclease) are sequenced and compared. The PAM sequences that are present in the control (not exposed to CRISPR-Cas nuclease) library but not in the experimental library are the PAM sequences that are recognized by the CRISPR-Cas nuclease (thereby, allowing the protospacer to be cleaved).

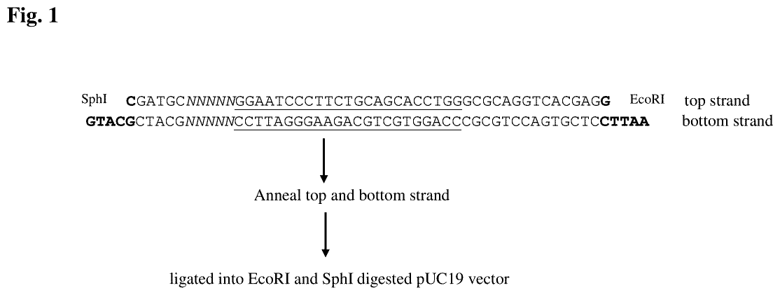

For the assessment of nuclease requirements in vitro, a randomized PAM library is prepared. Steps described for this method involve preparation of an unbiased randomized DNA library containing all PAM sequences to be evaluated, cloning into a plasmid, introduction of the library into bacteria to increase the total amount of starting DNA, extraction of the plasmids, linearization of the plasmids with a restriction enzyme to remove supercoiling, exposing the linearized molecules to the CRIPSR-Cas nuclease, amplifying the fragments (e.g., PCR), and finally sequencing analysis (e.g., next generation sequencing, NGS). The initial steps of generating an unbiased library and restriction digests require at least two restriction enzymes, Klenow extension, and cleaning of the products before ligating into a vector. Using two to three restriction enzymes typically eliminates some PAM sequences from the library, which introduces bias into the library. In addition, subsequent Klenow extension and cleanup steps can also eliminate PAM sequences, thereby introducing further bias into the library. In order to avoid the loss of PAM sequences and to generate a more complete and unbiased library, the present invention provides a new method for generating randomized PAM libraries using overlapping solid-state synthesized oligonucleotides (e.g., annealed oligonucleotides) with overhangs (see, e.g., FIG. 1b) instead of restriction endonucleases and Klenow extension. The randomized PAM libraries produced using the methods of the invention can then be used to test the PAM specificities of CRISPR-Cas nucleases with more accuracy than was previously available with libraries produced via prior art methods.

Accordingly, in some embodiments, the present invention provides a method of constructing a randomized DNA library comprising double stranded nucleic acid molecules for determining protospacer adjacent motif (PAM) requirements/specificity of a CRISPR-Cas nuclease having a PAM recognition site at the 5′ end of the protospacer, the method comprising: preparing two or more double stranded nucleic acid molecules comprising the following steps: (a) synthesizing a non-target oligonucleotide (first) strand and a target oligonucleotide (second) strand for each of the two or more double stranded nucleic acid molecules, wherein the non-target oligonucleotide strand comprises, 5′ to 3′: (i) a first sequence having about 5 to about 15 nucleotides (e.g., about 5, 6, 7, 8, 9, 10, 11, 12, 13, 14, or 15 nucleotides, and any range therein), (ii) (ii) a second sequence having at least four randomized nucleotides (e.g., at least 4, 5, 6, 7, 8, 9, 10, or more, and any range therein), (ii) a protospacer sequence comprising about 16 to about 25 nucleotides (e.g., about 16, 17, 18, 19, 20, 21, 22, 23, 24, or 25 nucleotides, and any range therein), and (iv) a third sequence having about 5 to about 20 nucleotides (e.g., about 5, 6, 7, 8, 9, 10, 11, 12, 13, 14, 15, 16, 17, 18, 19, 20 nucleotides, and any range therein), wherein the first sequence having about 5 to 15 nucleotides of (i) is immediately adjacent to the 5′ end of the second sequence of (ii), the second sequence of (ii) is immediately adjacent to the 5′ end of the protospacer sequence of (iii), and the protospacer sequence is immediately adjacent to the 5′ end of the third sequence of (iv); and the target oligonucleotide (second) strand is complementary to the non-target oligonucleotide strand; and (b) annealing the non-target oligonucleotide strand to the complementary target oligonucleotide strand to produce a double stranded nucleic acid molecule, wherein the first sequence comprises a restriction site (at its 5′ end) and the third sequence comprises a restriction site (at its 3′ end), wherein the first sequence (i), the protospacer sequence (iii) and the third sequence (iv) of each of the two or more double stranded nucleic acid molecules is identical, thereby constructing the randomized DNA library comprising double stranded nucleic acid molecules. In some embodiments, the target strand and/or the non-target strand may be 5′ phosphorylated.

In some embodiments, the present invention provides a method of constructing a randomized DNA library comprising double stranded nucleic acid molecules for determining protospacer adjacent motif (PAM) requirements/specificity of a CRISPR-Cas nuclease having a PAM recognition site at the 3′ end of the protospacer, the method comprising: preparing two or more double stranded nucleic acid molecules comprising the following steps: (a) synthesizing a non-target oligonucleotide (first) strand and a target oligonucleotide (second) strand for each of the two or more double stranded nucleic acid molecules, wherein the non-target oligonucleotide strand comprises, 5′ to 3′: (i) a first sequence having about 5 to about 20 nucleotides (e.g., about 5, 6, 7, 8, 9, 10, 11, 12, 13, 14, 15, 16, 17, 18, 19, 20 nucleotides, and any range therein), (ii) a protospacer sequence comprising about 16 to about 25 nucleotides (e.g., about 16, 17, 18, 19, 20, 21, 22, 23, 24, or 25 nucleotides, and any range therein), (iii) a second sequence having at least four randomized nucleotides (e.g., at least 4, 5, 6, 7, 8, 9, 10, or more, and any range therein), and (iv) a third sequence having about 5 to about 15 nucleotides (e.g., about 5, 6, 7, 8, 9, 10, 11, 12, 13, 14, 15 nucleotides, and any range therein), wherein the first sequence having about 5 to 20 nucleotides of (i) is immediately adjacent to the 5′ end of the protospacer sequence of (ii), the second sequence of (iii) is immediately adjacent to the 3′ end of the protospacer sequence of (iii), and the third sequence of (iv) is immediately adjacent to the 3′ end of the second sequence of (iii); and the target oligonucleotide (second) strand is complementary to the non-target oligonucleotide strand; and (b) annealing the non-target oligonucleotide strand to the complementary target oligonucleotide strand to produce a double stranded nucleic acid molecule, wherein the first sequence (i) comprises a restriction site (at its 5′ end) and the third sequence (iv) comprises a restriction site (at its 3′ end), wherein the first sequence (i), the protospacer sequence (ii) and the third sequence (iv) of each of the two or more double stranded nucleic acid molecules is identical, thereby constructing the randomized DNA library comprising double stranded nucleic acid molecules. In some embodiments, the target strand and/or the non-target strand may be 5′ phosphorylated.

In some embodiments, the double stranded nucleic acid molecules may be ligated into a vector to produce a vector comprising the randomized DNA library. In some embodiments, the vector may be a high copy number vector. In some embodiments, the randomized DNA library may be amplified by, for example, introducing the vector comprising the randomized DNA library into one or more bacterial cells and culturing the one or more bacterial cells. In some embodiments, the vector comprising the randomized DNA library may be isolated from the one or more bacterial cells after culturing. The isolated vector may then be linearized (e.g., by contacting the vector with one or more restriction enzymes; e.g., ScaI or PfoI) for use in, for example, the analysis of the PAM recognition specificity of a CRISPR-Cas nuclease. In some embodiments, Pfo1 may be used to linearize the isolated vector.

In some embodiments, a randomized DNA library may be provided for determining protospacer adjacent motif (PAM) requirements/specificity of a CRISPR-Cas nuclease having a PAM recognition site on the 5′ end of protospacer, the randomized DNA library comprising two or more double stranded nucleic acid molecules each of which comprises: (a) a non-target oligonucleotide (first) strand and a target oligonucleotide (second) strand, wherein the non-target oligonucleotide strand comprises, 5′ to 3′: (i) a first sequence having about 5 to about 15 nucleotides (e.g., about 5, 6, 7, 8, 9, 10, 11, 12, 13, 14, or 15 nucleotides, and any range therein), (ii) a second sequence having at least four randomized nucleotides (e.g., at least 4, 5, 6, 7, 8, 9, 10, or more, and any range therein), (iii) a protospacer sequence comprising about 16 to about 25 nucleotides (.g., about 16, 17, 18, 19, 20, 21, 22, 23, 24, or 25 nucleotides, and any range therein), and (iv) a third sequence having about 5 to about 20 nucleotides (e.g., about 5, 6, 7, 8, 9, 10, 11, 12, 13, 14, 15, 16, 17, 18, 19, 20 nucleotides, and any range therein), wherein the first sequence having about 5 to 15 nucleotides of (i) is immediately adjacent to the 5′ end of the second sequence of (ii), the second sequence of (ii) is immediately adjacent to the 5′ end of the protospacer sequence of (iii), and the protospacer sequence is immediately adjacent to the 5′ end of the third sequence of (iv); and the target oligonucleotide (second) strand is complementary to the non-target oligonucleotide strand; and (b) the non-target oligonucleotide strand is annealed to the complementary target oligonucleotide strand to produce a double stranded nucleic acid molecule, wherein the first sequence comprises a restriction site (at its 5′ end) and the third sequence comprises a restriction site (at its 3′ end), wherein the first sequence (i), the protospacer sequence (iii) and the third sequence (iv) of each of the two or more double stranded nucleic acid molecules are identical. In some embodiments, the target strand and/or the non-target strand may be 5′ phosphorylated.

In some embodiments, a randomized DNA library may be provided for determining protospacer adjacent motif (PAM) requirements/specificity of a CRISPR-Cas nuclease having a PAM recognition site on the 3′ end of protospacer, the randomized DNA library comprising two or more double stranded nucleic acid molecules each of which comprises: (a) a non-target oligonucleotide (first) strand and a target oligonucleotide (second) strand, wherein the non-target oligonucleotide strand comprises, 5′ to 3′: (i) a first sequence having about 5 to about 20 nucleotides (e.g., about 5, 6, 7, 8, 9, 10, 11, 12, 13, 14, 15, 16, 17, 18, 19, 20 nucleotides, and any range therein), (ii) a protospacer sequence comprising about 16 to about 25 nucleotides (e.g., about 16, 17, 18, 19, 20, 21, 22, 23, 24, or 25 nucleotides, and any range therein), (iii) a second sequence having at least four randomized nucleotides (e.g., at least 4, 5, 6, 7, 8, 9, 10, or more, and any range therein), and (iv) a third sequence having about 5 to about 15 nucleotides (e.g., about 5, 6, 7, 8, 9, 10, 11, 12, 13, 14, 15 nucleotides, and any range therein), wherein the first sequence having about 5 to 20 nucleotides of (i) is immediately adjacent to the 5′ end of the protospacer sequence of (ii), the second sequence of (iii) is immediately adjacent to the 3′ end of the protospacer sequence of (iii), and the third sequence of (iv) is immediately adjacent to the 3′ end of the second sequence of (iii); and the target oligonucleotide (second) strand is complementary to the non-target oligonucleotide strand; and (b) the non-target oligonucleotide strand is annealed to the complementary target oligonucleotide strand to produce a double stranded nucleic acid molecule, wherein the first sequence comprises a restriction site (at its 5′ end) and the third sequence comprises a restriction site (at its 3′ end), wherein the first sequence (i), the protospacer sequence (ii) and the third sequence (iv) of each of the two or more double stranded nucleic acid molecules are identical. In some embodiments, the target strand and/or the non-target strand may be 5′ phosphorylated.

In some embodiments, the present invention provides a method of determining protospacer adjacent motif (PAM) specificity of a CRISPR-Cas nuclease, the method comprising: contacting the CRISPR-Cas nuclease with a randomized DNA library of the present invention; and sequencing the double stranded nucleic acid molecules of the randomized DNA library before (e.g., the control) and after contact with the CRISPR-Cas nuclease, wherein double stranded nucleic acid molecules present in the randomized DNA library prior to contact with the CRISPR-Cas nuclease but absent in the randomized DNA library after contact with the CRISPR-Cas nuclease identifies the PAM recognition sequence of the CRISPR-Cas nuclease, thereby determining the PAM specificity of the CRISPR-Cas nuclease.

In some embodiments, a method of determining protospacer adjacent motif (PAM) specificity of a CRISPR-Cas nuclease, comprises: contacting the CRISPR-Cas nuclease with a randomized DNA library of the present invention; sequencing the double stranded nucleic acid molecules of the randomized DNA library before (e.g., the control) and after contact with the CRISPR-Cas nuclease, and identifying the PAM recognition sequence of the nuclease, wherein identifying comprises comparing the double stranded nucleic acid molecules present in the library before contact with the CRISPR-Cas nuclease to the double stranded nucleic acid molecules present in the library after contact with the CRISPR-Cas nuclease and wherein the double stranded nucleic acid molecules present in the randomized DNA library before contact with the CRISPR-Cas nuclease but absent from the randomized DNA library after contact with the CRISPR-Cas nuclease identifies the PAM specificity of the CRISPR-Cas nuclease.

The results of sequencing of the randomized library prior to contact can serve as a control to the results of sequencing after contact. In some embodiments, determining the PAM specificity of a CRISPR-Cas nuclease may comprise performing nucleic acid sequencing. In some embodiments, the sequence may comprise next generation sequencing (NGS).

Any CRISPR-Cas nuclease may be used with the methods of this invention for modifying PAM recognition specificity. Accordingly, a CRISPR-Cas nuclease that may be modified to have a different PAM specificity as compared to wild type can include, but is not limited, to a Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 polypeptide or domain.

Cas12a is a Type V Clustered Regularly Interspaced Short Palindromic Repeats (CRISPR)-Cas nuclease originally identified in Prevotella spp. and Francisella spp. Cas12a (previously referred to as Cpf1) differs in several respects from the more well-known Type II CRISPR Cas9 nuclease. For example, Cas9 recognizes a G-rich protospacer adjacent motif (PAM) that is 3′ to its guide RNA (gRNA, sgRNA) binding site (protospacer, target nucleic acid, target DNA) (3′-NGG), while Cas12a recognizes a T-rich PAM that is located 5′ to the binding site (protospacer, target nucleic acid, target DNA) (5′-TTN, 5′-TTTN). In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a polypeptide or CRISPR Cas12a domain useful with this invention may be any known or later identified Cas12a nuclease (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12a polypeptide, or a fragment thereof, which comprises the guide nucleic acid binding domain of Cas12a and/or an active, inactive, or partially active DNA cleavage domain of Cas12a. In some embodiments, a Cas12a useful with the invention may comprise a mutation in the nuclease active site (e.g., RuvC site of the Cas12a domain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas12a polypeptide having a mutation in its nuclease active site may have impaired/reduced activity as compared to the same Cas12a polypeptide without the same mutation (e.g., nickase activity).

In some embodiments, a Cas12a domain can include, but is not limited to, the amino acid sequence of any one of SEQ ID NOs:1-17 (e.g., SEQ ID NOs:1, 2, 3, 4, 5, 6, 7, 8, 9, 10, 11, 12, 13, 14, 15, 16 and/or 17) or a polynucleotide encoding the same. In some embodiments, a fusion protein of the invention may comprise a Cas12a domain from Lachnospiraceae bacterium ND2006 Cas12a (LbCas12a) (e.g., SEQ ID NO:1).

A CRISPR Cas9 polypeptide or CRISPR Cas9 domain useful with this invention may be any known or later identified Cas9 nuclease. In some embodiments, a Cas9 polypeptide useful with this invention comprises at least 70% identity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to an amino acid sequence of any known Cas9. CRISPR-Cas9 systems are well known in the art and include, but are not limited to, Cas9 polypeptides from Legionella pneumophila str. Paris, Streptococcus thermophilus CNRZ1066, Streptococcus pyogenes MI, or Neisseria lactamica 020-06, and the like.

Other nucleases that may be useful with this invention for identifying novel PAM recognition sequence include but are not limited to, C2c1, C2c3, Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmrl, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

### Randomized Library

An example, is provided of the method of this invention for the efficient and cost-effective generation of libraries for in vitro cleavage assays (PAM Determination Assays (PAMDA)). Two libraries were generated for protospacers 1 and 2 (see Table 1). Oligonucleotides with a randomized 5 nucleotide sequence at the 5′-end were synthesized and verified (Integrated DNA Technologies) so that each protospacer sequence occupies an equal molar ratio (Table 1). The oligonucleotides for protospacer 1 (PM0518, PM0519) and for protospacer 2 (PM0520, PM0521) were annealed by placing the mixture in a thermal cycler at 95° C. for 5 mins with a cool down for 0.1° C./sec to 25° C./room temperature.

TABLE 1

| Protospacer | | |
|---|---|---|
| name | Oligo number | Sequence |
| PAMDA | Protospacer 1 | GGAATCCCTTCTGCAGCACCTGG |
| library 1 | | (SEQ ID NO: 30) |
| PAMDA | Protospacer 2 | CTGATGGTCCATGTCTGTTACTC |
| library 2 | | (SEQ ID NO: 31) |
| Protospacer | PM0518 | /5Phos/CGATGTNNNNNGGAA |
| 1 top strand | | TCCCTTCTGCAGCACCTGGGCG |
| | | CAGGTCACGAGG |
| | | (SEQ ID NO: 32) |
| Protospacer | PM0519 | /5Phos/AATTCCTCGTGACCTG |
| 1 bottom | | CGCCCAGGTGCTGCAGAAGGG |
| strand | | ATTCCNNNNNACATCGCATG |
| | | (SEQ ID NO: 33) |
| Protospacer | PM0520 | /5Phos/CGATGTNNNNNCTGAT |
| 2 top strand | | GGTCCATGTCTGTTACTCGCGCA |
| | | GGTCACGAGG |
| | | (SEQ ID NO: 34) |
| Protospacer | PM0521 | /5Phos/AATTCCTCGTGACCTG |
| 2 bottom | | CGCGAGTAACAGACATGGACCAT |
| strand | | CAGNNNNNACATCGCATG |
| | | (SEQ ID NO: 35) |

The annealed double stranded fragments were ligated directly to a SphI and EcoRI digested pUC19 vector. The ligated protospacer constructs were used to transform XL1-blue Electro competent E. coil cells (Agilent) and recovered in 1 ml SOC media at 37° C. for 1 hr. Carbenicillin plates were used to check for the presence of the ligated products in the E. coli cells. The transformed E. coli cells were grown in LB broth supplemented (200 ml) with carbenicillin (50 mg/mL) for 16 hrs. The plasmids comprising the protospacer constructs were purified using Zymo midiprep kit. The plasmids/vectors were subjected to deep sequencing analysis to calculate the frequency of A/T/G/C at each PAM position using an Illumina. Miseq.

This method can be used to generate libraries for PAM determination using any protospacer oligonucleotide(s) of choice wherein the annealed oligonucleotides may comprise any appropriate restriction site selected so as to retain the full complement of PAM sequences in the library.

### Example 2

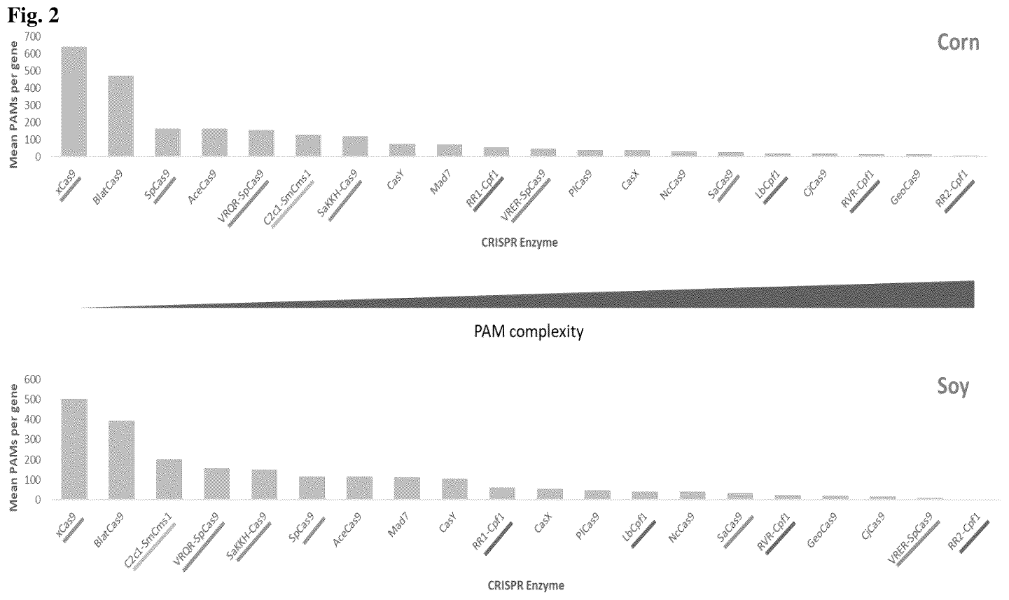

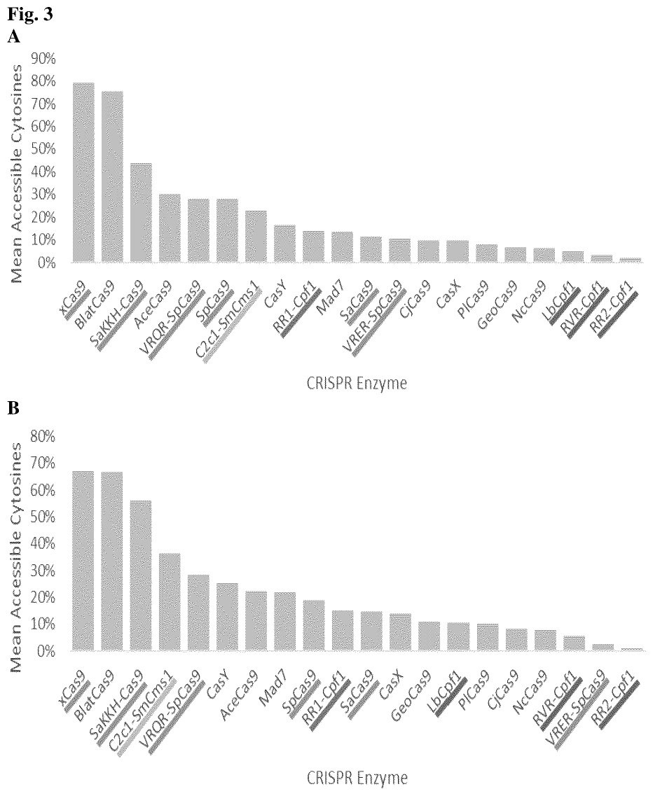

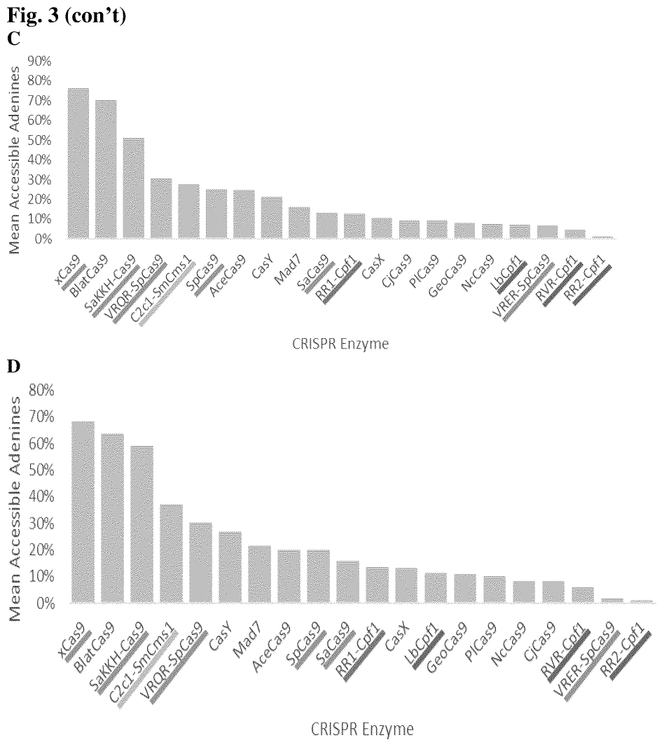

Lachnospiraceae bacterium ND2006 Cpf1 (LbCpf1) requires a highly specific protospacer adjacent motif (PAM). The ‘TTTV’ sequence occurs only about 1 in 85 bases in comparison to random nucleotides. This is contrast to the relative promiscuity of NGG for SpCas9 which occurs about 1 in 16 bases in random DNA, TTN for AaC2c1 which occurs about 1 in 16 bases in random DNA, and xCas9/Cas9-NG whose NG PAM requirements occur about 1 in 4 bases. Cpf1 PAMs are far less abundant in corn and soy genes than Cas9 PAMs (FIG. 2). Additionally, adenines and cytosines (the current targets for base editors) are far less accessible to LbCpf1 based on its strict PAM requirement (FIG. 3).

Such stringency as that shown in FIG. 3 for the CRISPR-Cas nucleases greatly reduces the potential targets and generation of new traits. The present invention is directed to generation of CRISPR-Cas nucleases, in particular, LbCpf1 (Cas12a) nucleases having an improved ratio of accessible PAM sequences (e.g., nucleases that has a PAM recognition site that occurs at a ratio of about 1:4 or better). Such engineered Cas12a PAM mutants may be used as nucleases (for NHEJ or HDR applications) or inactivated versions can be used as genomic recognition element in genome editing tools.

### PAMDA Assay

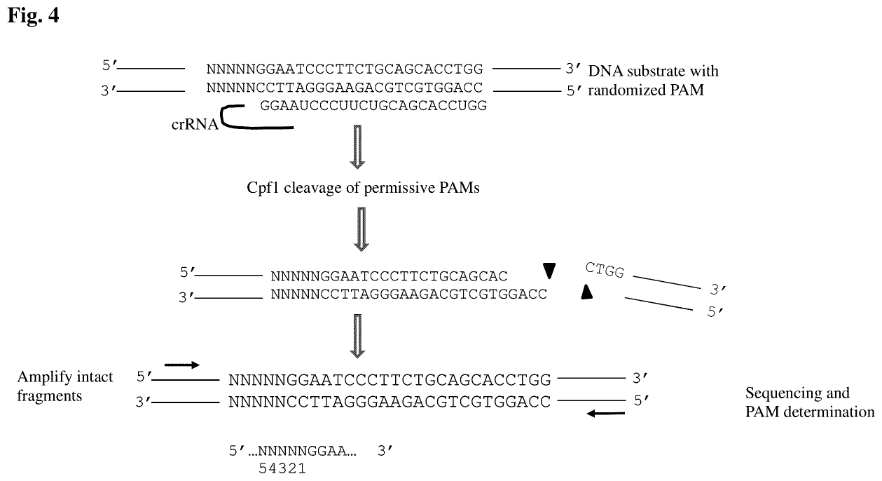

PAM Determination Assays (PAMDAs) are useful for testing PAM requirements for CRISPR enzymes with unknown PAM recognition. These are in vitro assays which take advantage of the ability of CRISPR-Cas nucleases to cleave target sequences only after successful PAM binding. Briefly, a library of DNA substrates with randomized PAM sequences are incubated with the CRISPR nuclease and then DNA is amplified via PCR. Only intact fragments (e.g., those not recognized by the nuclease) are amplified. Cleaved fragments (those that are recognized by the nuclease) are not amplified. The DNA from both the library that is exposed to the nuclease and a control library (not exposed to the nuclease) are sequenced. The two sets of sequencing results are compared to determine which sequences were cleaved, and thus, not present in the sequencing assemblies post exposure to the nuclease (see, as an example, FIG. 4). A modified PAMDA that uses multiple time points was used to determine PAM binding and subsequent cleavage.

### LbCpf1 Mutagenesis

One hundred and eighty six (186) point mutations (Table 2) were designed and individually tested in a PAMDA assay as described herein. Successful engineering may change the PAM recognition sequence generating novel PAM recognizing LbCpf1s or may relax PAM stringency resulting in a more promiscuous LbCpf1.

TABLE 2
Residues for substitution in SEQ ID NO: 1

| | | | | | | |
|---|---|---|---|---|---|---|
| K116R | K120R | K121S | D122R | E125R | T148H | T149A |
| K116N | K120H | K121T | D122K | E125K | T148S | T149C |
| | K120N | K121H | D122H | E125Q | T148A | T149S |
| | K120T | K121R | D122E | E125Y | T148C | T149G |
| | K120Y | K121G | D122N | | | T149H |
| | K120Q | K121D | | | | T149P |
| | | K121Q | | | | T149F |
| | | | | | | T149N |
| | | | | | | T149D |
| | | | | | | T149V |
| T152R | D156R | E159K | Q529N | G532D | D535N | K538R |
| T152K | D156K | E159R | Q529T | G532N | D535H | K538V |
| T152W | D156Y | E159H | Q529H | G532S | D535V | K538Q |
| T152Y | D156W | E159Y | Q529A | G532H | D535T | K538W |
| T152H | D156Q | E159Q | Q529F | G532F | D535S | K538Y |
| T152Q | D156H | | Q529G | G532K | D535A | K538F |
| T152E | D156I | | Q529S | G532R | D535W | K538H |
| T152L | D156V | | Q529P | G532Q | D535K | K538L |
| T152F | D156L | | Q529W | G532A | | K538M |
| | D156E | | Q529D | G532L | | K538C |
| | | | | G532C | | K538G |
| | | | | | | K538A |
| | | | | | | K538P |
| D541N | Y542R | L585G | K591W | M592R | K595R | V596T |
| D541H | Y542K | L585H | K591F | M592K | K595Q | V596H |
| D541R | Y542H | L585F | K591Y | M592Q | K595Y | V596G |
| D541K | Y542Q | | K591H | M592E | K595L | V596A |
| D541Y | Y542F | | K591R | M592A | K595W | |
| D541I | Y542L | | K591S | | K595H | |
| D541A | Y542M | | K591A | | K595E | |
| D541S | Y542P | | K591G | | K595S | |
| D541E | Y542V | | K591P | | K595D | |
| | Y542N | | | | K595M | |
| | Y542T | | | | | |
| S599G | K600R | K601R | Y616K | Y646R | W649H | |
| S599H | K600H | K601H | Y616R | Y646E | W649K | |
| S599N | K600G | K601Q | Y616E | Y646K | W649R | |
| S599D | | K601T | Y616F | Y646H | W649Y | |
| | | | Y616H | Y646Q | W649E | |
| | | | | Y646W | W649S | |
| | | | | Y646N | W649V | |
| | | | | | W649T | |

In addition to the individual mutations, combinations of mutations which alter the PAM recognition are combined and evaluated via PAMDA to provide a second generation of LbCpf1 mutations.

### Example 3

Three methods were used to test the 186 mutations:

(1) An in vitro method, known as a PAMDA assay (Kleinstiver et al. Nat Biotechnol 37:276-282 (2019)), which uses purified protein and a plasmid library to test each point mutation across the library. A depletion of a library member was scored using next generation sequencing (NGS). Depletions were calculated either to the library itself (to determine absolute activity against a particular PAM) or against cleavage by wild-type LbCas12a (to determine if the mutation imparted new PAM recognition as compared to wild-type).

(2) A bacterial method, known as PAM-SCANR (Leenay et al. Mol Cell 62:137-147 (2016)), which uses a library in Escherichia coli to test binding of Cas12a mutations to the 256 possible PAM NNNN variants. It does not test cleavage, just binding. Since the mutations made were nowhere near the catalytic region, binding is expected to reflect cleavage as well (verified later in the 293T assay). The advantage to PAM-SCANR is the ability to rapidly test not only point mutations, but combinations of amino acid point mutations in a quick and accurate way. This assay can be more stringent than in vitro cleavage assays.

(3) An INDEL assay in human HEK293T cells. This assay is provides valuable eukaryotic INDEL data. In order to get insertions and deletions in eukaryotes, a number of criteria have to be met: the CRISPR enzyme needs to be expressed and stable in the cell, the crRNA needs to be expressed and correctly processed, the protein:RNA complex needs to form, the complex needs to be stable, the complex needs to translocate in sufficient quantities into the nucleus, the target DNA needs to be accessible, the DNA has to be well-targeted by the particular guide-RNA design, and double-stranded breaks need to occur at a rate high enough to yield the occasional DNA repair mistake via an insertion or deletion (INDEL). This makes eukaryotic assays the most stringent assays in this study. A few dozen PAMs were tested for each of the 3 point mutants described below, rather than all 256 due to the experiment being low-throughput. Three different targets were chosen for each PAM-mutant combination to try to avoid false-negatives since often a particular guide is ineffective due to target accessibility.

### 1. Determining PAM Binding and Cleavage In Vitro

### Building a PAM Plasmid-Based Library

A DNA library consisting of 5 random nucleotides directly 5′ to a 23 nucleotide spacer sequence was prepared. LbCas12a is known to have a 4 nucleotide Protospacer Adjacent Motif (PAM), however we chose to use 5 random nucleotides rather than 4 to allow for replication within the experiment. The spacer sequence used was 5′-GGAATCCCTTCTGCAGCACCTGG (SEQ ID NO:30). The library contained the sequence 5?-NNNNNGGAATCCCTTCTGCAGCACCTGG (SEQ ID NO:36). Having 5 random nucleotides yield 1024 possible PAMs assayed in this library.

We used a novel way to generate this library. Rather than using a single randomized pool of PAM-spacer fusions and using a polymerase to generate the complimentary strand as has been previously described (Kleinstiver et al. Nat Biotechnol 37:276-282 (2019)), we chose a more direct method. Two 5′-phosphorylated sequences were synthesized:

(SEQ ID NO: 32)
5′phos/CGATGTNNNNNGGAATCCCTTCTGCAGCACCTGGGCGCAGGTC
ACGAGG
and
(SEQ ID NO: 35)
AATTCCTCGTGACCTGCGCCCAGGTGCTGCAGAAGGGATTCCNNNNNACA
TCGCATG/5′phos.

When heated and annealed, complimentary sequences between the two NNNNN sequences anneal and the resulting ends have overhangs corresponding to overhangs generated by SphI and EcoRI restriction endonucleases. The two oligonucleotides were annealed at an equal molar ratio in a thermal cycler at 95° C. for 5 min, and cooled for 0.1° C./sec to 25° C./room temperature.

The annealed double stranded fragments were ligated directly to SphI and EcoRI digested pUC19 vector. The ligated spacer constructs were used to transform XL1-blue Electro competent E. coli cells (Agilent) and recovered in 1 ml Super Optimal broth with glucose (SOC) media at 37° degree for 1 hr. A proportion of aliquot was plated on carbenicillin plates to check for the presence of the ligated products. The remaining transformed cells were grown in 200 ml Luria broth (LB) supplemented with 50 mg/mL carbenicillin for 16 hrs. The spacer plasmids were purified using plasmid midiprep kit (Zynto Research).

Verification of PAM library

The spacer vectors were subjected to deep sequencing analysis to calculate the frequency of A/T/G/C at each position of PAM using an Illumina MiSeq according to manufacturer's protocols. Briefly, 10 ng of DNA was used as template for PCR. Phasing gene specific forward and reverse PCR primers were designed to amplify across the target site. Amplicon libraries were generated using a two-step PCR method, where primary PCR with 5′ tails allow a secondary PCR to add Illumina i5 and i7 adapter sequences and barcodes for sorting multiplexed samples. PCR amplifications were performed using the following parameters: 98° C. for 30 s; 25 cycles for PCR1 and 8 cycles for PCR2 (98° C. 10 s, 55° C. 20 s, 72° C. 30 s); 72° C. for 5 min; hold at 12° C. The PCR reactions were performed with Q5 High-Fidelity DNA Polymerase (New England BioLabs, Beverly, MA, United States). The secondary PCR amplicon samples were individually purified using AMPure XP beads according to manufacturer's instruction (Beckman Coulter, Brea, CA, United States); all purified samples were quantified using a plate reader, pooled with an equal molar ratio, and run on AATI fragment analyzer (Agilent Technologies, Palo Alto, CA, United States). The pooled amplicon libraries were sequenced on an Illumina MiSeq (2×250 paired end) using a MiSeq Reagent kit v2 (Illumina, San Diego, CA, United States).

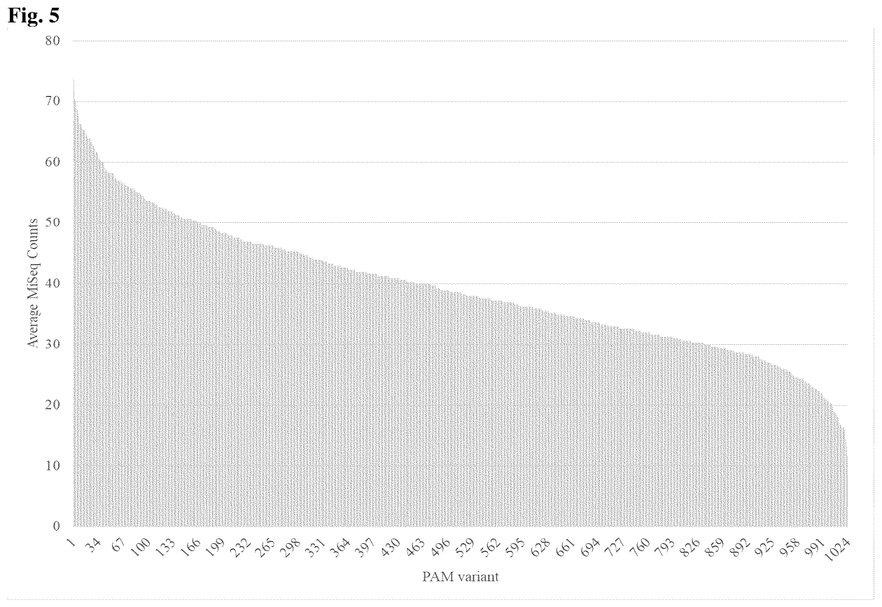

Three separate reads were generated for the library and averaged. The resulting 1024 library members had an average read count of 39 reads and a standard deviation of 11.9 reads. The maximum number of average reads for any PAM sequence was 74 and the minimum was 12. PAM counts followed a normal distribution (FIG. 5)

### Cloning LbCas12a Mutations

A DNA cassette composed of an LbCas12a sequence, followed by a nucleoplasmin NLS and a 6× histidine tag was synthesized (GeneWiz) (SEQ ID NO:52) and cloned into a pET28a vector between NcoI and XhoI, generating pWISE450 (SEQ ID NO:53). An additional glycine was added to the sequence between Met-1 and Ser-2 to facilitate cloning. Numbering throughout this document excludes this extra glycine. Then, 186 different amino acid point mutations (Table 2) were made using a similar strategy yielding 186 different plasmid vectors.

### Expression and Purification of LbCas12a Mutations

Glycerol stocks of each mutant in BL21 Star (DE3) cells (ThermoFisher Scientific) were used to inoculate one mL of media with 50 μg/mL of kanamycin in a 24-well block. Cultures were sealed with AirPore tape sheets (Qiagen) and grown overnight at 37° C. with shaking. The next morning four mL of ZYP autoinduction media with kanamycin were inoculated with 100 μL of overnight culture and incubated at 37° C. with shaking until OD 600 nm range of 0.2-0.5. The temperature was reduced to 18° C. and cultures were grown overnight for protein expression. Cells were harvested by centrifugation and pellets were stored at −80° C.

The following buffers were used for cell lysis and purification. A lysis buffer comprising non-ionic detergents, lysing agents, reducing agents, protease inhibitors, buffers, and salts. This solution was able to lyse bacteria, reduce viscosity, and allow for downstream purification of enzymes free from interfering nucleases. Buffer A composed of 20 mM Hepes-KOH pH 7.5, 0.5 M NaCl, 10% glycerol, 2 mM TCEP and 10 mM Imidazole pH 7.5. Buffer B was the same as buffer A but also included Imidazole at 20 mM. Buffer C contained 20 mM Hepes-KOH pH 7.5, 150 mM NaCl, 10% glycerol, 0.5 mM TCEP, and 200 mM Imidazole pH 7.5.

Purification was performed using a multi-well format. Two stainless steel 5/32″ BBs were added to all wells containing cell pellets. Pellets were resuspended in 0.5 mL cold lysis buffer and incubated at room temperature for 30 minutes with orbital mixing. The crude lysates, 0.5 mL, were added to pre-equilibrated His MultiTrap™ plates (Cytiva LifeSciences). The plates were incubated for five minutes at room temperature to allow for protein binding. The remaining steps were performed per manufacturer's instructions. Briefly, plates were washed two times with 0.5 mL buffer A followed by one wash with 0.5 mL buffer B before eluting in 0.2 mL of buffer C. Protein concentrations were determined using Pierce™ Coomassie Plus (Bradford) Assay Reagent. The protein eluates were stored at 4° C.

Test Cleavage of PAM Library by wtLbCas12a

A pre-test was performed to assess three aspects of the experiment: ensure the experiment was free from non-specific nucleases, ensure that there was depletion at NTTTV PAMs from the library upon addition of crRNA guides, and to see the extent of depletion at 15 minutes for a spiked sample of CTTTA.

Reaction conditions for the test depletions were: 27 μL total volume of containing nuclease-free water, 3 μL NEB buffer 2.1 (New England Biolabs), 3 μL stock of the crRNA at 300 nM (5′-AAUUUCUACUAAGUGUAGAUGGAAUCCCUUCUGCAGCACCUGG-3′ (SEQ ID NO:62), Synthego Corporation), and 1 μL of the purified wtLbCas12a at 1 μM stock were incubated at room temperature for 10 minutes. 3 μL of a 10 ng/RL stock was added to start the reaction. The library was either added as-is or 1 μL of the CTTTA-containing plasmid was added first at 0.75 ng/μL. Total volumes were all 30 μL. The reaction was incubated at 37° C. for 15 minutes.

Table 3 provides the results of the experiment. The library counts for TTTV sequences were between 219 and 515 counts (column 2), adding the wildtype protein which was purified as described in the absence of a crRNA did not result in depletion of the library members (column 3), addition of a crRNA and the protein resulted in depletion of all the NTTTV containing PAMs (column 4), spiking the library with CTTTA resulted in approximately 35 times as many CTTTA NGS counts (column 5), addition of wtLbCas12a and the crRNA resulted in a depletion of all library members, including CTTTA decreasing from 10,776 to 193 counts. Also shown are NACGA PAM-containing library members which show no depletion under the conditions tested, as is expected since ACGA is not a PAM recognized by LbCas12a. Thus, as shown in Table 3, NTTTV PAM library members are efficiently cleaved and depleted by wtLbCas12a, whereas PAMs not recognized by wtLbCas12a (NACGA) are not.

TABLE 3
PAM library cleavage and depletion.

| | | | | | Library |
|---|---|---|---|---|---|
| | | | Library + | Library | spiked with |
| | Library | Library + | wtLbCas12a + | spiked with | CTTTA + |
| | member | wtLbCas12a | crRNA | CTTTA | wtLbCas12a + |
| PAM | NGS count | NO crRNA | (15 min) | (15 min) | crRNA |
| (substrate) | | | | | |
| ATTTG | 314 | 336 | 13 | 294 | 30 |
| CTTTC | 219 | 245 | 13 | 225 | 15 |
| ATTTC | 317 | 392 | 22 | 294 | 22 |
| ATTTA | 515 | 490 | 37 | 396 | 37 |
| TTTTC | 359 | 382 | 33 | 286 | 28 |
| TTTTA | 489 | 666 | 46 | 446 | 32 |
| GTTTG | 330 | 438 | 34 | 297 | 30 |
| GTTTA | 395 | 492 | 41 | 378 | 31 |
| CTTTG | 299 | 333 | 35 | 263 | 28 |
| TTTTG | 348 | 430 | 41 | 343 | 24 |
| CTTTA | 293 | 379 | 35 | 10776 | 193 |
| GTTTC | 299 | 349 | 39 | 269 | 14 |
| (non- | | | | | |
| substrate) | | | | | |
| AACGA | 215 | 271 | 237 | 197 | 211 |
| GACGA | 273 | 282 | 274 | 251 | 282 |
| CACGA | 264 | 294 | 253 | 194 | 235 |
| TACGA | 302 | 268 | 255 | 225 | 243 |

The results in Table 3 show that (1) effective and nuclease-free purification of wtLbCAs12a was achieved, (2) the library can be depleted under conditions tested for members containing a PAM-substrate, (3) the depletion results upon the addition of a spacer-targeting crRNA, and (4) the enzyme-crRNA complex is in vast-excess of the individual library members since large amounts of CTTTA substrate does not alter the depletion of the substrates.

### Cleavage of PAM Library by LbCas12a Mutants

Identical reaction conditions were tested for each of the 186 PAM mutations as were shown in the test example of wtLbCas12a. Three time points were chosen for each mutation: 75, 435, and 900 seconds at 37° C. Multiple library-only controls were included. The products were subjected to Illumina HiSeq analysis (Genewiz). The data are reported in Table 4.

### Absolute Depletion Scores Processing

We observed little difference in depletion between the four possibilities for any 5 nucleotide PAM. In other words: ANNNN, CNNNN, GNNNN, and TNNNN for any 4 nucleotide sequence had similar PAM depletions. This was in-line with what we observed in the wildtype LbCAs12a experiment which showed NTTTV sequences were all depleted at a similar amount regardless if the N was A, C, G, or T (Table 3). Secondly, we observed the three time points of 75, 435, and 900 seconds all had similar depletions. This indicated that the reaction was nearly complete after just 75 seconds at 37° C. We, therefore, were able to average all four 4nt PAMs from the 5nt library and average all three time points, effectively resulting in 12 data points for each PAM. We then took that average and divided it by the median library values for each PAM. This gave us a depletion score for each 4 nucleotide PAM against all 186 mutants. A depletion of 10 indicates 90% of the 4 parental plasmid library members with that 4 nt PAM were depleted, while a score of 20 indicates 95% depletion.

The depletion score for wildtype LbCas12a was 9.2 for TTTV sequences, so any mutant which cleaved a PAM at or better than this 9.2 score was considered efficacious using wild type as a benchmark. For example, Table 4 shows the mutant LbCas12a-K595Y depleting in vitro 45 different PAM 4mers from the library at or better than wtLbCas12a cleaves TTTV-containing sequences. This analysis was used to score each of the 186 mutations to determine in vitro PAM recognition and cleavage by each of the mutants. The data containing recognition sequences are shown in Table 4, which shows LbCas12a-K595Y PAMDA depletion scores at or better than wtLbCas12a scores against TTTV-containing sequences of 9.2.

TABLE 4
PAM depletion data

| Mutant | 4mer | Depletion |
|---|---|---|
| D122H | TCCC | 18.76 |
| D122H | ATTC | 17.15 |
| D122H | GTTC | 16.7 |
| D122H | TCCG | 15.92 |
| D122H | TTCA | 14.95 |
| D122H | TCGC | 14.8 |
| D122H | TTCT | 14.68 |
| D122H | CCCC | 14.61 |
| D122H | CTTG | 14.37 |
| D122H | GTTG | 14.14 |
| D122H | ATTA | 13.66 |
| D122H | TCTA | 12.99 |
| D122H | CCTG | 12.96 |
| D122H | GTTA | 12.87 |
| D122H | CCTA | 12.77 |
| D122H | TTCG | 12.68 |
| D122H | CCTC | 12.5 |
| D122H | ATTG | 12.43 |
| D122H | TTGA | 12.14 |
| D122H | TTAC | 11.87 |
| D122H | GCGC | 11.86 |
| D122H | CTTA | 11.78 |
| D122H | CTCA | 11.77 |
| D122H | TCTC | 11.75 |
| D122H | TCCA | 11.74 |
| D122H | CCGC | 11.64 |
| D122H | TCGG | 11.3 |
| D122H | CCCG | 11.05 |
| D122H | CTTC | 11.01 |
| D122H | TCAC | 10.9 |
| D122H | TTTT | 10.85 |
| D122H | TTTC | 10.27 |
| D122H | TTTA | 10.18 |
| D122H | ACTC | 10.17 |
| D122H | GCTC | 10.01 |
| D122H | TTGC | 9.8 |
| D122H | TTCC | 9.8 |
| D122H | TTGG | 9.66 |
| D122H | CCCA | 9.34 |
| D122K | TCCC | 13.07 |
| D122K | TCCG | 13.02 |
| D122K | TCTA | 12.97 |
| D122K | GTTA | 11.7 |
| D122K | TCTC | 11.54 |
| D122K | CCCC | 11.24 |
| D122K | TCTG | 10.77 |
| D122K | GTTC | 10.77 |
| D122K | TTAC | 10.53 |
| D122K | ATTC | 10.5 |
| D122K | CCGC | 10.33 |
| D122K | TTCT | 10.1 |
| D122K | CTCC | 10.09 |
| D122K | GTTG | 10.04 |
| D122K | ATTG | 10.02 |
| D122K | TCGC | 9.84 |
| D122K | TCGG | 9.84 |
| D122K | ATTA | 9.76 |
| D122K | TTCA | 9.46 |
| D122K | CTTG | 9.44 |
| D122K | CTTC | 9.39 |
| D122K | CCTA | 9.34 |
| D122K | TTTT | 9.23 |
| D122K | TCCA | 9.11 |
| D122K | TCAC | 8.97 |
| D122K | GCTA | 8.88 |
| D122K | TTCC | 8.77 |
| D122K | GCGC | 8.76 |
| D122K | CCTC | 8.6 |
| D122K | TTGC | 8.56 |
| D122K | TTCG | 8.43 |
| D122K | TTTC | 8.41 |
| D122N | TCCC | 11.48 |
| D122N | TTTT | 11 |
| D122N | TCTA | 10.38 |
| D122N | GTTG | 10.31 |
| D122N | ATTC | 10.27 |
| D122N | CCCC | 10.22 |
| D122N | TTCA | 10.16 |
| D122N | TCCG | 10.02 |
| D122N | TTCT | 9.86 |
| D122N | CTTG | 9.79 |
| D122N | TCGC | 9.72 |
| D122N | TCTG | 9.66 |
| D122N | ATTA | 9.62 |
| D122N | CTTA | 9.61 |
| D122N | CTTC | 9.49 |
| D122N | TCCA | 9.46 |
| D122N | TTCC | 9.44 |
| D122N | TTGC | 9.39 |
| D122N | TTAC | 9.36 |
| D122N | GTTC | 9.22 |
| D122N | CCTA | 9.18 |
| D122N | CCTC | 9.16 |
| D122N | GTTA | 9.14 |
| D122N | TTCG | 9.04 |
| D122N | TCTC | 9.01 |
| D122N | TTGG | 8.97 |
| D122N | ATTG | 8.7 |
| D122R | TCCC | 10.09 |
| D122R | GTTC | 9.87 |
| D122R | CCCC | 9.85 |
| D122R | ATTG | 9.82 |
| D122R | TTGC | 9.8 |
| D122R | GTTA | 9.53 |
| D122R | TTAC | 8.94 |
| D122R | TTCG | 8.87 |
| D122R | CTTA | 8.8 |
| D122R | GCGC | 8.73 |
| D122R | TTCT | 8.6 |
| D122R | CCGC | 8.6 |
| D122R | CCTC | 8.53 |
| D122R | ATTA | 8.25 |
| D122R | TCAC | 8.17 |
| D122R | TTTT | 8.09 |
| D122R | GTTG | 7.81 |
| D156E | TTCG | 14.53 |
| D156E | TTTT | 14.35 |
| D156E | CTTA | 13.86 |
| D156E | TTGC | 13.64 |
| D156E | ATTC | 13.58 |
| D156E | TTCT | 13.51 |
| D156E | TTCA | 13.24 |
| D156E | TCTG | 13.06 |
| D156E | TCCC | 13.05 |
| D156E | CTTG | 13 |
| D156E | TTCC | 12.52 |
| D156E | ATTG | 12.43 |
| D156E | TCTA | 12.35 |
| D156E | TTAC | 12.06 |
| D156E | TCTC | 11.97 |
| D156E | GTTG | 11.9 |
| D156E | ATTA | 11.88 |
| D156E | CCCG | 11.88 |
| D156E | CTTC | 11.51 |
| D156E | TTTC | 11.46 |
| D156E | CCTG | 11.34 |
| D156E | CCTA | 11.19 |
| D156E | GTTA | 11.16 |
| D156E | GTTC | 10.73 |
| D156E | TTTG | 10.61 |
| D156E | TTTA | 10.25 |
| D156E | CCCC | 10.16 |
| D156E | CCTC | 9.91 |
| D156E | CTCC | 9.83 |
| D156E | CTCA | 9.57 |
| D156E | TTGG | 8.81 |
| D156E | GCTC | 8.57 |
| D156H | CCTG | 13.3 |
| D156H | TCTG | 12.9 |
| D156H | TTCC | 12.22 |
| D156H | TTAC | 12.08 |
| D156H | ATTC | 12.04 |
| D156H | CCTC | 11.12 |
| D156H | CCCA | 10.92 |
| D156H | TCGC | 10.89 |
| D156H | TTGC | 10.87 |
| D156H | GCTA | 10.68 |
| D156H | GTTG | 10.6 |
| D156H | CCCG | 10.37 |
| D156H | CCTA | 10.3 |
| D156H | GTTA | 10.27 |
| D156H | GCTC | 10.15 |
| D156H | CTCC | 10.03 |
| D156H | GCGC | 10 |
| D156H | ATTA | 10 |
| D156H | TCCG | 9.99 |
| D156H | TCAC | 9.98 |
| D156H | TCTA | 9.95 |
| D156H | TCTC | 9.74 |
| D156H | TTCT | 9.69 |
| D156H | GTTC | 9.51 |
| D156H | CCGC | 9.45 |
| D156H | TTCA | 9.4 |
| D156H | CCCC | 9.28 |
| D156H | TCGA | 9.14 |
| D156H | TTTT | 9.13 |
| D156H | GCCC | 9.1 |
| D156H | TCCA | 9.05 |
| D156H | ACTC | 8.98 |
| D156H | TTCG | 8.89 |
| D156H | ATTG | 8.88 |
| D156H | ACTA | 8.4 |
| D156H | CTCG | 8.13 |
| D156I | CCGC | 10.23 |
| D156I | TCAG | 9.16 |
| D156I | TCGA | 8.68 |
| D156I | ACCC | 8.52 |
| D156I | GCGC | 8.42 |
| D156I | ATTC | 8.37 |
| D156I | TCCC | 8.33 |
| D156I | ACTC | 8.31 |
| D156I | GCCC | 8.05 |
| D156I | GTTG | 7.84 |
| D156K | TTCC | 12.11 |
| D156K | TTCT | 12.02 |
| D156K | ATTA | 11.58 |
| D156K | CCTG | 11.51 |
| D156K | CCTA | 11.41 |
| D156K | TCCC | 11.39 |
| D156K | TCCG | 11.32 |
| D156K | GTTC | 11.24 |
| D156K | TTGA | 11.23 |
| D156K | ATTC | 11.16 |
| D156K | ATTG | 10.87 |
| D156K | GCTC | 10.8 |
| D156K | GTTA | 10.77 |
| D156K | TTGC | 10.71 |
| D156K | CTTC | 10.65 |
| D156K | TTCA | 10.57 |
| D156K | TCTG | 10.41 |
| D156K | CCCC | 10.25 |
| D156K | TCGC | 10.15 |
| D156K | CTCC | 10 |
| D156K | TCTA | 9.99 |
| D156K | TTCG | 9.98 |
| D156K | TTAC | 9.83 |
| D156K | CCTC | 9.73 |
| D156K | TCCA | 9.65 |
| D156K | TTTT | 9.64 |
| D156K | ACTA | 9.56 |
| D156K | GTTG | 9.44 |
| D156K | TCTC | 9.37 |
| D156K | CCCG | 9.21 |
| D156K | TCAC | 9.18 |
| D156K | TTTC | 9.13 |
| D156K | CTTG | 9.05 |
| D156K | CTCA | 9 |
| D156K | TTTG | 8.92 |
| D156K | GCTG | 8.91 |
| D156K | TTGG | 8.53 |
| D156K | ACTC | 8.42 |
| D156L | GTTG | 15.01 |
| D156L | ATTC | 14.48 |
| D156L | CTTC | 14.39 |
| D156L | TCCC | 14.28 |
| D156L | ATTA | 13.69 |
| D156L | TCCG | 13.57 |
| D156L | CCCG | 13.52 |
| D156L | TTAC | 13.32 |
| D156L | CCTG | 13.25 |
| D156L | TCGC | 13.04 |
| D156L | TTCT | 12.9 |
| D156L | CCTC | 12.55 |
| D156L | ATTG | 12.52 |
| D156L | CCCC | 12.48 |
| D156L | TTGC | 12.37 |
| D156L | GTTC | 12.31 |
| D156L | ACTA | 12.29 |
| D156L | TCTC | 11.89 |
| D156L | TCTA | 11.73 |
| D156L | CCTA | 11.49 |
| D156L | TTTT | 11.47 |
| D156L | CTTA | 11.36 |
| D156L | CTTG | 11.36 |
| D156L | TTCG | 11.36 |
| D156L | TCTG | 11.07 |
| D156L | GTTA | 11.02 |
| D156L | CCCA | 10.93 |
| D156L | GCTA | 10.87 |
| D156L | TTTG | 10.8 |
| D156L | TTCC | 10.73 |
| D156L | TCCA | 10.63 |
| D156L | CTCC | 10.14 |
| D156L | TTCA | 10.07 |
| D156L | TTTC | 9.96 |
| D156L | CTCA | 9.82 |
| D156L | TCAC | 9.8 |
| D156L | TTTA | 9.69 |
| D156L | ACTG | 9.67 |
| D156L | GCGC | 9.08 |
| D156L | CCGC | 8.68 |
| D156L | GCTC | 8.48 |
| D156L | TCAG | 8.46 |
| D156L | CTCG | 8.35 |
| D156Q | GTTC | 15.61 |
| D156Q | TCCC | 15.02 |
| D156Q | ATTC | 14.2 |
| D156Q | TCCA | 14.13 |
| D156Q | TCTA | 13.69 |
| D156Q | CCTA | 13.4 |
| D156Q | CCTG | 13.17 |
| D156Q | ATTG | 12.94 |
| D156Q | TCTG | 12.76 |
| D156Q | TCTC | 12.71 |
| D156Q | CTCC | 12.58 |
| D156Q | CCCC | 12.54 |
| D156Q | CTTG | 12.5 |
| D156Q | GTTG | 12.46 |
| D156Q | CCTC | 11.6 |
| D156Q | CTCA | 11.56 |
| D156Q | TTTT | 11.44 |
| D156Q | CCCG | 11.41 |
| D156Q | ACTG | 11.38 |
| D156Q | ATTA | 11.35 |
| D156Q | GTTA | 11.2 |
| D156Q | CTTA | 11.06 |
| D156Q | TTCA | 10.96 |
| D156Q | CTTC | 10.86 |
| D156Q | TTCG | 10.7 |
| D156Q | TTGC | 10.65 |
| D156Q | TTAC | 10.6 |
| D156Q | ACTA | 10.58 |
| D156Q | TCGC | 10.58 |
| D156Q | TTCT | 10.47 |
| D156Q | TTGA | 10.35 |
| D156Q | TCCG | 10.31 |
| D156Q | GCTA | 10.26 |
| D156Q | GCTG | 10.21 |
| D156Q | TTTG | 10.17 |
| D156Q | GCTC | 10.12 |
| D156Q | TTTC | 10.01 |
| D156Q | TCAC | 9.87 |
| D156Q | CCCA | 9.86 |
| D156Q | CTCG | 9.78 |
| D156Q | GCGC | 9.58 |
| D156Q | TTCC | 9.43 |
| D156Q | TTTA | 9.38 |
| D156Q | CTTT | 9.36 |
| D156Q | TTAG | 9.1 |
| D156R | CCTG | 11.46 |
| D156R | GCTA | 11.19 |
| D156R | TTCT | 11.15 |
| D156R | CCGC | 11.01 |
| D156R | CCTC | 10.98 |
| D156R | TCCA | 10.93 |
| D156R | CCCC | 10.57 |
| D156R | TCTG | 10.53 |
| D156R | TTGC | 10.34 |
| D156R | CTTC | 9.92 |
| D156R | TCGC | 9.46 |
| D156R | ACTA | 9.41 |
| D156R | GTTG | 9.21 |
| D156R | TCTA | 9.19 |
| D156R | TTTT | 9.1 |
| D156R | TCCC | 8.96 |
| D156R | CTCA | 8.63 |
| D156W | ACTA | 13.47 |
| D156W | CCTG | 12.62 |
| D156W | CCCA | 11.99 |
| D156W | GTTG | 11.77 |
| D156W | TCTG | 11.72 |
| D156W | CCTA | 11.4 |
| D156W | CCCG | 11.4 |
| D156W | GCTA | 11.28 |
| D156W | TCGC | 11.27 |
| D156W | CCTC | 11.21 |
| D156W | CCCC | 11.16 |
| D156W | TTGC | 10.91 |
| D156W | TCCG | 10.66 |
| D156W | ATTG | 10.65 |
| D156W | GTTC | 10.5 |
| D156W | TCCA | 10.43 |
| D156W | TCTC | 10.34 |
| D156W | TCTA | 10.25 |
| D156W | TTCC | 10.18 |
| D156W | TTCT | 10.1 |
| D156W | GTTA | 10.08 |
| D156W | TTCG | 9.92 |
| D156W | TTTT | 9.65 |
| D156W | CTCA | 9.63 |
| D156W | CTTC | 9.54 |
| D156W | TCCC | 9.49 |
| D156W | ATTA | 9.47 |
| D156W | CTTG | 9.45 |
| D156Y | TCTG | 12.9 |
| D156Y | ATTC | 12.41 |
| D156Y | TCTA | 12.32 |
| D156Y | TCCC | 11.75 |
| D156Y | TCTC | 11.68 |
| D156Y | ATTG | 10.93 |
| D156Y | CTTA | 10.52 |
| D156Y | TTCC | 10.34 |
| D156Y | GTTG | 10.31 |
| D156Y | CTTC | 10.17 |
| D156Y | TTCT | 10.02 |
| D156Y | TTGC | 9.75 |
| D156Y | TTTA | 9.64 |
| D156Y | TTCG | 9.59 |
| D535A | TTCC | 17.75 |
| D535A | TTCA | 16.69 |
| D535A | CTTG | 16.57 |
| D535A | CTTA | 15.51 |
| D535A | TCTC | 15.39 |
| D535A | TCGC | 15.38 |
| D535A | TCTA | 15.02 |
| D535A | TTGC | 14.6 |
| D535A | TCCC | 14.6 |
| D535A | TTTA | 14.59 |
| D535A | TTAC | 14.56 |
| D535A | TTTT | 13.92 |
| D535A | TTCG | 13.69 |
| D535A | TTTC | 12.57 |
| D535A | CTTC | 12.57 |
| D535A | TCTG | 12.5 |
| D535A | TCCG | 12.08 |
| D535A | TTTG | 11.96 |
| D535A | ATTA | 11.71 |
| D535A | TTAG | 11.43 |
| D535A | CCCC | 11.28 |
| D535A | ATTG | 11.06 |
| D535A | TTGA | 10.6 |
| D535A | TTCT | 10.56 |
| D535A | GCGC | 10.49 |
| D535A | ATTC | 10.41 |
| D535A | TCCA | 10.31 |
| D535A | GTTG | 10.15 |
| D535A | TCAC | 9.7 |
| D535A | GTTC | 8.85 |
| D535A | CCCG | 8.33 |
| D535A | TCAG | 8.01 |
| D535H | TCAC | 12.82 |
| D535H | TTCA | 11.72 |
| D535H | TCCG | 11.42 |
| D535H | TTCG | 11.3 |
| D535H | TCTA | 11.27 |
| D535H | TCTC | 10.82 |
| D535H | CCCC | 10.68 |
| D535H | TTCT | 10.51 |
| D535H | TTTT | 10.43 |
| D535H | TCAG | 10.24 |
| D535H | ATTA | 10.22 |
| D535H | ATTG | 10.1 |
| D535H | TTAC | 10.04 |
| D535H | TCCC | 10.02 |
| D535H | TTCC | 9.97 |
| D535H | CTTC | 9.97 |
| D535H | TCGC | 9.95 |
| D535H | TTGC | 9.74 |
| D535H | TTTG | 9.52 |
| D535H | CCTA | 9.5 |
| D535H | CTTA | 9.28 |
| D535H | CCAG | 9.27 |
| D535H | TCTG | 9.24 |
| D535H | TTTA | 9.15 |
| D535H | TTGG | 8.73 |
| D535H | CCAC | 8.4 |
| D535H | GCAC | 8.08 |
| D535K | TTTA | 14.65 |
| D535K | TTTC | 13.64 |
| D535K | TTTG | 13.58 |
| D535K | TTGC | 11.64 |
| D535K | TTAC | 10.49 |
| D535N | TTGC | 10.94 |
| D535N | TTAG | 10.83 |
| D535N | TTCC | 10.61 |
| D535N | TTTC | 10.54 |
| D535N | TTTT | 10.52 |
| D535N | TCGC | 10.51 |
| D535N | TTAC | 10.39 |
| D535N | TTTA | 10.34 |
| D535N | TCAC | 10.22 |
| D535N | TTTG | 10.01 |
| D535N | TTAA | 9.04 |
| D535N | TCAG | 8.94 |
| D535N | TCCC | 8.46 |
| D535N | TTGG | 7.82 |
| D535S | TTCG | 12.34 |
| D535S | TCTG | 12.09 |
| D535S | CTTG | 12.07 |
| D535S | CTTA | 11.85 |
| D535S | TCTC | 11.41 |
| D535S | TTTA | 10.73 |
| D535S | TCCC | 10.7 |
| D535S | TTAC | 10.6 |
| D535S | TTAG | 10.49 |
| D535S | TTCC | 10.47 |
| D535S | TCAG | 10.46 |
| D535S | TCTA | 10.4 |
| D535S | TTCA | 10.36 |
| D535S | TCGC | 10.31 |
| D535S | ATTA | 10.14 |
| D535S | TTGA | 10.13 |
| D535S | TTTT | 10.1 |
| D535S | TCAC | 10.07 |
| D535S | TTTC | 9.76 |
| D535S | CCCC | 9.75 |
| D535S | TTGC | 9.72 |
| D535S | CCGC | 9.66 |
| D535S | ATTG | 9.64 |
| D535S | GTTG | 9.6 |
| D535S | TTCT | 9.51 |
| D535S | TTTG | 9.4 |
| D535S | CCTG | 9.34 |
| D535S | CCCG | 9.18 |
| D535S | TCCA | 9.11 |
| D535S | TTGG | 9.02 |
| D535S | TTAA | 8.98 |
| D535T | CTTA | 14.25 |
| D535T | TTCG | 13.61 |
| D535T | TTTT | 13.51 |
| D535T | TCCC | 12.73 |
| D535T | TCTA | 12.41 |
| D535T | CTTG | 12.36 |
| D535T | TCTG | 11.75 |
| D535T | TTGC | 11.65 |
| D535T | TCCG | 11.65 |
| D535T | TCTC | 11.38 |
| D535T | TCGC | 11.38 |
| D535T | TTAC | 11.38 |
| D535T | TTTA | 11.34 |
| D535T | TTTG | 11.25 |
| D535T | CTTC | 11.2 |
| D535T | TTGG | 10.8 |
| D535T | TTCT | 10.42 |
| D535T | TCAC | 10.06 |
| D535T | TCAG | 9.75 |
| D535T | TTTC | 9.07 |
| D535T | GTTA | 8.61 |
| D535T | CTCC | 8.06 |
| D535V | TTTG | 15.43 |
| D535V | TTTA | 13.9 |
| D535V | TTTC | 12.89 |
| D535V | TTGC | 9.81 |
| D535V | TTTT | 9.77 |
| D541A | ATTG | 9.99 |
| D541E | CTTC | 15.41 |
| D541E | TTTG | 14.24 |
| D541E | TTTT | 14.15 |
| D541E | CTTA | 13.23 |
| D541E | TTTC | 12.78 |
| D541E | CTTG | 11.68 |
| D541E | TTCA | 11.56 |
| D541E | TTCC | 11.02 |
| D541E | TTTA | 10.69 |
| D541E | CCGC | 10.13 |
| D541E | TCTA | 9.86 |
| D541E | TCTC | 9.77 |
| D541E | TCCG | 9.75 |
| D541E | TCCC | 9.37 |
| D541E | TTGC | 9.33 |
| D541E | TTCG | 9.03 |
| D541E | CCCC | 8.91 |
| D541E | TTAC | 8.69 |
| D541E | CCTG | 8.3 |
| D541E | ATTG | 8.18 |
| D541E | CCTA | 7.96 |
| D541E | GTTA | 7.84 |
| D541H | TTCG | 8.67 |
| D541H | CTTC | 8.58 |
| D541I | TTTC | 11.44 |
| D541I | TTTG | 10 |
| D541I | CTTA | 9.11 |
| D541I | TTTT | 8.7 |
| D541N | TCCC | 8.36 |
| D541R | TTTG | 8.99 |
| D541Y | CTTA | 10.32 |
| D541Y | TTTC | 9.36 |
| D541Y | CTTC | 8 |
| E125K | TTGC | 10.22 |
| E125K | TCTC | 9.44 |
| E125K | ATTG | 9.32 |
| E125K | TTAC | 9.11 |
| E125K | CCCC | 8.94 |
| E125K | TTCG | 8.93 |
| E125K | CTTC | 8.83 |
| E125K | TCCC | 8.81 |
| E125K | TTCT | 8.62 |
| E125K | TTTA | 8.56 |
| E125K | TTTT | 8.43 |
| E125K | TTGA | 8.25 |
| E125K | TCGC | 8.15 |
| E125K | ATTC | 8.04 |
| E125Q | TTCA | 12.74 |
| E125Q | CTTC | 12.55 |
| E125Q | CTTA | 12.14 |
| E125Q | TCCC | 11.7 |
| E125Q | TTCG | 11.66 |
| E125Q | TCTA | 11.06 |
| E125Q | TTGC | 10.88 |
| E125Q | ATTC | 10.84 |
| E125Q | ATTA | 10.53 |
| E125Q | TTTG | 10.51 |
| E125Q | TTTT | 10.08 |
| E125Q | CCCC | 10.04 |
| E125Q | TCTC | 10.03 |
| E125Q | CCCG | 9.96 |
| E125Q | TCCA | 9.94 |
| E125Q | TTGG | 9.92 |
| E125Q | TTCC | 9.88 |
| E125Q | TCCG | 9.84 |
| E125Q | TTCT | 9.61 |
| E125Q | CTTG | 9.55 |
| E125Q | ATTG | 9.45 |
| E125Q | GTTG | 9.29 |
| E125Q | TCTG | 9.17 |
| E125Q | TTTA | 9.1 |
| E125Q | TTAC | 8.56 |
| E125Q | TCGC | 8.55 |
| E125Q | TCAC | 8.5 |
| E125Q | CCTG | 8.42 |
| E125Q | CTCG | 8.25 |
| E125R | CCCG | 11.21 |
| E125R | TCCC | 8.35 |
| E125R | TCCA | 8.03 |
| E125Y | TTCG | 11.77 |
| E125Y | TTGC | 10.69 |
| E125Y | CTTC | 9.94 |
| E125Y | CCCC | 9.82 |
| E125Y | TTTT | 9.39 |
| E159K | TTCT | 12.66 |
| E159K | CCCC | 12.33 |
| E159K | ATTG | 11.53 |
| E159K | CTTG | 11.38 |
| E159K | CTTA | 11.24 |
| E159K | TCTC | 11.21 |
| E159K | ATTC | 11.05 |
| E159K | TCCC | 11.04 |
| E159K | TTCG | 10.86 |
| E159K | TTCA | 10.67 |
| E159K | TCTA | 10.64 |
| E159K | TTGC | 10.56 |
| E159K | CTTC | 10.36 |
| E159K | TCTG | 10.17 |
| E159K | TTAC | 10.12 |
| E159K | TTCC | 9.92 |
| E159K | TCGC | 9.78 |
| E159K | GTTA | 9.69 |
| E159K | TCCG | 9.67 |
| E159K | CCTG | 9.31 |
| E159K | GTTG | 9.04 |
| E159K | GCCC | 8.85 |
| E159K | CCTC | 8.85 |
| E159K | GCGC | 8.65 |
| E159K | ACTC | 8.23 |
| E159Q | TTTT | 11.37 |
| E159Q | TTCG | 10.89 |
| E159Q | CTTA | 10.51 |
| E159Q | TTGC | 10.5 |
| E159Q | TTTC | 10.15 |
| E159Q | TTCC | 9.95 |
| E159Q | CTTG | 9.91 |
| E159Q | TTCA | 9.8 |
| E159Q | TTTA | 9.44 |
| E159Q | TTTG | 9.27 |
| E159R | GTTC | 14.77 |
| E159R | GTTG | 13.13 |
| E159R | TCCG | 12.2 |
| E159R | TTGC | 11.82 |
| E159R | TTCG | 11.71 |
| E159R | CCTC | 11.67 |
| E159R | ATTG | 11.35 |
| E159R | CCCC | 11.09 |
| E159R | TTAC | 11.05 |
| E159R | CTTC | 10.88 |
| E159R | TCTG | 10.85 |
| E159R | ATTC | 10.72 |
| E159R | TTCT | 10.71 |
| E159R | TCTC | 10.63 |
| E159R | GTTA | 10.62 |
| E159R | TTCA | 10.59 |
| E159R | CTTG | 10.51 |
| E159R | TCGC | 10.29 |
| E159R | TCTA | 10.16 |
| E159R | ATTA | 10.06 |
| E159R | ACCC | 9.76 |
| E159R | TTTG | 9.55 |
| E159R | CCTA | 9.52 |
| E159R | CTTA | 9.47 |
| E159R | GCGC | 9.32 |
| E159R | ACTC | 8.95 |
| E159R | CCGC | 8.91 |
| E159Y | TTTT | 11.72 |
| E159Y | CTTC | 11.71 |
| E159Y | TTTA | 11.42 |
| E159Y | CTTA | 10.79 |
| E159Y | TTTG | 10.39 |
| E159Y | TTGC | 10.37 |
| E159Y | TTCC | 9.94 |
| E159Y | CTTG | 9.51 |
| E159Y | TCCC | 9.43 |
| G532A | TTTT | 13.95 |
| G532A | CTTG | 12.83 |
| G532A | ATTC | 12.56 |
| G532A | CTTC | 11.75 |
| G532A | TCTC | 11.56 |
| G532A | TTCC | 11.55 |
| G532A | TTCG | 11.5 |
| G532A | TCTA | 11.01 |
| G532A | TTCA | 10.91 |
| G532A | CCCC | 10.36 |
| G532A | CTTA | 10.35 |
| G532A | TTGC | 10.19 |
| G532A | GTTG | 10.18 |
| G532A | TCCC | 10.1 |
| G532A | TCTG | 9.94 |
| G532A | ATTG | 9.6 |
| G532A | TTTA | 9.47 |
| G532A | GTTA | 9.4 |
| G532C | TTTT | 15.09 |
| G532C | TTCC | 12.43 |
| G532C | TTTC | 12.3 |
| G532C | TTGC | 11.56 |
| G532C | CTTA | 11.27 |
| G532C | TTAC | 11.02 |
| G532C | TTTG | 10.94 |
| G532C | TTCA | 10.93 |
| G532C | CTTG | 10.62 |
| G532C | TTTA | 10.31 |
| G532C | TCCC | 10.29 |
| G532C | TCTA | 10.26 |
| G532C | TCTG | 10.19 |
| G532C | GTTA | 9.93 |
| G532C | TCTC | 9.91 |
| G532C | CTTC | 9.78 |
| G532C | GTTG | 8.88 |
| G532D | CTTG | 21.38 |
| G532D | CTTA | 15.81 |
| G532D | TTCC | 15.28 |
| G532D | TTTT | 14.57 |
| G532D | CTTC | 13.93 |
| G532D | TTTC | 13.65 |
| G532D | TTTG | 13.59 |
| G532D | TTCG | 13.54 |
| G532D | TTCT | 13.13 |
| G532D | TTCA | 12.84 |
| G532D | CCCC | 12.66 |
| G532D | TTTA | 12.54 |
| G532D | TTGC | 12.49 |
| G532D | GTTG | 11.87 |
| G532D | TCCG | 11.41 |
| G532D | ATTG | 11.27 |
| G532D | ATTA | 11 |
| G532D | TCCC | 10.91 |
| G532D | ATTC | 10.56 |
| G532D | TCTA | 10.36 |
| G532D | CTCC | 9.98 |
| G532D | TTAC | 9.81 |
| G532D | TCGC | 9.2 |
| G532F | CTTA | 13.56 |
| G532F | ATTG | 11.28 |
| G532F | CTTC | 11.1 |
| G532F | TTCA | 10.82 |
| G532F | TTGC | 10.74 |
| G532F | TTTT | 10.6 |
| G532F | TCTA | 10.52 |
| G532F | TTAC | 10.48 |
| G532F | TTTA | 10.31 |
| G532F | TTTG | 10.25 |
| G532F | ATTA | 10.2 |
| G532F | GTTA | 9.82 |
| G532F | TTTC | 9.64 |
| G532F | ATTC | 9.47 |
| G532F | TCCC | 9.44 |
| G532F | CTTG | 9.28 |
| G532F | TCTC | 9.23 |
| G532F | TCCG | 8.95 |
| G532F | TCTG | 8.91 |
| G532F | CCCC | 8.33 |
| G532F | GTTG | 8.17 |
| G532F | CCTA | 8.08 |
| G532H | TTCA | 16.2 |
| G532H | CTTC | 15.09 |
| G532H | GTTG | 14.43 |
| G532H | TTTT | 13.93 |
| G532H | TTCG | 13.87 |
| G532H | CTTG | 13.72 |
| G532H | TCTG | 13.22 |
| G532H | GTTC | 12.71 |
| G532H | TTCC | 12.66 |
| G532H | CTTA | 12.54 |
| G532H | TTGC | 12.31 |
| G532H | TCTA | 12.2 |
| G532H | TTCT | 12.15 |
| G532H | ATTC | 11.94 |
| G532H | CCCC | 11.91 |
| G532H | TTTC | 11.89 |
| G532H | TCCG | 11.71 |
| G532H | TTTA | 11.67 |
| G532H | TCCC | 11.6 |
| G532H | TTTG | 11.23 |
| G532H | CTCA | 11.23 |
| G532H | ATTA | 11.16 |
| G532H | TCCA | 11.11 |
| G532H | ATTG | 11.02 |
| G532H | TTAC | 10.74 |
| G532H | TCTC | 10.55 |
| G532H | TCGC | 10.42 |
| G532H | CCTA | 10.3 |
| G532H | CTCC | 9.99 |
| G532H | GTTA | 9.77 |
| G532H | CCCA | 9.61 |
| G532H | CCTG | 9.15 |
| G532H | CCTC | 9.03 |
| G532H | CCCG | 8.84 |
| G532H | TCAC | 7.76 |
| G532K | CTCA | 13.19 |
| G532K | GCCC | 12.17 |
| G532K | CTTA | 11.31 |
| G532K | CTTC | 11.17 |
| G532K | TTGC | 10.91 |
| G532K | CTTG | 10.78 |
| G532K | ATTA | 10.64 |
| G532K | GCCG | 10.53 |
| G532K | TTAC | 10.41 |
| G532K | TTTT | 9.96 |
| G532K | TCCA | 9.94 |
| G532K | ACCG | 9.8 |
| G532K | CCCC | 9.69 |
| G532L | CTTC | 15.2 |
| G532L | TTTT | 14.33 |
| G532L | TTTA | 12.17 |
| G532L | CTTG | 12.04 |
| G532L | CTTA | 11.73 |
| G532L | TTGC | 11.67 |
| G532L | TTCA | 11.66 |
| G532L | TTAC | 11.57 |
| G532L | TTTG | 11.5 |
| G532L | ATTA | 11.36 |
| G532L | TTTC | 11.15 |
| G532L | TTCC | 10.89 |
| G532L | ATTG | 10.02 |
| G532L | GTTG | 9.71 |
| G532L | TCTA | 9.46 |
| G532L | CCTA | 9.29 |
| G532L | ATTC | 8.98 |
| G532L | TCCG | 8.77 |
| G532L | TTGA | 8.33 |
| G532N | CTTG | 15.59 |
| G532N | TCCC | 15.09 |
| G532N | TTCG | 14.11 |
| G532N | ATTG | 13.73 |
| G532N | CTTC | 12.97 |
| G532N | TTGC | 12.7 |
| G532N | TTAC | 12.61 |
| G532N | TTCT | 12.13 |
| G532N | ATTA | 12.03 |
| G532N | TCTA | 12.02 |
| G532N | TTCC | 12 |
| G532N | TTTT | 11.95 |
| G532N | TCTC | 11.91 |
| G532N | ATTC | 11.89 |
| G532N | CTTA | 11.8 |
| G532N | TTTC | 11.52 |
| G532N | TTCA | 11.33 |
| G532N | TCTG | 11.3 |
| G532N | TTTA | 11.23 |
| G532N | TTTG | 11.18 |
| G532N | TCCG | 11.17 |
| G532N | GTTG | 11.14 |
| G532N | GTTA | 10.53 |
| G532N | CCCG | 10.51 |
| G532N | GTTC | 9.62 |
| G532N | CTCC | 9.48 |
| G532N | CCCC | 9.46 |
| G532N | TCCA | 9.45 |
| G532N | CCTA | 9.35 |
| G532N | CCTG | 9.28 |
| G532Q | TTTG | 16.46 |
| G532Q | TTGC | 14.95 |
| G532Q | TTCA | 14.64 |
| G532Q | CTTC | 13 |
| G532Q | TTTT | 12.79 |
| G532Q | ATTA | 12.41 |
| G532Q | TTCC | 12.4 |
| G532Q | TTAC | 11.52 |
| G532Q | TTCT | 11.34 |
| G532Q | CTTG | 11.2 |
| G532Q | TCTA | 11.19 |
| G532Q | TTTA | 10.72 |
| G532Q | CTTA | 10.58 |
| G532Q | ATTG | 10.43 |
| G532Q | TTTC | 10.32 |
| G532Q | TTCG | 10.23 |
| G532Q | TCCC | 10.05 |
| G532Q | GTTG | 9.8 |
| G532Q | CCCC | 9.74 |
| G532Q | TCTC | 9.47 |
| G532S | TTAC | 13.85 |
| G532S | TTTT | 13.71 |
| G532S | GTTA | 13.01 |
| G532S | TCTC | 12.51 |
| G532S | TTCA | 12.5 |
| G532S | CTTA | 12.33 |
| G532S | TCCC | 12.3 |
| G532S | ATTG | 12.3 |
| G532S | CTTC | 12.28 |
| G532S | ATTC | 12.08 |
| G532S | TTCC | 11.95 |
| G532S | TCTA | 11.79 |
| G532S | TCTG | 11.78 |
| G532S | TCCG | 11.58 |
| G532S | ATTA | 11.51 |
| G532S | TTTC | 11.49 |
| G532S | CTTG | 11.43 |
| G532S | GTTC | 11.1 |
| G532S | TTCG | 11.03 |
| G532S | GTTG | 10.72 |
| G532S | TTTG | 10.63 |
| G532S | TTTA | 10.56 |
| G532S | TTCT | 10.36 |
| G532S | TTGC | 10.15 |
| G532S | TCGC | 9.46 |
| G532S | TTAG | 9.38 |
| G532S | CCCC | 9.11 |
| G532S | CTCC | 8.12 |
| K116N | CTTA | 12.52 |
| K116N | CTTC | 11.57 |
| K116N | TCCC | 11.05 |
| K116N | TTAC | 10.99 |
| K116N | TCTA | 10.97 |
| K116N | TCTC | 10.9 |
| K116N | TCCA | 10.84 |
| K116N | TCTG | 10.77 |
| K116N | TCCG | 10.73 |
| K116N | TTCG | 10.7 |
| K116N | TTCA | 10.66 |
| K116N | ATTA | 10.47 |
| K116N | TTTT | 10.21 |
| K116N | TTGC | 10.17 |
| K116N | TTTC | 9.92 |
| K116N | CTTG | 9.87 |
| K116N | TTCC | 9.85 |
| K116N | TTCT | 9.64 |
| K116N | CCTA | 9.42 |
| K116N | GTTA | 9.31 |
| K116N | CCTG | 9.21 |
| K116N | CCCC | 9.13 |
| K116N | TCGC | 9.04 |
| K116N | GTTC | 8.91 |
| K116N | CTCA | 8.61 |
| K116N | CCGC | 8.39 |
| K116N | TTGA | 7.69 |
| K116R | TCCG | 13.47 |
| K116R | ATTC | 13.02 |
| K116R | TTAC | 12.37 |
| K116R | GTTC | 11.83 |
| K116R | CCTA | 11.44 |
| K116R | TTCT | 11.35 |
| K116R | ATTG | 11.11 |
| K116R | TCTA | 11.03 |
| K116R | CCCC | 11.03 |
| K116R | CTCC | 10.97 |
| K116R | TCTC | 10.84 |
| K116R | TCGC | 10.29 |
| K116R | GTTG | 10.27 |
| K116R | GCTC | 10.2 |
| K116R | CTTA | 10.15 |
| K116R | TTGC | 10.12 |
| K116R | CCTG | 10.04 |
| K116R | ATTA | 10.02 |
| K116R | TTTT | 9.98 |
| K116R | CCTC | 9.78 |
| K116R | TCCC | 9.72 |
| K116R | GTTA | 9.65 |
| K116R | CCCG | 9.62 |
| K116R | ACTA | 9.61 |
| K116R | TTCG | 9.54 |
| K116R | TCCA | 9.36 |
| K116R | GCGC | 9.27 |
| K116R | TTCC | 9.22 |
| K116R | TTCA | 9.14 |
| K116R | GCTA | 9.14 |
| K116R | CCCA | 8.96 |
| K116R | CTTC | 8.95 |
| K116R | TCTG | 8.93 |
| K116R | TTGG | 8.82 |
| K116R | CCGC | 8.81 |
| K116R | GCTG | 8.67 |
| K116R | ACGC | 8.32 |
| K116R | ACTC | 8.29 |
| K116R | CTCA | 8.06 |
| K120H | TCTC | 10.27 |
| K120H | TTCG | 10.07 |
| K120H | TTTT | 9.97 |
| K120H | CTTG | 9.77 |
| K120H | CTTA | 9.69 |
| K120H | TTCC | 9.3 |
| K120H | TTCA | 9.02 |
| K120H | TTTG | 8.5 |
| K120H | TCGC | 8.43 |
| K120H | TTTC | 8.26 |
| K120H | TTTA | 8.02 |
| K120H | TTGC | 7.89 |
| K120H | TCCC | 7.56 |
| K120H | CCCC | 7.55 |
| K120N | TTTA | 12.31 |
| K120N | TTTT | 11.86 |
| K120N | CCCC | 11.41 |
| K120N | TCTC | 11.4 |
| K120N | TTCC | 11.21 |
| K120N | TCCC | 11.18 |
| K120N | CTTA | 10.93 |
| K120N | TTTC | 10.91 |
| K120N | TTGC | 10.55 |
| K120N | TCTG | 10.41 |
| K120N | ATTA | 10.4 |
| K120N | CTTC | 10.32 |
| K120N | ATTC | 10.31 |
| K120N | TTTG | 9.99 |
| K120N | TTCA | 9.95 |
| K120N | TCTA | 9.86 |
| K120N | CCCG | 9.64 |
| K120N | CTTG | 9.6 |
| K120N | TCCG | 9.49 |
| K120N | TTCG | 9.48 |
| K120N | TTCT | 9.32 |
| K120N | GCGC | 9.07 |
| K120N | CCTA | 8.43 |
| K120N | CTCC | 8.18 |
| K120Q | TTTC | 12.01 |
| K120Q | TTCC | 11.56 |
| K120Q | TTCA | 11.1 |
| K120Q | CTTG | 10.57 |
| K120Q | TTGC | 10.37 |
| K120Q | TTTG | 10.25 |
| K120Q | TTCG | 10.25 |
| K120Q | TTTT | 9.91 |
| K120Q | CTTC | 9.83 |
| K120Q | TTTA | 9.72 |
| K1200 | ATTG | 9.25 |
| K120Q | TTAC | 9.25 |
| K120Q | TCTA | 9.18 |
| K120Q | GTTC | 9.03 |
| K120Q | CTTA | 8.96 |
| K120Q | ATTC | 8.71 |
| K120R | TTCA | 10.74 |
| K120R | CTTA | 9.7 |
| K120T | TTTT | 7.73 |
| K120T | TTCG | 7.73 |
| K120T | TTGA | 7.34 |
| K121D | TTTA | 12.69 |
| K121D | TTTG | 11.07 |
| K121D | TTTC | 10.18 |
| K121G | TTTT | 16.86 |
| K121G | TTCA | 15.35 |
| K121G | TTCC | 15.22 |
| K121G | CTTG | 14.67 |
| K121G | TTGC | 14.52 |
| K121G | CTTA | 14.02 |
| K121G | TTTC | 13.96 |
| K121G | TTTA | 13.42 |
| K121G | CTTC | 13.37 |
| K121G | TTCG | 13.33 |
| K121G | TTTG | 12.87 |
| K121G | TTCT | 12.72 |
| K121G | ATTG | 12.35 |
| K121G | ATTC | 11.96 |
| K121G | ATTA | 11.89 |
| K121G | TTAC | 11.65 |
| K121G | TCCC | 11.12 |
| K121G | TCTA | 10.53 |
| K121G | TCGC | 9.78 |
| K121G | GTTA | 9.62 |
| K121G | GTTG | 9.32 |
| K121G | TCTC | 9.08 |
| K121G | CCCC | 8.06 |
| K121G | GTTC | 7.89 |
| K121H | TTCA | 10.11 |
| K121H | TTGC | 9.93 |
| K121H | TCTA | 9.6 |
| K121H | TTCC | 9.53 |
| K121H | CTTG | 9.11 |
| K121H | TTTT | 8.99 |
| K121H | TTCG | 8.82 |
| K121H | TTTC | 8.53 |
| K121H | TTTA | 8.28 |
| K121H | CTTC | 7.95 |
| K121H | TCCG | 7.77 |
| K121H | CCTG | 7.66 |
| K121H | TCTC | 7.51 |
| K121Q | TTTA | 8.76 |
| K121Q | TTTG | 8.76 |
| K121Q | TTTT | 8.28 |
| K121Q | TTCT | 7.93 |
| K121Q | TTCC | 7.89 |
| K121R | TTCT | 14.44 |
| K121R | CTTC | 13.38 |
| K121R | TTCA | 13.05 |
| K121R | TTTT | 13.03 |
| K121R | CCTG | 12.96 |
| K121R | ATTG | 12.3 |
| K121R | TTAC | 12.28 |
| K121R | CCCC | 12.28 |
| K121R | TCGC | 12.01 |
| K121R | ATTA | 12 |
| K121R | TCCG | 11.99 |
| K121R | TCTA | 11.97 |
| K121R | TTCG | 11.79 |
| K121R | TCTC | 11.72 |
| K121R | TTGC | 11.55 |
| K121R | CCGC | 11.31 |
| K121R | GTTA | 11.23 |
| K121R | GTTG | 11.05 |
| K121R | TTTG | 10.86 |
| K121R | ATTC | 10.84 |
| K121R | CTTA | 10.83 |
| K121R | CCTA | 10.71 |
| K121R | CCTC | 10.52 |
| K121R | TTCC | 10.4 |
| K121R | CCCG | 10.37 |
| K121R | CTCC | 10.21 |
| K121R | TCCA | 10.18 |
| K121R | TTTA | 10.07 |
| K121R | TCCC | 9.9 |
| K121R | CTTG | 9.72 |
| K121R | GCGC | 9.67 |
| K121R | TCAC | 9.59 |
| K121R | CCCA | 9.45 |
| K121R | GTTC | 9.23 |
| K121R | CTCA | 9.04 |
| K121R | TTTC | 8.93 |
| K121R | TCTG | 8.87 |
| K121S | TTGC | 11.77 |
| K121S | CCTG | 11.74 |
| K121S | TCCA | 11.66 |
| K121S | TTTT | 10.88 |
| K121S | TTCA | 10.85 |
| K121S | CCTC | 10.72 |
| K121S | TCTC | 10.62 |
| K121S | CTTA | 10.33 |
| K121S | TTCT | 10.1 |
| K121S | TCTA | 10.06 |
| K121S | CCCC | 10.02 |
| K121S | TTCC | 9.9 |
| K121S | CTTG | 9.88 |
| K121S | TCCC | 9.81 |
| K121S | TTCG | 9.71 |
| K121S | CTCC | 9.65 |
| K121S | TCCG | 9.59 |
| K121S | CTTC | 9.59 |
| K121S | ATTC | 9.53 |
| K121S | TCTG | 9.47 |
| K121S | TTAC | 9.46 |
| K121S | TTTA | 9.31 |
| K121S | GCGC | 9.26 |
| K121S | GTTC | 9.25 |
| K121S | ATTA | 9.2 |
| K121S | CCGC | 9.14 |
| K121S | ACTA | 8.94 |
| K121S | GCCC | 8.9 |
| K121S | TTTC | 8.89 |
| K121S | CTTT | 8.79 |
| K121S | TTGA | 8.76 |
| K121S | TCGC | 8.63 |
| K121S | CCCG | 8.61 |
| K121S | GTTA | 8.5 |
| K121S | GTTG | 8.35 |
| K121S | ACTG | 7.89 |
| K121S | CTCG | 7.8 |
| K121S | GCTA | 7.57 |
| K121T | TTTC | 13.85 |
| K121T | TTTT | 13.26 |
| K121T | TTTA | 13.02 |
| K121T | TTTG | 12.09 |
| K121T | CTTA | 11.65 |
| K121T | CTTC | 10.99 |
| K121T | CTTG | 10.52 |
| K121T | TTCA | 10.44 |
| K121T | TTCC | 10.36 |
| K121T | TTGC | 9.85 |
| K538C | CTTC | 9.67 |
| K538F | TTTA | 17.03 |
| K538F | TTTC | 15.83 |
| K538F | CTTG | 11.01 |
| K538F | CTTC | 10.62 |
| K538F | CTTA | 9.14 |
| K538G | TTTC | 11.42 |
| K538G | TTTA | 10.22 |
| K538G | TTTG | 9.79 |
| K538G | CTTC | 9.18 |
| K538H | TTTG | 11.31 |
| K538H | CTTC | 10.74 |
| K538L | TTTC | 10.95 |
| K538L | TTTA | 10 |
| K538L | CTTA | 9.31 |
| K538M | TTTG | 13.69 |
| K538M | TTTA | 13.09 |
| K538M | TTTC | 11.36 |
| K538M | CTTC | 10 |
| K538M | CTTA | 9.97 |
| K538Q | CTTC | 13.23 |
| K538Q | TTTC | 12.93 |
| K538Q | TTTA | 12.91 |
| K538Q | CTTA | 11.77 |
| K538Q | TTTG | 11.17 |
| K538Q | CTTG | 11.03 |
| K538R | TTTG | 12.33 |
| K538R | TTTC | 12.28 |
| K538R | TTTA | 12.26 |
| K538R | CTTA | 11.95 |
| K538R | TTTT | 9.32 |
| K538R | CTTG | 9.16 |
| K538V | CTTC | 18.4 |
| K538V | CTTG | 18.05 |
| K538V | TTTA | 17.53 |
| K538V | TTTG | 17.49 |
| K538V | TTTC | 15.98 |
| K538V | CTTA | 14.13 |
| K538W | TTTC | 11.69 |
| K538W | TTTG | 11.18 |
| K538W | TTTA | 11.03 |
| K538W | TATA | 10.38 |
| K538W | CTTC | 10 |
| K538W | TATC | 9.66 |
| K538W | CTTA | 9.26 |
| K538Y | CTTG | 9.14 |
| K538Y | CTTA | 8.3 |
| K538Y | CTTC | 8.08 |
| K591A | TTTG | 8.62 |
| K591F | TTTA | 13.44 |
| K591F | TTTG | 12.35 |
| K591F | TTTC | 11.17 |
| K591F | TTTT | 10.9 |
| K591G | TTTA | 11.71 |
| K591G | TTTC | 11.25 |
| K591G | TTTT | 10.92 |
| K591G | TTTG | 10.78 |
| K591H | TTTC | 16.42 |
| K591H | TTTA | 14.07 |
| K591H | TTTG | 14.02 |
| K591H | TTTT | 12.41 |
| K591R | TTTG | 10.31 |
| K591R | TTCC | 9.81 |
| K591R | TTTC | 9.07 |
| K591R | CTTG | 8.61 |
| K591S | TTTT | 11.24 |
| K591S | TTCC | 9.81 |
| K591S | TTTA | 9.37 |
| K591S | TTTC | 9.23 |
| K591S | CTTG | 8.98 |
| K591S | TTTG | 8.89 |
| K591S | TTCG | 8.04 |
| K591W | TTTA | 13.93 |
| K591W | TTTC | 11.17 |
| K591W | TTTG | 11.14 |
| K591Y | TTTC | 12.29 |
| K591Y | TTTA | 11.4 |
| K591Y | TTTG | 10.79 |
| K595H | TTGC | 12.45 |
| K595H | TTTT | 11.42 |
| K595H | TTTG | 10.91 |
| K595H | TTCA | 10.39 |
| K595H | TTTC | 10.21 |
| K595H | TTCG | 9.36 |
| K595L | TTTA | 16.84 |
| K595L | TTCC | 15.92 |
| K595L | TTTG | 14.92 |
| K595L | CCTG | 13.79 |
| K595L | TTCG | 13.45 |
| K595L | TTTC | 13.12 |
| K595L | TCTA | 12.6 |
| K595L | TCTC | 12.15 |
| K595L | TCCA | 12.14 |
| K595L | TCTG | 11.7 |
| K595L | ACTC | 11.46 |
| K595L | TCCG | 11.37 |
| K595L | CCTC | 10.6 |
| K595L | TTCA | 10.5 |
| K595L | TCCC | 10.24 |
| K595L | ACTG | 9.69 |
| K595M | TTGC | 13.18 |
| K595M | TTTA | 12.88 |
| K595M | CCTG | 12.36 |
| K595M | TTTG | 12.07 |
| K595M | TCCC | 11.4 |
| K595M | TTTT | 11.26 |
| K595M | CCTC | 10.96 |
| K595M | TCTG | 10.95 |
| K595M | TTTC | 10.84 |
| K595M | TTCA | 10.81 |
| K595M | ACTG | 10.74 |
| K595M | TCTA | 10.34 |
| K595M | TCTT | 10.1 |
| K595M | TCCG | 9.83 |
| K595M | ACGC | 9.6 |
| K595M | TTCC | 9.51 |
| K595M | CCCC | 9.4 |
| K595M | TCGG | 9.21 |
| K595M | GCTG | 9.12 |
| K595M | CTTC | 8.62 |
| K595M | CTTA | 8.14 |
| K595Q | TCCG | 15.13 |
| K595Q | TTCC | 14.56 |
| K595Q | TTCA | 13.57 |
| K595Q | TCTG | 13.57 |
| K595Q | TCTC | 12.93 |
| K595Q | TTTG | 12.45 |
| K595Q | TTTA | 12.22 |
| K595Q | TCTA | 12.06 |
| K595Q | TTCG | 11.85 |
| K595Q | TTTC | 11.69 |
| K595Q | TCCC | 10.62 |
| K595Q | ACCC | 10.09 |
| K595Q | CCCC | 9.21 |
| K595R | TCTT | 8.5 |
| K595R | TCCC | 8 |
| K595S | TCCC | 14.55 |
| K595S | TTGC | 14.34 |
| K595S | TCGC | 14.23 |
| K595S | TTTA | 13.22 |
| K595S | TCTC | 12.48 |
| K595S | TCTA | 12.47 |
| K595S | TCCG | 12.3 |
| K595S | CCGC | 12.29 |
| K595S | ACGC | 11.62 |
| K595S | TTCC | 11.53 |
| K595S | TTTC | 10.96 |
| K595S | TCGG | 10.45 |
| K595S | CCCC | 10.13 |
| K595S | TTCA | 9.48 |
| K595S | TTCG | 8.92 |
| K595W | CCCC | 11.92 |
| K595W | TCGA | 11.62 |
| K595W | ACGG | 11.48 |
| K595W | ACCC | 11.35 |
| K595W | CCTG | 11.35 |
| K595W | TCTC | 11.28 |
| K595W | TTTT | 10.53 |
| K595W | GCCG | 10.42 |
| K595W | TTTA | 10.21 |
| K595W | ACTA | 10.18 |
| K595W | ACGA | 9.87 |
| K595W | GCGC | 9.81 |
| K595W | ATGC | 9.77 |
| K595W | TCGT | 9.71 |
| K595W | ATTG | 9.63 |
| K595W | TCCT | 9.25 |
| K595W | CCGG | 9.19 |
| K595W | TTGT | 9.08 |
| K595W | TCGG | 9.03 |
| K595W | TTCT | 8.95 |
| K595W | ACTG | 8.88 |
| K595Y | ACCC | 18.7 |
| K595Y | TCCC | 18.62 |
| K595Y | ACCG | 18.58 |
| K595Y | CCCG | 18.42 |
| K595Y | TTCG | 17.75 |
| K595Y | GCGC | 17.47 |
| K595Y | TCCA | 16.89 |
| K595Y | CCGC | 16.66 |
| K595Y | CCGG | 16.59 |
| K595Y | GCGG | 16.46 |
| K595Y | TCCG | 16.23 |
| K595Y | CCCC | 16.05 |
| K595Y | TTTC | 15.82 |
| K595Y | TCGA | 14.88 |
| K595Y | TCGG | 14.74 |
| K595Y | TTTA | 14.31 |
| K595Y | CCGA | 14.29 |
| K595Y | TTTG | 14.12 |
| K595Y | CTGC | 14.03 |
| K595Y | TCGT | 14.01 |
| K595Y | ACGC | 13.83 |
| K595Y | ACGA | 13.62 |
| K595Y | ACGG | 13.55 |
| K595Y | TCTG | 13.36 |
| K595Y | TCTC | 13.31 |
| K595Y | TCGC | 13.26 |
| K595Y | GCCG | 13.19 |
| K595Y | GCCC | 13.15 |
| K595Y | TTGA | 12.32 |
| K595Y | TTCT | 12.28 |
| K595Y | TTCA | 12.22 |
| K595Y | TTGT | 12.12 |
| K595Y | TTGG | 12.01 |
| K595Y | TCTA | 11.81 |
| K595Y | GCGA | 11.57 |
| K595Y | CCCA | 11.52 |
| K595Y | CTCG | 11.51 |
| K595Y | TTGC | 11.46 |
| K595Y | TTCC | 11.3 |
| K595Y | ACCA | 11.3 |
| K595Y | ACTG | 11.29 |
| K595Y | CCTG | 10.54 |
| K595Y | CTCC | 10.3 |
| K595Y | ACTC | 9.54 |
| K595Y | ATGC | 9.28 |
| K600G | ATTG | 14.1 |
| K600G | TTTT | 13.53 |
| K600G | TCTC | 13.48 |
| K600G | CTTG | 13.02 |
| K600G | TTTA | 12.84 |
| K600G | CTTC | 12.82 |
| K600G | TCTG | 12.69 |
| K600G | ATTA | 12.52 |
| K600G | TCTA | 12.41 |
| K600G | TTCA | 12.15 |
| K600G | TTTC | 12.11 |
| K600G | CTTA | 12.01 |
| K600G | TTCG | 11.67 |
| K600G | TTCC | 11.66 |
| K600G | TTTG | 11.58 |
| K600G | TTAC | 11.52 |
| K600G | TCCC | 11.4 |
| K600G | CCTG | 11.38 |
| K600G | CCCC | 10 |
| K600G | GTTA | 9.75 |
| K600G | GTTG | 9.72 |
| K600G | TCCG | 9.69 |
| K600G | TTCT | 9.48 |
| K600G | CCTA | 9.4 |
| K600G | TCCA | 9.38 |
| K600H | CTTC | 16.65 |
| K600H | TTTG | 14.45 |
| K600H | TTCC | 14.11 |
| K600H | TTTT | 13.78 |
| K600H | TTCA | 13.27 |
| K600H | CTTA | 12.42 |
| K600H | TCTA | 12.26 |
| K600H | TCTC | 12.2 |
| K600H | TCCC | 12.16 |
| K600H | ATTG | 12.02 |
| K600H | ATTA | 11.77 |
| K600H | CTTG | 11.23 |
| K600H | TTCG | 11.13 |
| K600H | TTTC | 10.82 |
| K600H | TTAC | 10.54 |
| K600H | GTTC | 9.1 |
| K600H | GTTG | 8.65 |
| K600H | CCGC | 8.41 |
| K600R | ATTC | 13.41 |
| K600R | TTGC | 12.52 |
| K600R | TTCT | 11.83 |
| K600R | TTCC | 11.61 |
| K600R | TTCA | 11.08 |
| K600R | TTTT | 11.07 |
| K600R | CCCC | 11.05 |
| K600R | TCGC | 11.02 |
| K600R | TCTA | 10.96 |
| K600R | GTTC | 10.91 |
| K600R | TCCA | 10.84 |
| K600R | TCCC | 10.63 |
| K600R | CCTC | 10.42 |
| K600R | TTCG | 10.1 |
| K600R | CTCA | 8.96 |
| K601H | CCTG | 14.33 |
| K601H | CTTG | 12.41 |
| K601H | TCTA | 12.13 |
| K601H | TCTC | 11.97 |
| K601H | ATTC | 11.89 |
| K601H | TCCG | 11.84 |
| K601H | TTTG | 11.78 |
| K601H | CCCG | 11.7 |
| K601H | TTTT | 11.35 |
| K601H | CCTA | 11.15 |
| K601H | TTGC | 10.89 |
| K601H | CTTC | 10.79 |
| K601H | TTTC | 10.65 |
| K601H | CTTA | 10.45 |
| K601H | CCCC | 10.42 |
| K601H | TTCG | 10.39 |
| K601H | ATTG | 10.26 |
| K601H | TTTA | 10.21 |
| K601H | ATTA | 10.2 |
| K601H | TTCA | 10.12 |
| K601H | TCTG | 10.09 |
| K601H | CCTC | 9.82 |
| K601H | TCGC | 9.4 |
| K601H | GCTC | 8.68 |
| K601Q | CTTA | 13.97 |
| K601Q | TTTG | 11.17 |
| K601Q | TTTC | 10.17 |
| K601Q | CTTG | 9.73 |
| K601Q | TTTT | 9.44 |
| K601R | TCTA | 12.21 |
| K601R | TTGC | 12.09 |
| K601R | ATTC | 11.08 |
| K601R | TTTT | 10.98 |
| K601R | TTCC | 10.95 |
| K601R | TCTG | 10.8 |
| K601R | TTCA | 10.77 |
| K601R | CCTA | 10.4 |
| K601R | ATTG | 10.33 |
| K601R | CTTG | 10.31 |
| K601R | CTTC | 10.11 |
| K601R | TCTC | 9.85 |
| K601R | TCCG | 9.65 |
| K601R | ATTA | 9.5 |
| K601R | TCCA | 8.91 |
| K601R | CCTG | 8.54 |
| K601R | TTTC | 8.41 |
| K601T | CTTA | 12.42 |
| K601T | TTTC | 11.55 |
| K601T | TTCC | 11.36 |
| K601T | TTCG | 11.26 |
| K601T | TTTT | 11.13 |
| K601T | CTTC | 11.06 |
| K601T | TTTA | 10.99 |
| K601T | TTCA | 10.85 |
| K601T | TTTG | 10.2 |
| K601T | TCCC | 10.17 |
| K601T | TCCG | 9.65 |
| K601T | CCTG | 9.53 |
| K601T | TCTC | 9.29 |
| K601T | CCCC | 8.18 |
| L585F | TTTG | 13.66 |
| L585F | TTTT | 11.79 |
| L585F | TTTA | 11.72 |
| L585F | TTTC | 10.51 |
| L585G | TTCC | 13.33 |
| L585G | TTAC | 11.98 |
| L585G | CTTG | 11.26 |
| L585G | TTTT | 11.21 |
| L585G | TCCC | 11.09 |
| L585G | TTCA | 10.97 |
| L585G | CTTC | 10.32 |
| L585G | TTTA | 9.92 |
| L585G | TCTC | 9.8 |
| L585G | TTGC | 9.57 |
| L585G | CTTA | 9.48 |
| L585G | TCAC | 9.48 |
| L585G | TTTG | 9.02 |
| L585G | TTTC | 8.8 |
| L585H | TTCC | 9.49 |
| L585H | TTTT | 8.29 |
| L585H | TCTC | 8.06 |
| M592A | TTTG | 15.81 |
| M592A | TTTA | 11.41 |
| M592A | TTTC | 10.98 |
| M592A | CTTA | 10.8 |
| M592A | TTCA | 10.53 |
| M592A | TCTA | 10.38 |
| M592A | TTTT | 9.48 |
| M592E | TTTA | 12.63 |
| M592E | TTTG | 11.85 |
| M592E | TTTC | 11.64 |
| M592Q | TTCA | 12.54 |
| M592Q | TTTA | 10.68 |
| M592Q | TCCG | 10.66 |
| M592Q | CTTG | 10.19 |
| M592Q | TTCG | 9.93 |
| M592Q | CTTA | 9.45 |
| M592Q | CCCG | 9.41 |
| M592Q | TCTG | 9.39 |
| M592Q | TTTG | 9.37 |
| M592Q | CCCA | 9.22 |
| M592Q | TCCC | 9.17 |
| M592Q | TTCC | 9.16 |
| M592Q | CCCC | 8.93 |
| M592Q | TTTC | 8.89 |
| M592Q | CTCA | 8.41 |
| Q529A | TCTG | 13.89 |
| Q529A | CCCC | 13.38 |
| Q529A | TTCC | 12.5 |
| Q529A | ATTC | 12.31 |
| Q529A | TCCA | 12.26 |
| Q529A | TCTC | 12.21 |
| Q529A | TTGC | 11.88 |
| Q529A | CCCG | 11.81 |
| Q529A | TTTT | 11.69 |
| Q529A | TTCG | 11.43 |
| Q529A | TCTA | 11.31 |
| Q529A | CTTC | 11.2 |
| Q529A | ATTG | 10.94 |
| Q529A | CCTA | 10.85 |
| Q529A | ATTA | 10.79 |
| Q529A | TTTA | 10.73 |
| Q529A | CTTA | 10.63 |
| Q529A | CCTG | 10.54 |
| Q529A | CTTG | 10.47 |
| Q529A | TCCG | 10.4 |
| Q529A | CCTC | 10.38 |
| Q529A | TCCC | 10.18 |
| Q529A | TTCA | 10.16 |
| Q529A | TTCT | 9.89 |
| Q529A | CCGC | 9.68 |
| Q529A | TTTG | 9.62 |
| Q529A | TTTC | 9.51 |
| Q529A | GTTA | 9.4 |
| Q529D | TTTC | 13.64 |
| Q529D | TTTT | 13.03 |
| Q529D | TTTG | 12.45 |
| Q529D | TTCC | 12.36 |
| Q529D | CTTC | 12.27 |
| Q529D | TTTA | 12.24 |
| Q529D | TTGC | 10.98 |
| Q529D | CTTG | 10.85 |
| Q529D | CTTA | 10.81 |
| Q529D | TTCA | 10.36 |
| Q529D | TCCC | 8.52 |
| Q529F | CTTA | 14.01 |
| Q529F | CTTG | 13.75 |
| Q529F | TTTG | 12.84 |
| Q529F | TTTA | 11.62 |
| Q529F | TTTC | 10.91 |
| Q529G | CTTC | 13.9 |
| Q529G | TTCA | 11.91 |
| Q529G | TCTA | 11.71 |
| Q529G | CCCC | 11.18 |
| Q529G | TTTA | 10.86 |
| Q529G | TCTG | 10.79 |
| Q529G | TTTC | 10.63 |
| Q529G | CTTA | 10.61 |
| Q529G | TTTG | 10.19 |
| Q529G | CTTG | 9.88 |
| Q529G | CCTA | 9.67 |
| Q529G | TCCC | 9.5 |
| Q529G | TTCG | 9.15 |
| Q529G | TCTC | 9.11 |
| Q529G | ATTC | 8.98 |
| Q529G | CCTG | 8.96 |
| Q529G | ATTA | 8.33 |
| Q529G | TCGC | 8.2 |
| Q529G | TCCG | 8.11 |
| Q529H | TTTC | 11.09 |
| Q529H | CTTG | 10.43 |
| Q529H | TTTA | 10.39 |
| Q529H | TTCC | 10.29 |
| Q529H | TTCG | 10.29 |
| Q529H | CTTC | 9.59 |
| Q529H | ATTG | 9.52 |
| Q529H | TTTG | 9.12 |
| Q529H | TTCA | 9.07 |
| Q529H | ATTA | 8.47 |
| Q529N | TCGC | 13.73 |
| Q529N | TCTC | 13.48 |
| Q529N | TCCG | 12.62 |
| Q529N | TTTT | 12.48 |
| Q529N | TCTG | 12.38 |
| Q529N | TTGC | 12.3 |
| Q529N | TTCC | 12.2 |
| Q529N | TCCA | 11.98 |
| Q529N | TCCC | 11.38 |
| Q529N | TCTA | 11.04 |
| Q529N | TTCA | 10.89 |
| Q529N | TTCG | 10.81 |
| Q529N | ATTC | 10.79 |
| Q529N | CCCC | 10.6 |
| Q529N | TTTA | 10.57 |
| Q529N | CTTG | 10.24 |
| Q529N | CTTA | 10.18 |
| Q529N | TTCT | 9.91 |
| Q529N | CTTC | 9.87 |
| Q529N | ATTA | 9.85 |
| Q529N | TTTC | 9.83 |
| Q529N | CCTA | 9.49 |
| Q529N | GTTA | 9.32 |
| Q529N | GTTC | 9.18 |
| Q529N | TCTT | 8.71 |
| Q529N | TCAC | 8.3 |
| Q529N | CCCG | 8.23 |
| Q529P | TTTA | 14.13 |
| Q529P | TTTG | 12.06 |
| Q529P | TTTC | 10.87 |
| Q529S | TTCA | 11.88 |
| Q529S | CTTA | 11.14 |
| Q529S | TTTG | 11.09 |
| Q529S | TTTA | 11.06 |
| Q529S | TCTA | 10.17 |
| Q529S | TTTC | 10.1 |
| Q529S | CCCC | 10.1 |
| Q529S | TTTT | 10.03 |
| Q529S | TTCC | 10.02 |
| Q529S | CTTG | 9.9 |
| Q529S | TCCC | 9.77 |
| Q529S | TCTG | 9.68 |
| Q529S | ATTA | 8.81 |
| Q529T | CTTG | 15.39 |
| Q529T | TTCA | 14.62 |
| Q529T | TCTG | 13.74 |
| Q529T | TCTC | 13.63 |
| Q529T | TCTA | 13.2 |
| Q529T | TTCC | 13.17 |
| Q529T | TTTT | 12.6 |
| 2529T | TCCG | 12.44 |
| Q529T | TCCA | 12.34 |
| Q529T | ATTA | 12.15 |
| Q529T | CTTC | 12.13 |
| Q529T | TTCG | 11.89 |
| Q529T | TCCC | 11.35 |
| Q529T | TTGC | 11.17 |
| Q529T | CCTA | 11.07 |
| Q529T | GTTG | 10.96 |
| Q529T | ATTG | 10.9 |
| Q529T | TTTC | 10.88 |
| Q529T | TTTA | 10.73 |
| Q529T | TTTG | 10.51 |
| Q529T | ATTC | 10.41 |
| Q529T | CTTA | 10.37 |
| Q529T | CCTC | 10.12 |
| Q529T | TCGC | 10.06 |
| Q529T | GTTA | 9.83 |
| Q529T | CCCC | 9.78 |
| Q529T | GTTC | 9.72 |
| Q529T | CCTG | 9.3 |
| Q529T | TTCT | 8.86 |
| Q529T | TTAC | 7.99 |
| Q529W | TTTG | 15.75 |
| Q529W | CTTA | 14.5 |
| Q529W | TTTA | 12.69 |
| Q529W | TTTC | 11.31 |
| Q529W | CTTC | 9.28 |
| S599G | CTTG | 12.09 |
| S599G | TTTT | 11.66 |
| S599G | TTTA | 10.2 |
| S599G | TTCC | 9.87 |
| S599G | CCCC | 9.47 |
| S599G | TTCA | 9.36 |
| S599H | TTTA | 10.82 |
| S599H | TTTG | 9.61 |
| S599N | CTTA | 13.93 |
| S599N | TTTG | 13.04 |
| S599N | TTTA | 11.64 |
| S599N | TTTC | 11.3 |
| S599N | CTTC | 11.15 |
| S599N | CTTG | 10.54 |
| S599N | TTCA | 8.73 |
| T148A | TTTC | 13.06 |
| T148A | TTTA | 11.98 |
| T148A | TTTT | 11.53 |
| T148A | TTGC | 11.48 |
| T148A | TTTG | 11.33 |
| T148A | TTCC | 10.97 |
| T148A | CTTG | 9.83 |
| T148A | TTCA | 9.26 |
| T148C | TTCA | 15.44 |
| T148C | TTTG | 15.17 |
| T148C | TTTC | 15.09 |
| T148C | CTTG | 14.75 |
| T148C | CTTA | 13.98 |
| T148C | CTTC | 13.67 |
| T148C | TTTT | 13.43 |
| T148C | TTTA | 13.16 |
| T148C | TTCC | 12.16 |
| T148C | TTGC | 11.1 |
| T148C | TCTA | 10.77 |
| T148C | GTTG | 10.01 |
| T148C | TCTC | 9.93 |
| T148H | TTTC | 14.35 |
| T148H | TTTG | 13.68 |
| T148H | TTTA | 13.64 |
| T148S | TTTT | 13.35 |
| T148S | CTTA | 12.93 |
| T148S | TTTA | 12.48 |
| T148S | TTCG | 12.31 |
| T148S | TTCA | 11.85 |
| T148S | TTGC | 11.56 |
| T148S | TTTG | 11.55 |
| T148S | TTCC | 11.39 |
| T148S | TTTC | 11.01 |
| T148S | CTTC | 11.01 |
| T148S | CTTG | 10.8 |
| T148S | ATTA | 9.58 |
| T148S | TCTA | 8.54 |
| T148S | ATTG | 8.31 |
| T149C | TTTC | 11.35 |
| T149C | TTTA | 11.08 |
| T149C | TTTG | 10.49 |
| T149C | CTTA | 10.35 |
| T149C | TTTT | 9.98 |
| T149F | TTTC | 13.56 |
| T149F | TTTG | 13.11 |
| T149F | TTTA | 11.72 |
| T149G | TTTG | 12.06 |
| T149G | TTTA | 10.59 |
| T149G | CTTG | 10.14 |
| T149G | TCCC | 10.11 |
| T149G | GTTG | 10.08 |
| T149G | TTTC | 9.96 |
| T149G | TTCA | 9.78 |
| T149G | TTTT | 9.77 |
| T149G | CCCC | 9.29 |
| T149G | ATTG | 9.26 |
| T149G | TTCC | 9.2 |
| T149G | CTTC | 8.83 |
| T149G | TCTA | 8.83 |
| T149G | ATTC | 8.6 |
| T149G | TCCG | 8.58 |
| T149G | CCCG | 8.57 |
| T149G | TCTC | 8.45 |
| T149H | TTTA | 15.77 |
| T149H | TTTC | 11.31 |
| T149N | TTTA | 13.97 |
| T149N | TTTC | 13.27 |
| T149N | TTTG | 10.86 |
| T149P | TTTC | 10.27 |
| T149P | TTTA | 10.14 |
| T149P | TTTG | 9.22 |
| T149S | TTCC | 14.11 |
| T149S | CTTC | 14.01 |
| T149S | ATTG | 13.88 |
| T149S | TCTG | 13.24 |
| T149S | TTCA | 12.74 |
| T149S | ATTC | 12.36 |
| T149S | CTTA | 12.32 |
| T149S | TTTT | 12.24 |
| T149S | CTTG | 11.87 |
| T149S | CCTG | 11.55 |
| T149S | ATTA | 11.51 |
| T149S | TCTC | 11.38 |
| T149S | TTCG | 11.34 |
| T149S | TCTA | 11.17 |
| T149S | GTTA | 10.66 |
| T149S | GTTC | 10.45 |
| T149S | GTTG | 10.44 |
| T149S | TCGC | 10.43 |
| T149S | CCCC | 10.4 |
| T149S | TCCG | 10.35 |
| T149S | TTGC | 10.2 |
| T149S | TCCA | 9.82 |
| T149S | CCTA | 9.7 |
| T149S | TTTC | 9.68 |
| T149S | TCCC | 9.33 |
| T149S | TTCT | 9.18 |
| T149S | TTTA | 9.08 |
| T149S | CCGC | 8.88 |
| T149S | CCCG | 8.63 |
| T149S | CCTC | 8.45 |
| T149V | TTCA | 13.47 |
| T149V | TTTA | 13.34 |
| T149V | TTCC | 12.51 |
| T149V | TTTG | 12.3 |
| T149V | TTTT | 11.86 |
| T149V | TTCG | 11.21 |
| T149V | TTTC | 10.58 |
| T149V | TCCC | 9.41 |
| T149V | CTTG | 8.81 |
| T149V | TCTC | 8.75 |
| T152E | CCTC | 18.07 |
| T152E | CTTC | 16.54 |
| T152E | TTGC | 15.69 |
| T152E | TTCA | 14.71 |
| T152E | TCTG | 14.5 |
| T152E | TCCC | 14.43 |
| T152E | TCGC | 14.25 |
| T152E | TCTC | 14.21 |
| T152E | GTTC | 14.11 |
| T152E | TTTC | 14.1 |
| T152E | TTTT | 14.1 |
| T152E | ATTC | 13.83 |
| T152E | CTTG | 13.72 |
| T152E | TTTA | 13.71 |
| T152E | TCCG | 13.52 |
| T152E | CCCC | 12.98 |
| T152E | TTCC | 12.87 |
| T152E | TTCG | 12.63 |
| T152E | ACTC | 12.45 |
| T152E | CCGC | 12.05 |
| T152E | GCGC | 12.01 |
| T152E | CTTA | 11.75 |
| T152E | TTTG | 11.67 |
| T152E | TTCT | 11.58 |
| T152E | TCAC | 10.76 |
| T152E | CCAC | 10.72 |
| T152E | GTTG | 10.4 |
| T152F | TTCC | 16.18 |
| T152F | CTTC | 15.86 |
| T152F | CTTA | 15.51 |
| T152F | TTTC | 14.6 |
| T152F | TTGC | 14.54 |
| T152F | TTCA | 14.36 |
| T152F | TTTT | 13.43 |
| T152F | TTTG | 13.17 |
| T152F | CTTG | 12.61 |
| T152F | ATTA | 12.5 |
| T152F | TCTC | 11.97 |
| T152F | TTCG | 11.93 |
| T152F | TCCC | 11.76 |
| T152F | TTTA | 11.14 |
| T152F | TCGC | 10.44 |
| T152F | CCCC | 9.98 |
| T152H | TCCC | 12.04 |
| T152H | TTGC | 11.41 |
| T152H | CCCC | 11.3 |
| T152H | TTTT | 11.27 |
| T152H | CTTA | 11.1 |
| T152H | TTCC | 11.06 |
| T152H | TCTC | 10.51 |
| T152H | CTTG | 10.27 |
| T152H | TTTC | 10.23 |
| T152H | TTTA | 10.06 |
| T152H | TCGC | 10.02 |
| T152H | ATTC | 10.01 |
| T152H | TTTG | 9.94 |
| T152H | TCTG | 9.88 |
| T152H | CCTC | 9.85 |
| T152H | CTTC | 9.81 |
| T152H | TCCG | 9.76 |
| T152H | TTCA | 9.71 |
| T152H | ATTA | 9.64 |
| T152H | TCTA | 9.63 |
| T152H | TTCT | 9.34 |
| T152H | TCCA | 9.05 |
| T152H | TTAC | 8.95 |
| T152H | ATTG | 8.74 |
| T152H | TTCG | 8.46 |
| T152K | CTTG | 14.08 |
| T152K | CCGC | 13.88 |
| T152K | CCTA | 13.58 |
| T152K | ATTA | 13.28 |
| T152K | ACTC | 13.28 |
| T152K | TCCC | 13.26 |
| T152K | GTTG | 13.22 |
| T152K | GCTC | 13.21 |
| T152K | CCCC | 12.99 |
| T152K | TTCT | 12.84 |
| T152K | TCAC | 12.63 |
| T152K | GCTA | 12.51 |
| T152K | CCTG | 12.5 |
| T152K | CTTA | 12.46 |
| T152K | GTTC | 12.46 |
| T152K | ATTG | 12.29 |
| T152K | ATTC | 12.24 |
| T152K | TCTC | 12.23 |
| T152K | TCTG | 12.2 |
| T152K | CTTC | 12.12 |
| T152K | TTCA | 11.99 |
| T152K | TTCG | 11.88 |
| T152K | CCTC | 11.81 |
| T152K | CCAC | 11.76 |
| T152K | ACTG | 11.5 |
| T152K | TCTA | 11.44 |
| T152K | GCGC | 11.41 |
| T152K | TCGC | 11.15 |
| T152K | TCCA | 11.08 |
| T152K | GTTA | 10.94 |
| T152K | TTAC | 10.94 |
| T152K | CTCC | 10.78 |
| T152K | TTTA | 10.44 |
| T152K | TCGA | 10.16 |
| T152K | ACTA | 10.14 |
| T152K | TTCC | 10.09 |
| T152K | GCCC | 9.97 |
| T152K | TTTT | 9.85 |
| T152K | ACGC | 9.71 |
| T152K | TTGC | 9.68 |
| T152K | GCTG | 9.59 |
| T152K | TCCG | 9.4 |
| T152K | TTGA | 9.39 |
| T152K | CCCG | 9.34 |
| T152K | ACCC | 9.3 |
| T152K | TTTG | 9.21 |
| T152K | CTTT | 8.75 |
| T152L | CTTA | 15.37 |
| T152L | ATTC | 14.54 |
| T152L | TCGC | 13.95 |
| T152L | TCTA | 13.76 |
| T152L | CTTG | 13.75 |
| T152L | CTTC | 13.54 |
| T152L | TCTC | 13.15 |
| T152L | CCCC | 12.44 |
| T152L | TTGC | 12.23 |
| T152L | TTCC | 12.04 |
| T152L | TTCG | 11.39 |
| T152L | ATTG | 10.85 |
| T152L | TTTT | 10.5 |
| T152L | CCTA | 10.43 |
| T152L | TTAC | 10.32 |
| T152L | GTTG | 9.55 |
| T152L | CCTC | 9.38 |
| T152L | TCTG | 9.22 |
| T152L | TTTA | 8.96 |
| T152L | CCTG | 8.96 |
| T152Q | TCGC | 11.48 |
| T152Q | CCTG | 11.36 |
| T152Q | CCTA | 11.27 |
| T152Q | CCCA | 10.85 |
| T152Q | TCAC | 9.99 |
| T152Q | CCGC | 9.9 |
| T152Q | ATTC | 9.85 |
| T152Q | TCTG | 9.82 |
| T152Q | CTTC | 9.78 |
| T152Q | TTGC | 9.7 |
| T152Q | TCTC | 9.7 |
| T152Q | GTTG | 9.64 |
| T152Q | TTGA | 9.56 |
| T152Q | TCTA | 9.37 |
| T152Q | ATTG | 9.12 |
| T152Q | TTAG | 9.09 |
| T152Q | TTCT | 9.04 |
| T152Q | ATTA | 8.96 |
| T152Q | TTGG | 8.65 |
| T152Q | TCCA | 8.57 |
| T152Q | TTTG | 8.56 |
| T152Q | ACTC | 8.52 |
| T152Q | GCCC | 8.37 |
| T152Q | TTTT | 8.32 |
| T152Q | CTTA | 7.83 |
| T152Q | TTCC | 7.74 |
| T152Q | TTCG | 7.35 |
| T152R | CTTC | 16.05 |
| T152R | CCTG | 14.82 |
| T152R | CCTC | 13.67 |
| T152R | GCCC | 12.97 |
| T152R | CCCC | 12.95 |
| T152R | GTTG | 12.67 |
| T152R | GCCG | 12.64 |
| T152R | ACTC | 12.49 |
| T152R | CCGC | 12.38 |
| T152R | CCGG | 12.22 |
| T152R | TCGA | 12.16 |
| T152R | CTGC | 11.86 |
| T152R | CCCG | 11.78 |
| T152R | TCGG | 11.59 |
| T152R | ATTA | 11.55 |
| T152R | CCCA | 11.27 |
| T152R | TCAG | 11.2 |
| T152R | TTCA | 11.03 |
| T152R | CTTA | 10.87 |
| T152R | CCTA | 10.82 |
| T152R | ATTG | 10.8 |
| T152R | TTCG | 10.71 |
| T152R | TCCG | 10.7 |
| T152R | ACGA | 10.69 |
| T152R | TCCA | 10.66 |
| T152R | ATTC | 10.62 |
| T152R | TCGT | 10.58 |
| T152R | GCAC | 10.54 |
| T152R | TTAC | 10.46 |
| T152R | TCTA | 10.44 |
| T152R | ACCC | 10.44 |
| T152R | GCTA | 10.41 |
| T152R | TTTT | 10.4 |
| T152R | GCCA | 10.38 |
| T152R | GTTA | 10.31 |
| T152R | ACTG | 10.25 |
| T152R | GCTG | 10.24 |
| T152R | GCGA | 10.23 |
| T152R | ACTA | 10.21 |
| T152R | TTGT | 10.12 |
| T152R | TTCT | 10.07 |
| T152R | GCTC | 10.05 |
| T152R | ACCG | 10.01 |
| T152R | CCGA | 9.99 |
| T152R | TCGC | 9.92 |
| T152R | GTTC | 9.85 |
| T152R | CTCG | 9.82 |
| T152R | GCGG | 9.74 |
| T152R | TTAG | 9.7 |
| T152R | TCCC | 9.66 |
| T152R | TCTC | 9.58 |
| T152R | ACCA | 9.58 |
| T152R | CTTG | 9.53 |
| T152R | TTGG | 9.53 |
| T152R | CTCA | 9.17 |
| T152R | TTGC | 9.03 |
| T152R | ACGG | 8.22 |
| T152W | TTGC | 16.19 |
| T152W | TTCA | 14.29 |
| T152W | TTCG | 13.39 |
| T152W | TTCC | 13.36 |
| T152W | TTTT | 13.32 |
| T152W | TCTA | 12.94 |
| T152W | TTTG | 12.53 |
| T152W | CTTC | 12.39 |
| T152W | CTTG | 12.21 |
| T152W | ATTA | 12.11 |
| T152W | TCCG | 12.04 |
| T152W | CCTG | 11.72 |
| T152W | CTTA | 11.67 |
| T152W | TTTC | 11.21 |
| T152W | TTTA | 11.1 |
| T152W | ATTG | 11.02 |
| T152W | CCTA | 10.57 |
| T152W | TCTC | 10.48 |
| T152W | TTAC | 10.29 |
| T152W | ATTC | 9.95 |
| T152W | GTTA | 9.86 |
| T152W | GTTC | 9.45 |
| T152Y | TCCA | 16.58 |
| T152Y | TCTG | 15.9 |
| T152Y | TCTC | 15.89 |
| T152Y | CCTC | 15.47 |
| T152Y | TCTA | 15.43 |
| T152Y | TTAC | 15.31 |
| T152Y | CCTG | 15.3 |
| T152Y | CTTG | 15.16 |
| T152Y | ACTG | 14.71 |
| T152Y | GCTA | 14.52 |
| T152Y | ATTA | 14.36 |
| T152Y | ACTC | 14.29 |
| T152Y | ATTG | 14.24 |
| T152Y | TTCG | 14.05 |
| T152Y | CCGC | 13.93 |
| T152Y | GCCC | 13.92 |
| T152Y | TCCG | 13.65 |
| T152Y | CTTC | 13.65 |
| T152Y | GCTC | 13.61 |
| T152Y | GTTG | 13.55 |
| T152Y | TCAC | 13.42 |
| T152Y | ATTC | 13.38 |
| T152Y | CTTA | 13.2 |
| T152Y | TTGC | 13.03 |
| T152Y | TCGC | 12.87 |
| T152Y | GTTA | 12.47 |
| T152Y | TCCC | 12.23 |
| T152Y | ACTA | 12.07 |
| T152Y | CCTA | 12.04 |
| T152Y | GCTG | 11.97 |
| T152Y | ACGC | 11.96 |
| T152Y | TTCA | 11.9 |
| T152Y | TTCC | 11.9 |
| T152Y | GTTC | 11.89 |
| T152Y | TCGG | 11.8 |
| T152Y | TTCT | 11.68 |
| T152Y | CCCC | 11.49 |
| T152Y | CCAC | 11.49 |
| T152Y | GCGC | 11.42 |
| T152Y | CCCA | 11.39 |
| T152Y | GCAC | 11.15 |
| T152Y | TTGA | 10.7 |
| T152Y | CTCC | 10.58 |
| T152Y | CCCG | 10.42 |
| T152Y | CTTT | 10.32 |
| T152Y | TTTT | 10.31 |
| T152Y | ACCC | 10.09 |
| T152Y | TTGG | 9.8 |
| T152Y | TTTC | 9.37 |
| T152Y | ACAC | 8.65 |
| V596H | TTTG | 11.75 |
| V596H | TTTC | 11.08 |
| V596T | CTTA | 20.31 |
| V596T | TTTG | 16.49 |
| V596T | TTCA | 15.21 |
| V596T | CTTG | 14.59 |
| V596T | TTTA | 14.55 |
| V596T | CTTC | 14.43 |
| V596T | TCTA | 13.11 |
| V596T | TTCC | 13 |
| V596T | TTGC | 12.93 |
| V596T | TCCC | 12.65 |
| V596T | TTTT | 12.27 |
| V596T | TCTC | 12.27 |
| V596T | TCTG | 12.07 |
| V596T | CCCC | 12.03 |
| V596T | TTTC | 12.02 |
| V596T | TTCG | 10.87 |
| V596T | CCCG | 9.96 |
| V596T | TCCG | 9.6 |
| V596T | GTTC | 9.3 |
| V596T | GCGC | 9.17 |
| W649H | TTTC | 11.36 |
| W649H | TTTA | 11.18 |
| W649H | TTTG | 10.47 |
| W649K | TTTA | 13.34 |
| W649R | TTTA | 8.84 |
| W649S | TTTA | 9.77 |
| W649Y | TTTC | 11.78 |
| W649Y | TTTG | 11.48 |
| W649Y | TTTA | 11.05 |
| Y542F | TTTA | 11.24 |
| Y542F | TTTC | 10.25 |
| Y542F | TTTG | 9.9 |
| Y542H | CTTA | 11.46 |
| Y542H | TTTT | 11.44 |
| Y542H | CTTC | 10.96 |
| Y542H | TTTC | 10.6 |
| Y542H | TCCC | 10.37 |
| Y542H | GTTA | 10.23 |
| Y542H | TTCG | 10.16 |
| Y542H | TTCA | 10.06 |
| Y542H | TTTG | 10.04 |
| Y542H | CCAC | 8.42 |
| Y542H | GCTC | 8.27 |
| Y542K | TTTA | 10.62 |
| Y542K | TTTG | 9.4 |
| Y542K | TTCA | 8.45 |
| Y542L | CTTA | 12.93 |
| Y542L | TTTA | 10.46 |
| Y542L | TTTC | 10.2 |
| Y542L | TTTG | 10.04 |
| Y542M | TTTG | 11.54 |
| Y542M | TTTA | 11.15 |
| Y542M | TTTC | 10.32 |
| Y542M | CTTG | 10.17 |
| Y542M | CTTA | 10.05 |
| Y542M | CTTC | 8.67 |
| Y542N | TTGC | 11.39 |
| Y542N | TTTA | 11.38 |
| Y542N | TTTG | 11.27 |
| Y542N | TTCC | 11.06 |
| Y542N | TTTT | 10.31 |
| Y542N | GTTC | 10.16 |
| Y542N | CCCC | 9.87 |
| Y542N | ATTA | 9.39 |
| Y542N | TCCC | 9.37 |
| Y542N | TTTC | 9.21 |
| Y542R | TTTT | 10.21 |
| Y542R | CTTG | 8.75 |
| Y542R | TTCA | 8.73 |
| Y542R | TCCC | 8.68 |
| Y542R | GTTA | 7.7 |
| Y542T | TTTA | 11.46 |
| Y542T | TTTC | 9.68 |
| Y542T | TTTG | 9.59 |
| Y542V | TTTG | 12.03 |
| Y542V | TTTA | 10.76 |
| Y616E | TTTG | 12.92 |
| Y616E | TTTA | 12.58 |
| Y616E | TTTC | 12.02 |
| Y616F | TTTT | 12.56 |
| Y616F | TTTA | 12.36 |
| Y616F | CTTA | 12.33 |
| Y616F | CTTG | 11.97 |
| Y616F | TTTG | 11.74 |
| Y616F | TTCA | 11.5 |
| Y616F | ATTG | 11.46 |
| Y616F | CTTC | 11.34 |
| Y616F | TTCC | 11.06 |
| Y616F | TCTA | 10.99 |
| Y616F | ATTA | 10.97 |
| Y616F | TTTC | 10.79 |
| Y616F | TTCT | 10.63 |
| Y616F | GTTG | 10.46 |
| Y616F | TTGC | 9.95 |
| Y616F | TTCG | 9.74 |
| Y616F | TCTG | 9.65 |
| Y616F | ATTC | 9.45 |
| Y616F | TTAC | 8.77 |
| Y616H | CTTA | 12.23 |
| Y616H | CTTG | 11.73 |
| Y616H | TTTC | 11.26 |
| Y616H | CTTC | 11.18 |
| Y616H | TTTT | 11.11 |
| Y616H | TTTA | 11 |
| Y616H | TTTG | 11 |
| Y616H | TTCA | 10.45 |
| Y616H | TTCC | 10.28 |
| Y616H | TTGC | 10.16 |
| Y616K | TTTG | 9.23 |
| Y616K | TTCC | 8.66 |
| Y616R | TTTG | 10.22 |
| Y616R | TTCC | 9.77 |
| Y646E | TTTA | 13.43 |
| Y646E | CTTA | 12.79 |
| Y646E | TTTT | 11.99 |
| Y646E | TTTG | 11.63 |
| Y646E | TTTC | 11.28 |
| Y646E | CTTC | 11.26 |
| Y646E | CTTG | 10.71 |
| Y646E | TTCC | 9.96 |
| Y646H | TTGC | 13.8 |
| Y646H | GTTC | 12.39 |
| Y646H | GTTA | 12.24 |
| Y646H | TCTA | 11.57 |
| Y646H | TCTC | 11.45 |
| Y646H | ATTA | 10.92 |
| Y646H | TTCT | 10.69 |
| Y646H | TCTG | 10.57 |
| Y646H | TCCC | 10.3 |
| Y646H | CTTC | 10.25 |
| Y646H | ATTG | 10.23 |
| Y646H | CTTG | 10.13 |
| Y646H | TTCC | 9.81 |
| Y646H | CCTG | 9.69 |
| Y646H | ATTC | 9.61 |
| Y646H | GTTG | 9.13 |
| Y646K | TTTT | 10.71 |
| Y646K | CTTA | 9.82 |
| Y646K | TTTC | 9.2 |
| Y646N | TTCA | 10.45 |
| Y646N | ATTA | 10.38 |
| Y646N | TTTA | 8.91 |
| Y646N | TCTA | 8.61 |
| Y646Q | ATTA | 11.69 |
| Y646Q | ATTG | 11.67 |
| Y646Q | TCTA | 11.39 |
| Y646Q | TTCC | 11.24 |
| Y646Q | CTTC | 10.99 |
| Y646Q | TTTT | 10.98 |
| Y646Q | TTCA | 10.58 |
| Y646Q | CTTA | 9.82 |
| Y646Q | TCTC | 9.52 |
| Y646Q | TTTG | 9.27 |
| Y646R | TTTC | 10.17 |
| Y646R | TCTA | 9.17 |
| Y646R | TTTA | 8.09 |
| Y646W | TTTT | 10.69 |
| Y646W | TTTC | 9.78 |
| Y646W | CTTG | 9.6 |

We observed 36 unique PAM sequences cleaved in vitro using two LbCas12a controls. This is in line with the observation that in vitro wtLbCas12a can recognize and cleave more sequences than just TTTV. TTCN, CTTN, TCTN, and others have been shown to be recognized and cleaved by LbCas12a in vitro, where AsCas12a was shown only to cleave TTTN (Zetsche et al. Cell 163:759-771 (2015)).

Some of the mutants increased the total number of PAM sequences recognition and cleavage in vitro (Table 5) as compared to wtLbCas12a. This does not speak to the absolute PAM recognition sequences, but to the overall promiscuity imparted by individual mutations. Some individual point mutants were more promiscuous than wildtype. For example, T152R recognized 57 different PAMs and K959Y recognized 45.

TABLE 5
Some of the more promiscuous LbCas12a mutants as judged by the
number of PAMs (nPAM) the recognized and cleaved in vitro.

| Mutant | nPAM |
|---|---|
| T152R | 57 |
| T152Y | 50 |
| T152K | 47 |
| D156Q | 45 |
| K595Y | 45 |
| D156L | 43 |
| D122H | 39 |
| K116R | 39 |
| D156K | 38 |
| K121S | 38 |
| K121R | 37 |
| wildtype | 36 |

### Comparing Depletions to Wildtype LbCas12a

In vitro wtLbCas12a can recognize and cleave more sequences than just TTTV (Zetsche et al. Cell 163:759-771 (2015)). TTCN, CTTN, TCTN, and others have been shown to be recognized and cleaved by LbCas12a in vitro, where AsCas12a was shown only to cleave TTTN (Zetsche et al. Cell 163:759-771 (2015)). The goal of this study was to expand LbCas12a's PAM recognition beyond its wildtype capabilities. To this end, we employed a different analysis than library depletion scores alone. Those scores are important for determining absolute PAM recognition and cleavage in vitro, but do not readily highlight the changes to the PAM recognition by the enzyme due to the introduced point mutations.

First, the 5 nucleotide depletion results were collapsed into 4 nucleotide PAMS, as before. Each time point was maintained individually. Each mutant-time point NGS total counts were normalized to 100 counts per PAM to account for loading differences on the NGS chip. Then the global medians for each 4 nt PAMs were compared to each mutant-time point. This provided a depletion as compared to wildtype, rather than a depletion as compared to the total library. The results highlight which mutations changed the PAM recognition profiles. We took a conservative approach and chose a depletion score of 4 or over as an indication of new PAM recognition by a mutant. A depletion score of 4 indicated four times as much of that particular PAM-containing library member was cleaved as compared to the median for wildtype. For example if 100 NGS counts were remaining for a PAM with GCGC for wildtype and 25 counts remained for a particular mutant-time point, then a score of 4 was calculated.

A summary of each of the 186 mutations is shown below in Table 6. Mutations provided in bold lettering indicates that the mutation recognized and cleaved more than 3 new PAM sequences as compared to wildtype with a score above 4. Mutations provided in italics indicates the mutant acquired between 1 and 3 or new PAM sequences as compared to wildtype with a score above 4. Mutations in regular font (not bolded or italicized) indicates the point mutation did not cleave new PAM sequences as compared to wildtype with a score above 4. Certain amino acids, such as T149 did not acquire new PAM recognitions despite being near the PAM-recognition domain of the protein and testing 10 new amino acids. Other amino acids, such as D156, appeared to be a hot-spot for engineering new PAM recognition motifs. Aspartate 156 when changed to 10 differing amino acids had with 7 mutations recognizing multiple new PAMs, 1 showing a few new PAMs, and two not acquiring new PAMs as compared to wtLbCAs12a. In general, any locations which showed a difference in PAM recognition and cleavage as compared to wildtype could be combined into double, triple, or multiple mutations to further alter PAM recognition. In total 130/186 point mutations did not acquire new PAMs over wtLbCas12a (regular font/not bold or italicized) above a score of 4, 40/186 acquired many new PAMs (bold font), and 16/186 acquired 1-3 new PAMs (italicized font). An overall 30% success rating (56/186) indicates an efficacious method was used to design novel PAM recognition motifs by making point mutations to LbCas12a.

TABLE 6
Summary table for the 186 LbCas12a point mutations (reference sequence SEQ ID NO: 1).

| | | | | | | | | | | | | | |
|---|---|---|---|---|---|---|---|---|---|---|---|---|---|
| K116R | K120R | K121S | D122R | E125R | T148H | T149A | T152R | D156R | E159K | Q529N | G532D | D535N | |
| K116N | K120H | K121T | D122K | E125K | T148S | T149C | T152K | D156K | E159R | Q529T | G532N | D535H | |
| | K120N | K121H | D122H | E125Q | T148A | T149S | T152W | D156Y | E159H | Q529H | G532S | D535V | |
| | K120T | K121R | D122E | E125Y | T148C | T149G | T152Y | D156W | E159Y | Q529A | G532H | D535T | |
| | K120Y | K121G | D122N | | | T149H | T152H | D156Q | E159Q | Q529F | G532F | D535S | |
| | K120Q | K121D | | | | T149P | T152Q | D156H | | Q529G | G532K | D535A | |
| | | K121Q | | | | T149F | T152E | D156I | | Q529S | G532R | D535W | |
| | | | | | | T149N | T152L | D156V | | Q529P | G532Q | D535K | |
| | | | | | | T149D | T152F | D156L | | Q529W | G532A | | |
| | | | | | | T149V | | D156E | | Q529D | G532L | | |
| | | | | | | | | | | | G532C | | |
| K538R | D541N | Y542R | L585G | K591W | M592R | K595R | V596T | S599G | K600R | K601R | Y616K | Y646R | W649H |
| K538V | D541H | Y542K | L585H | K591F | M592K | K595Q | V596H | S599H | K600H | K601H | Y616R | Y646E | W649K |
| K538Q | D541R | Y542H | L585F | K591Y | M592Q | K595Y | V596G | S599N | K600G | K601Q | Y616E | Y646K | W649R |
| K538W | D541K | Y542Q | | K591H | M592E | K595L | V596A | S599D | | K601T | Y616F | Y646H | W649Y |
| K538Y | D541Y | Y542F | | K591R | M592A | K595W | | | | | Y616H | Y646Q | W649E |
| K538F | D541I | Y542L | | K591S | | K595H | | | | | | Y646W | W649S |
| K538H | D541A | Y542M | | K591A | | K595E | | | | | | Y646N | W649V |
| K538L | D541S | Y542P | | K591G | | K595S | | | | | | | W649T |
| K538M | D541E | Y542V | | K591P | | K595D | | | | | | | |
| K538C | | Y542N | | | | K595M | | | | | | | |
| K538G | | Y542T | | | | | | | | | | | |
| K538A | | | | | | | | | | | | | |
| K538P | | | | | | | | | | | | | |

Many of the point mutations imparted novel PAM recognition to LbCas12a, allowing it to cleave DNA preceded by these sequences in vitro. Some mutations caused an increase in overall promiscuity, while others designed and tested were not shown to change wtLbCas12a recognition and cleavage. Overall 130/186 point mutations did not acquire new PAMs over wtLbCas12a (Table 6, regular font/not italicized or bold) above a score of 4, 40/186 acquired many new PAMs (Table 6, bold font), and 16/186 acquired 1-3 new PAMs (Table 6, italicized font). An overall 30% success rating (56/186) indicates an efficacious method was used to design novel PAM recognition motifs by making point mutations to LbCas12a.

### 2. Determining Binding of Point Mutations and Combinations in Prokaryotes

Combinations of individual mutations can alter the PAM recognition even more than single mutations. However, such experiments rapidly scale to large numbers of combinations to test. Taking just the 40 mutations which caused LbCas12a to recognize 3 or more new PAMs and making a library of double mutants a total of 40² or 1,600 enzymes could be tested. Making a triple-mutant library would result in 40³ or 64,000 enzymes to purify and assay in replicates, which is not practical. We, therefore, adapted a bacterial method, known as PAM-SCANR (Leenay et al. Mol Cell 62, 137-147 (2016)) to assess combinatorial mutations. We used a library in Escherichia coli to test binding of Cas12a mutations to the 256 possible PAM NNNN variants. This assay does not test cleavage, rather it tests binding in vivo. Since the mutations made were nowhere near the catalytic region, binding is expected to reflect cleavage as well (this was verified later in the 293T assay). The advantage to PAM-SCANR is the ability to rapidly test not only point mutations, but combinations of amino acid point mutations in a quick and accurate way. It also tends to be more stringent than in vitro cleavage assays.

### Reporter Plasmid

Plasmid pWISE1963 was used as the base vector for generating reporters with each of the 256 PAMS. The plasmid contains Spectinomycin resistance, ColE1 origin of replication, LacI, and eGFP under control of the lac promoter. 256 gene blocks containing the fragment between the NotI and SmaI restriction site (from just 5′ of the lacI promoter into the lacI gene) were synthesized by Twist Bioscience. Each fragment contained a different 4-mer PAM directly 5′ of the lad promoter. Each gene block was cloned into the pWISE1963 via restriction and ligation. Clones were selected for each variant and the identity of the PAM was verified by Sanger sequencing.

### CRISPR-Cas Plasmid

Plasmid pWISE2031 was used as the base vector for generating all CRISPR-Cas plasmids. The plasmid contains Chloramphenicol resistance, a CloDF13 origin of replication, dLbCas12a driven by promoter BbaJ23108, and the LbCas12a with a crRNA targeting the lacI promoter driven by the BbaJ23119 promoter. The negative control plasmid, pWISE1961 contains the same components as pWISE2031 with the exception that a non-targeting crRNA was used. Each point and combinatorial mutant (pWISE2984-pWISE3007) was constructed via site-directed mutagenesis of pWISE2031 at Genewiz.

### Cell Line

An E. coli cell line containing a chromosomal deletion of the lad gene, JW0336, was obtained from Dharmacon Horizon Discovery. Electrocompetent cells were prepared according to the protocol described (Sambrook, J., and Russell, D. W. (2006). Transformation of E. coli by Electroporation. Cold Spring Harb Protoc 2006, pdb.prot3933). E. coli JW0336 was used in all library transformation and cell sorting experiments.

### Preparation of Reporter Library

10 ng of each reporter plasmid described in the section above was pooled into a single tube to generate a library for transformation and amplification. 1/20^{th} of the pooled plasmid library (approximately 0.5 ng of each reporter) was transformed into supercompetent XL1-Blue according to manufacturer's instructions. After 1-hour of recovery at 37° C. with shaking at 225 rpm the entire transformation was transferred to 1-L of LB Spectinomycin and grown overnight at 37° C. with shaking at 225 rpm. The following day plasmid DNA was extracted from the overnight culture using a ZymoPURE plasmid gigaprep kit according to manufacturer's instructions. The DNA was quantified by nanodrop and used in all subsequent library transformations.

### Library Transformations and Cell Sorting

100 ng of reporter plasmid library and 100 ng of Crispr/Cas plasmid were co-transformed into 40 uL of JW0336 by electroporation. Transformations were recovered at 37° C. with shaking at 225 rpm for 1-hour. At the end of recovery, 10 uL of transformation was removed, mixed with 90 uL LB and plated onto LB agar plates with chloramphenicol and spectinomycin to determine transformation efficiency. The remaining amount of recovery (990 uL) was transferred to an overnight culture containing 29 mL of LB with Spectinomycin and Chloramphenicol. The cultures were grown overnight at 37° C. with shaking at 225 rpm. The following morning colonies were counted on the transformation plates to determine transformation efficiency; all transformations except for 2 of them showed >2,000 transformants, equating to 10× or greater coverage of the reporter library. The two samples that did not show 10× or greater coverage were repeated. Glycerol stocks of the overnight cultures were then prepared and stored at −80 C, and 6-mL of each culture was miniprepped using a Qiagen miniprep kit, according to manufacturer's instructions. These minipreps were labeled “pre-sort” and stored at 4° C.

One optical density (OD) of each overnight library culture was spun down in a tabletop microcentrifuge at 8,000 rpm, 4° C. for 5 minutes. The supernatant was pipetted off, and 1 mL of filter sterilized 1× PBS buffer was added to each tube. The pellets were carefully re-suspended by pipetting. The wash with 1× PBS was repeated 2 more times, and after the final re-suspension the cells (about 10⁸ cells per mL in 1× PBS) were placed on ice. Each sample was sorted on a Beckman-Coulter MoFlo XDP cell sorter. A negative control (WT-dLbCas12a+non-targeting crRNA+reporter library) and positive control (WT-dLbCas12a+targeting crRNA+reporter library) were used to set gating parameters for cell sorting. Samples were sorted on single-cell purity mode, with voltage 425, ssc voltage 535, fsc voltage (gain) of 4.0. The typical rate of sorting was about 4000 events/second. Each sample had a minimum of 1.0×10⁶ events; cell sorting was performed until 50,000 GFP-positive events were collected or the sample was depleted. In cases where the sample was depleted, a minimum of 200 GFP positive events were collected. GFP-positive events were collected into tubes containing 2-mL of LB with Spectinomycin and Chloramphenicol. Post-sort, the samples were diluted to 6-mL with additional LB with Spectinomycin and Chloramphenicol, and then grown overnight at 37° C. with shaking at 225 rpm.

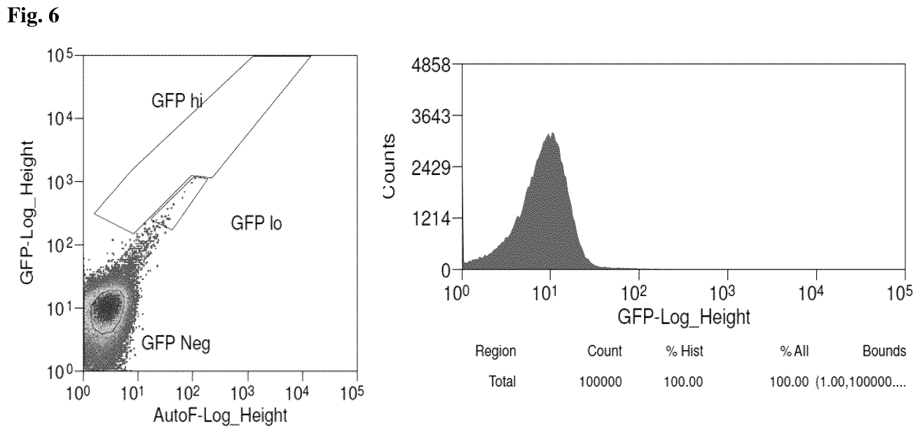

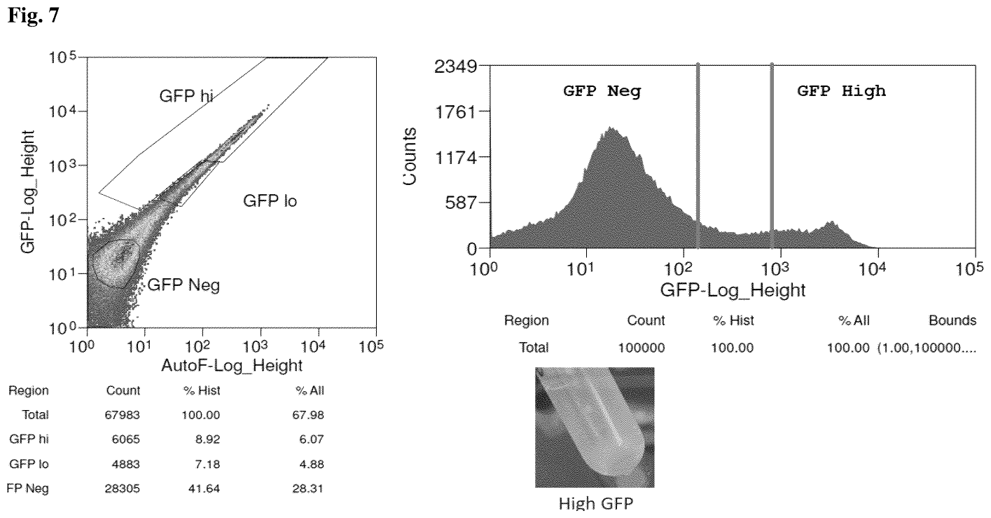

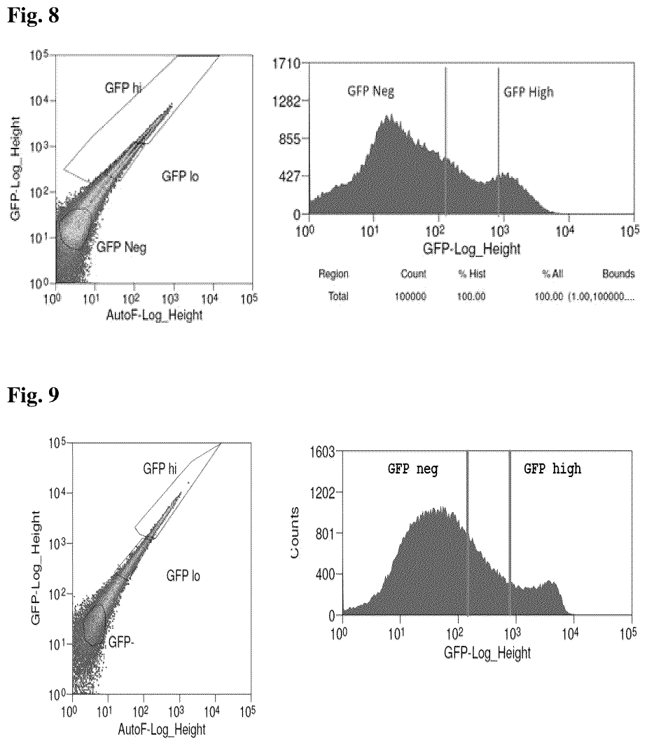

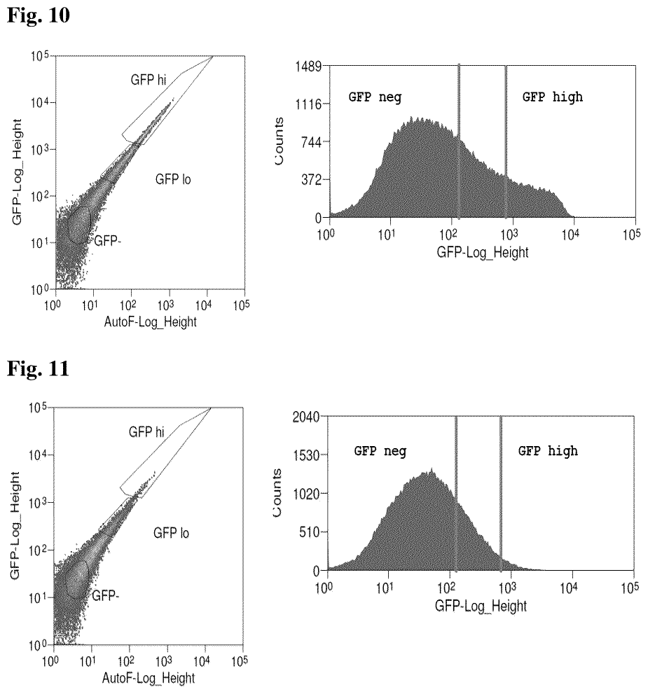

Example sorts are provided in FIGS. 6-11. FIG. 6 shows the cell sorting results of a negative control containing wtLbCas12a and a crRNA, which did not target the plasmid spacers. Sorted cells from the GFP high samples show no cells in the sorted fraction (left panel) and a single population of GFP signal, indicated by a single peak (right panel). FIG. 7 shows cell sorting results of wtLbCas12a and a crRNA targeting the plasmid spacers. Sorted cells from the GFP high samples show cells in the sorted fraction (left panel, GFP hi) and a two populations of GFP signal, indicated by two predominant peaks (right panel, GFP neg and GFP High). Also shown is the GFP high sorted cells fluorescing (lower right panel). FIG. 8 shows cell sorting results of LbCas12a-K595Y and a crRNA targeting the plasmid spacers. Sorted cells from the GFP high samples show cells in the sorted fraction (left panel, GFP hi) and a population indicated by two predominant peaks (right panel, GFP neg and GFP high). FIG. 9 shows the cell sorting results of LbCas12a-G532R-K595R double mutation control (Gao et al. Nat Biotechnol 35, nbt.3900 (2017)) and a crRNA targeting the plasmid spacers. Sorted cells from the GFP high samples show cells in the sorted fraction (left panel, GFP hi) and a population indicated by two predominant peaks (right panel). FIG. 10 shows cell sorting results of LbCas12a-T152R-K595Y double mutation, a combination of two of the point mutations used with a crRNA targeting the plasmid spacers. Sorted cells from the GFP high samples show cells in the sorted fraction (left panel, GFP hi) and a population indicated by two predominant peaks (right panel). FIG. 11 shows cell sorting results of LbCas12a-T152R-K538W-K595Y triple mutation, a combination of three of the point mutations used with a crRNA targeting the plasmid spacers. Sorted cells from the GFP high samples show cells in the sorted fraction (left, GFP hi) and a population indicated (right, green lines).

### Next Generation Sequencing

The following morning after sorting glycerol stocks of each overnight culture were prepared and stored at −80 C. The remaining amount of each 6-mL culture was miniprepped with a Qiagen miniprep kit according to manufacturer's instructions. These minipreps were labeled “post-sort” and were stored at 4° C. Pre-sort and post-sort minipreps were quantified by nanodrop, diluted 10-fold and handed off for sequencing on an Illumina Mi-Seq.

The spacer vectors were subjected to deep sequencing analysis to calculate the frequency of A/T/G/C at each position of PAM using an iliumina MiSeq according to manufacturer's protocols. Briefly, 10 ng of DNA was used as template for PCR. Phasing gene specific forward and reverse PCR primers were designed to amplify across the target site. Amplicon libraries were generated using a two-step PCR method, where primary PCR with 5′ tails allow a secondary PCR to add Illumina i5 and i7 adapter sequences and barcodes for sorting multiplexed samples. PCR amplifications were performed using the following parameters: 98° C. for 30 s; 25 cycles for PCR1 and 8 cycles for PCR2 (98° C. 10 s, 55° C. 20 s, 72° C. 30 s); 72° C. for 5 min; hold at 12° C. The PCR reactions were performed with Q5 High-Fidelity DNA Polymerase (New England BioLabs, Beverly, MA, United States). The secondary PCR amplicon samples were individually purified using AMPure XP beads according to manufacturer's instruction (Beckman Coulter, Brea, CA, United States); all purified samples were quantified using a plate reader, pooled with an equal molar ratio, and run on AATI fragment analyzer (Agilent Technologies, Palo Alto, CA, United States). The pooled amplicon libraries were sequenced on an Illumina MiSeq (2×250 paired end) using a MiSeq Reagent kit v2 (Illumina, San Diego, CA, United States).

### Sequencing Results of the Highly Fluorescent Sorted Cells

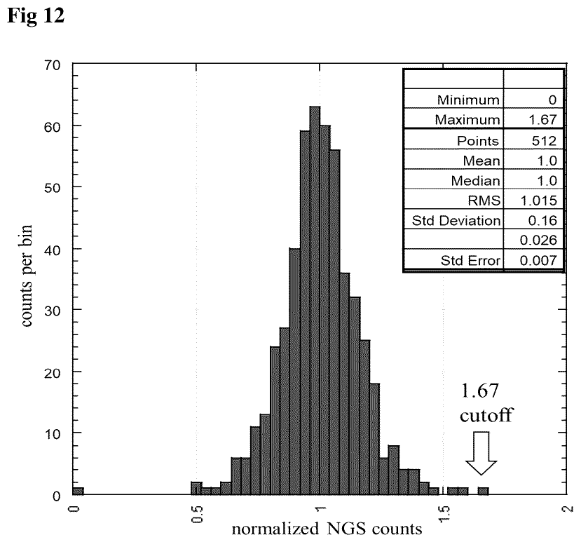

Two negative control samples were run containing wtdLbCas12a and a non-targeting crRNA in the presence of the 256-member reporter library. After normalization to 1.0, the value for each member of the library was plotted as a histogram (FIG. 12). FIG. 12 shows the total normalized NGS counts for two separate no crRNA controls and wildtype dLbCas12a and the reporter library. Two separate samples were analyzed and combined (total 512 points representing 256 PAMs×2). We chose the conservative 1.67 value, the highest count, as the cutoff for these experiments, above which we scored as PAM binding. The standard deviation was 0.16. Rather than choosing some multiplier of the standard deviation as a cutoff, we chose the absolute largest value found in either of the two negative controls, 1.67. This gave a highly stringent cutoff over 10 times the standard deviation of the data. In fact, only 3 PAM sequences were found above 1.5.

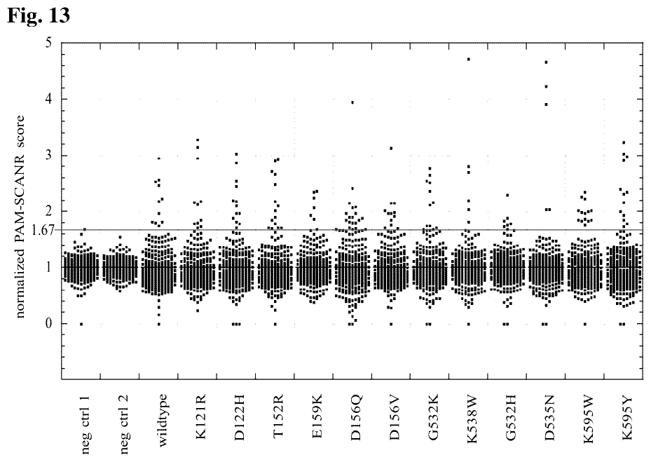

The pre-sorted pools were all sequenced prior to sorting. The average reads per PAM was from about 250 to 500 NGS reads, depending on the sample. The highly fluorescent post-sorted pools were sequenced and had similar read counts per PAM at about 250-500 reads per PAM. Both samples were then normalized to control for small loading differences in each NGS experiment. The two values were then subtracted and normalized to 1.0. Many PAM sequences were bound by the point mutation library above the 1.67 cutoff (FIG. 13, Table 7).

TABLE 7
The ability of point mutations to bind PAMs above the
1.67 threshold, sorted by normalized score.
TTTV sequences for wildtype are in bold.

| Wildtype | wildtype | T152R | T152R | K538W | K538W | K595Y | K595Y | D122H | D122H |
|---|---|---|---|---|---|---|---|---|---|
| PAM | score | PAM | Score | PAM | Score | PAM | Score | PAM | Score |
| TTTC | 2.96 | TTTG | 2.94 | TTTA | 4.71 | TCGC | 3.23 | TTTC | 3.03 |
| CTTG | 2.56 | TTTA | 2.9 | CTTA | 2.80 | TCCC | 3.03 | TTTG | 2.87 |
| TTTA | 2.54 | CTTA | 2.72 | AGCT | 2.70 | TTGC | 2.98 | TTTA | 2.56 |
| TTTG | 2.47 | TTTC | 2.66 | AATG | 2.19 | GCGC | 2.93 | TTTT | 2.46 |
| CCGC | 2.23 | GTTA | 2.48 | CTTC | 2.03 | ACGC | 2.40 | CTTA | 2.20 |
| CTTA | 2.19 | CTTG | 2.19 | TTTC | 1.82 | TCCA | 2.35 | TTCC | 2.19 |
| CTTC | 1.97 | GTTG | 2.1 | | | TTCC | 2.32 | CTTG | 2.10 |
| GTTC | 1.82 | CTTC | 2.02 | | | TCCG | 2.17 | GTTA | 1.97 |
| TTCC | 1.82 | AGAG | 1.91 | | | TTCG | 2.01 | ATTG | 1.96 |
| GTTG | 1.80 | ATTG | 1.85 | | | TCGG | 1.93 | CTTC | 1.89 |
| GTTA | 1.75 | TCCA | 1.79 | | | CCGC | 1.84 | TCTG | 1.82 |
| | | GCCA | 1.72 | | | CCCA | 1.79 | CCCA | 1.79 |
| | | TCTA | 1.72 | | | ACCC | 1.74 | GTTG | 1.72 |
| | | TTTT | 1.72 | | | | | | |
| | | GTTC | 1.71 | | | | | | |
| D156V | D156V | D156Q | D156Q | D535N | D535N | E159K | E159K | G532H | G532H |
| PAM | Score | PAM | Score | PAM | Score | PAM | Score | PAM | Score |
| AATG | 3.13 | TGTG | 3.96 | TTTG | 4.66 | TTTG | 2.37 | AATG | 2.30 |
| GTTG | 2.16 | CCTC | 2.42 | TTTC | 4.23 | TTTC | 2.36 | CCTA | 1.89 |
| TTTT | 2.15 | CCCA | 2.14 | TTTA | 3.91 | TTTA | 2.25 | GTTG | 1.83 |
| TTTA | 2.02 | GTTG | 2.09 | TTTT | 2.04 | CTTA | 2.07 | GTTA | 1.79 |
| CTTA | 1.99 | GTTC | 2.07 | CACG | 2.03 | CTTG | 1.94 | TTTA | 1.77 |
| GTTA | 1.98 | CTTC | 1.97 | | | CTTC | 1.86 | TCTA | 1.73 |
| ATTA | 1.97 | ATTC | 1.96 | | | CCCA | 1.68 | CTTA | 1.68 |
| CTTG | 1.93 | TTCC | 1.90 | | | GTTA | 1.67 | | |
| GTTC | 1.85 | TTGC | 1.90 | | | | | | |
| CTTC | 1.77 | TTCA | 1.88 | | | | | | |
| TCTC | 1.71 | ACTC | 1.86 | | | | | | |
| TTCA | 1.70 | ACAC | 1.85 | | | | | | |
| | | CTTG | 1.82 | | | | | | |
| | | AGCT | 1.80 | | | | | | |
| | | AATG | 1.80 | | | | | | |
| | | AAGC | 1.72 | | | | | | |
| | | GTAC | 1.70 | | | | | | |
| | | TCGC | 1.69 | | | | | | |
| | | CCTG | 1.69 | | | | | | |
| | | GTTA | 1.69 | | | | | | |
| | | TCCC | 1.67 | | | | | | |
| G532K | G532K | K121R | K121R | K595W | K595W | K538W | K538W | D535N | D535N |
| PAM | Score | PAM | Score | PAM | Score | PAM | Score | PAM | Score |
| TTTA | 2.76 | TTTG | 3.28 | TTTG | 2.35 | TTTA | 11.18 | GCGG | 18.70 |
| TTTC | 2.66 | TTTA | 3.15 | TTCC | 2.27 | CTTA | 9.57 | TTTG | 16.34 |
| TTTG | 2.56 | TTTC | 2.95 | TTTA | 2.23 | TTTC | 8.45 | TTTA | 13.15 |
| CCGC | 2.51 | CTTG | 2.18 | GCGC | 2.03 | TTTG | 7.27 | TTTC | 3.87 |
| TTCC | 2.37 | CTTA | 2.17 | TTCG | 2.02 | CTTC | 6.70 | TTGG | 3.83 |
| TTCA | 2.16 | TTTT | 2.14 | TTTC | 2.01 | CTTG | 6.33 | TTAC | 2.94 |
| TCCC | 2.13 | CTTC | 1.85 | TTCA | 1.99 | TATC | 5.35 | TCAC | 2.78 |
| TCCA | 1.76 | GTTG | 1.80 | ACCG | 1.97 | TATA | 4.90 | TTGC | 2.38 |
| GTTA | 1.75 | GTTA | 1.78 | TCCG | 1.87 | TATG | 4.39 | TTCC | 1.87 |
| ATTG | 1.72 | TTGG | 1.74 | TCGC | 1.85 | AATG | 2.75 | CCAC | 1.87 |
| CCCC | 1.69 | TTCC | 1.71 | TCCC | 1.84 | CCGC | 2.19 | TCGC | 1.84 |
| CTTA | 1.68 | | | AACG | 1.84 | AATC | 2.13 | CTTG | 1.67 |
| | | | | TTGC | 1.83 | GTTA | 1.89 | TTTT | 1.67 |
| | | | | TCCA | 1.76 | AATA | 1.68 | | |

We combined the three point mutations T152R, K538W, and K595Y in various combinations to make double and triple dLbCas12a mutants (T152R+K538W, K538W+K595Y, and T152R+K538W+K595Y). This was compared to a previously described control, which was developed in AsCas12a known as ‘RR’ whose LbCas12a mutations correspond to G532R+K595R control (Gao et al. Nat Biotechnol 35(8):789-792 (2017)). ‘RR’ have been described as being able to cause INDELs in TYCV+CCCC sequences in AsCas12a and subsequently LbCas12a.

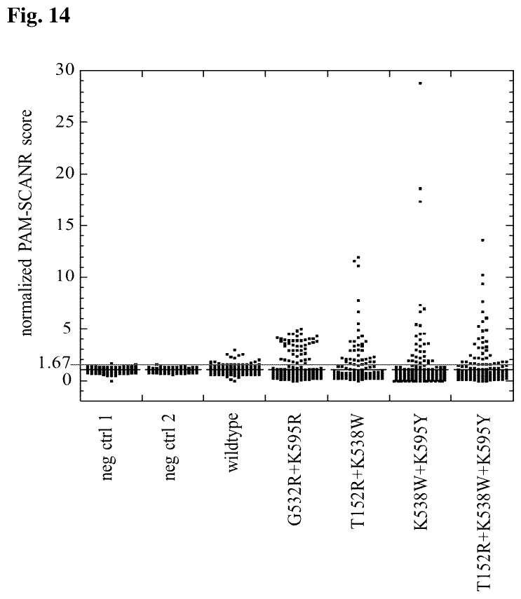

The same methodology was applied to score the combinatorial mutations. Pre and post-sorted pools were sequenced each having an average of about 250-500 MiSeq NGS reads per PAM library member. The pre and post-sort pools were normalized and subtracted, the difference being normalized to 1.0. Many PAM sequences were bound by the combinations above the 1.67 cutoff (FIG. 14, Table 8).

TABLE 8
Ability of combinatorial mutations to bind PAMs above the 1.67 threshold, sorted by
normalized score. TTTV sequences for wildtype are in bold. TYCV + CCCC sequences for
the G532R + K595R control are underlined.

| | | G532R + | G532R + | T152R + | T152R + | K538W + | K538W + | T152R + | T152R + |
|---|---|---|---|---|---|---|---|---|---|
| wildtype | wildtype | K595R | K595R | K538W | K538W | K595Y | K595Y | K538W + | K538W + |
| PAM | score | PAM | Score | PAM | Score | PAM | Score | K595Y PAM | K595Y Score |
| TTTC | 2.96 | TTTT | 5.07 | TTTC | 11.96 | TTTC | 11.96 | AGCC | 13.65 |
| CTTG | 2.56 | TTCG | 4.81 | TTTA | 11.62 | TTTA | 11.62 | TGGC | 10.28 |
| TTTA | 2.54 | ACCC | 4.52 | TTTG | 11.19 | TTTG | 11.19 | TACC | 9.35 |
| TTTG | 2.47 | TCCG | 4.51 | GTTG | 7.75 | GTTG | 7.75 | TGCC | 7.68 |
| CCGC | 2.23 | TTCA | 4.46 | GTTA | 6.68 | GTTA | 6.68 | CTGC | 6.69 |
| CTTA | 2.19 | TTTA | 4.30 | CTTG | 5.53 | CTTG | 5.53 | GGGC | 6.09 |
| CTTC | 1.97 | TTTG | 4.28 | CTTA | 4.93 | CTTA | 4.93 | GGCC | 5.71 |
| GTTC | 1.82 | GCCG | 4.15 | GTTC | 4.34 | GTTC | 4.34 | AGGC | 5.26 |
| TTCC | 1.82 | TTCT | 4.07 | TTCC | 4.11 | TTCC | 4.11 | TAGC | 4.96 |
| GTTG | 1.80 | CTCC | 4.03 | TCCC | 3.82 | TCCC | 3.82 | CGGC | 4.89 |
| GTTA | 1.75 | GCCA | 4.01 | TTCA | 3.78 | TTCA | 3.78 | ACCC | 4.15 |
| | | CCCG | 4.00 | ATTG | 3.78 | ATTG | 3.78 | GACC | 4.08 |
| | | TCCA | 3.92 | TCCG | 3.48 | TCCG | 3.48 | AAGC | 3.81 |
| | | ACTA | 3.90 | TTGG | 3.43 | TTGG | 3.43 | AGCA | 3.69 |
| | | GTCC | 3.87 | GCCC | 3.32 | GCCC | 3.32 | GCGC | 3.49 |
| | | CCCC | 3.87 | CTTC | 3.30 | CTTC | 3.30 | TACA | 3.26 |
| | | TTTC | 3.84 | CCCC | 2.95 | CCCC | 2.95 | TCCC | 3.17 |
| | | CCCA | 3.78 | TCTA | 2.88 | TCTA | 2.88 | CCCA | 3.15 |
| | | TCTA | 3.75 | GCCA | 2.86 | GCCA | 2.86 | TGCA | 2.87 |
| | | TCCT | 3.68 | TCCA | 2.86 | TCCA | 2.86 | GCCC | 2.66 |
| | | GCTA | 3.63 | ATTA | 2.44 | ATTA | 2.44 | CCCC | 2.49 |
| | | GCCC | 3.59 | CTCC | 2.29 | CTCC | 2.29 | TTGC | 2.42 |
| | | GTTA | 3.58 | CCCG | 2.19 | CCCG | 2.19 | ACGC | 2.21 |
| | | ACCA | 3.43 | ACCC | 2.16 | ACCC | 2.16 | GGCA | 2.21 |
| | | TCCC | 3.38 | TTCG | 2.13 | TTCG | 2.13 | ACCA | 2.18 |
| | | TTCC | 3.36 | GTCC | 2.13 | GTCC | 2.13 | AACC | 2.09 |
| | | ACCG | 3.36 | CCCA | 2.06 | CCCA | 2.06 | GACA | 1.83 |
| | | TCTG | 3.31 | GCCG | 1.99 | GCCG | 1.99 | CAGC | 1.81 |
| | | TCTT | 3.05 | GCTA | 1.97 | GCTA | 1.97 | ACCG | 1.80 |
| | | CTTA | 2.92 | TCTG | 1.96 | TCTG | 1.96 | GGGG | 1.80 |
| | | TCTC | 2.89 | TCTC | 1.84 | TCTC | 1.84 | TACG | 1.72 |
| | | CCCT | 2.89 | TTTT | 1.81 | TTTT | 1.81 | TTCC | 1.69 |
| | | CTCA | 2.86 | ATTC | 1.79 | ATTC | 1.79 | | |
| | | GTCA | 2.63 | GTCA | 1.67 | | | | |
| | | GTCG | 2.54 | | | | | | |
| | | CCTA | 2.54 | | | | | | |
| | | ATCC | 2.34 | | | | | | |
| | | GTTC | 2.11 | | | | | | |
| | | TTAC | 2.10 | | | | | | |
| | | CTCG | 2.03 | | | | | | |
| | | ATTA | 2.01 | | | | | | |
| | | GCTC | 1.94 | | | | | | |
| | | GCCT | 1.89 | | | | | | |
| | | CCTC | 1.82 | | | | | | |
| | | GCTT | 1.71 | | | | | | |

### Overall Analysis of PAM-SCANR Data

Wildtype LbCas12a showed strong TTTV binding the LbCas12a-G532R-K595R control showed strong TYCV and CCCC binding. This mutation, called ‘RR’ was developed in AsCas12a and shown to bind TYCV and CCCC (Gao et al. Nat Biotechnol 35(8):789-792 (2017)). However, in vitro wtLbCas12a can recognize and cleave TTCN, CTTN, TCTN, and others, where AsCas12a was shown only to cleave TTTN (Zetsche et al., Cell 163:759-771 (2015)). We deduced that the ‘RR’ mutation placed in the LbCas12a context would be more promiscuous than when it is placed in the AsCas12a context and this was what was observed for this control, with LbCas12a-RR recognizing 45 sequences. Both the wildtype and LbCas12a-RR results demonstrate the validity of the selection and sorting parameters

The mutations tested clearly indicated that novel PAMs are recognized in vivo by individual point mutations identified in vitro. For example, K595Y bound 13 PAMs above the 1.67 threshold, 11 of which were not recognized by wtLbCas12a, and none of which contained the TTTV sequence known to be bound by Cas12a. Similarly, T152R recognized 15 distinct PAMs, however in this case it retained the TTTV of wildtype. Overall out of the 12 point mutations tested, each had novel PAM binding sequences outside of the canonical TTTV motif and distinct from dLbCas12a controls.

### Effects of Combinations

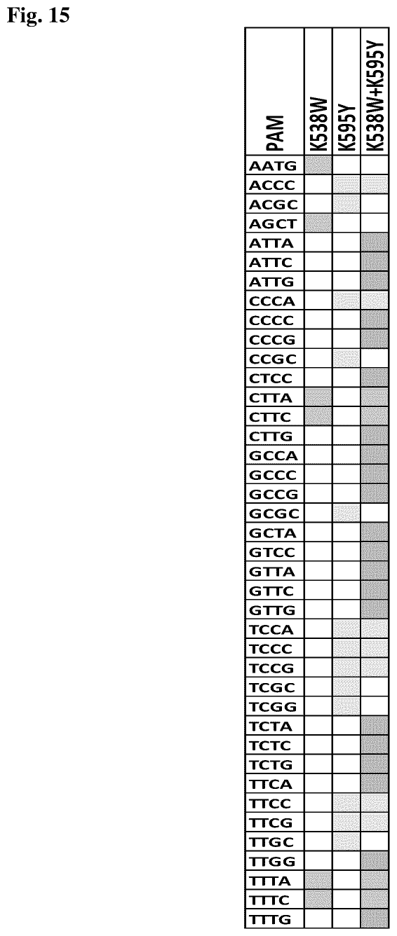

We found that combining multiple point mutations did not lead to a linear addition of the PAM sequences of the point mutations (FIG. 15). For example, combining LbCas12a point mutants K538W and K595Y result in an enzyme LbCas12a-K538W-K595Y which in some cases shares PAM recognition motifs with K538W (vertical hatched) or K595Y (horizontal hatched), but more often results in completely novel PAM recognition sequences (thatched). Using the same example, K538W recognizes AGCT, however K538W+K595Y does not. K595Y recognizes ACGC, however K538W+K595Y does not. CCCC is neither recognized by K538W nor K595Y, yet the double mutant binds it with high affinity.

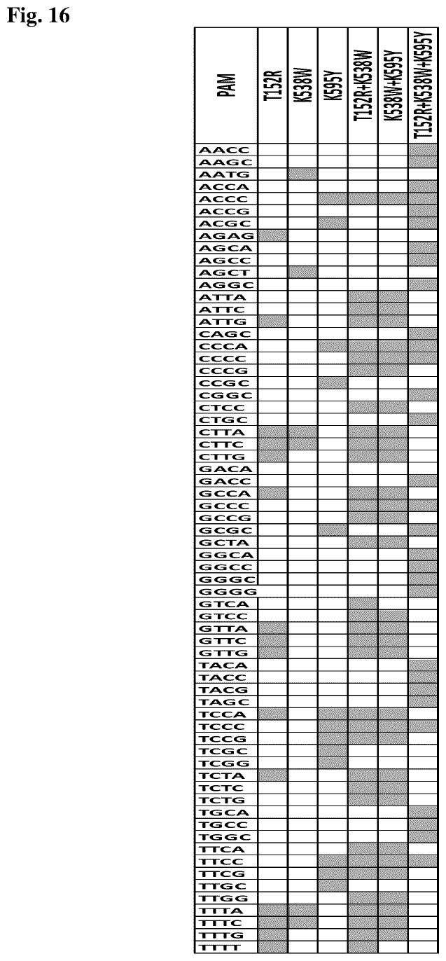

Overall, the combination of mutations results in a more than linear expansion of PAM recognition. For example K538W recognizes 6 PAM sequences, K595Y recognizes 13, but together they recognize 32 sequences (FIG. 15). A simple additive effect would result in 19 PAMs for the double mutant, rather than the 32 we observe. Furthermore, only 11 of the 32 sequences recognized by the double mutant are recognized by either of the two single mutations. A similar pattern is observed when combining three mutations (FIG. 16). The combination of T152R, K538W, and K595Y result in a triple mutation with different PAM recognitions than any of the three individual mutations alone. For example: GGCA, GGCC, GGGC, and GGGG only are recognized when all three mutations are made on LbCas12a. None of these PAMs are bound by any of the single or double mutations, but only when T152R, K538W, and K595Y are all mutated together.

### Comparing Point Mutations PAM Recognition in PAM-SCANR Versus In Vitro PAMDA

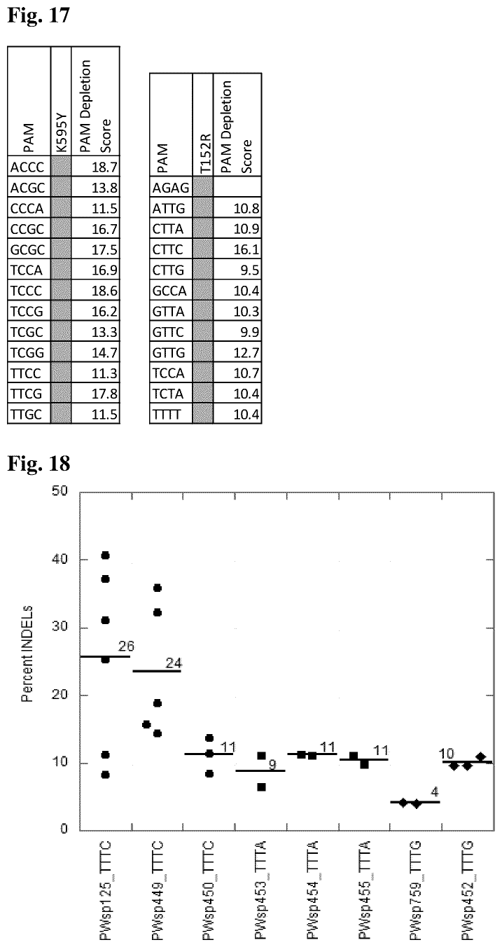

Taken as a whole for the 12 point mutations tested, PAM-SCANR hits above 1.67 were well represented in the in vitro PAMDA depletion assay. K595Y and T152R examples are shown (FIG. 17). FIG. 17 compares all non-TTTV PAMs which showed above a 1.67 score from PAN-SCANR (grey boxes) to K595Y (left panel) and T152R (right panel). All but one of the PAM-SCANR positive PAMs above the 1.67 cutoff had PAM depletion scores above the 9.2 cutoff in vitro. However, the PAM-SCANR method and analysis were more stringent than in vitro assays and analyses. For example, 13 different PAMs were sorted, sequences, and normalized to have values above 1.67 in PAM-SCANR. That is contrasted to the PAMDA assay which identified 45 sequences as readily cleaved in vitro. This is likely a function of relative concentrations inside of cells versus in test tubes, but it could also be a function of setting too stringent of a cutoff for PAM-SCANR or too permissive of a cutoff for PAMDA assays.

There is correlation between the datasets indicating our engineering of residues far from the catalytic site is affecting PAM recognition and binding and not catalysis. If mutations at these residues had affected nuclease activity along with PAM binding then there would be many hits in the PAM-SCANR assay (which measures binding but not cleavage) which did not show cleavage in the PAMDA assay. We do not see that pattern. We observe mutations which affected a change in binding (PAM-SCANR) also resulted were cleaved in vitro (PAMDA).

### 3. Determining Binding, Cleavage, and INDEL Formation in Eukaryotes

We chose three mutations T152R, K538W, and K595Y to test their ability to cause insertions or deletions (INDELs) in eukaryotic HEK293T cells. This assay gives valuable eukaryotic INDEL data. In order to get insertions and deletions in eukaryotes a number of criteria all have to be met: the CRISPR enzyme needs to be expressed and stable in the cell, the crRNA needs to be expressed and correctly processed, the protein:RNA complex needs to form, the complex needs to be stable, the complex needs to translocate in sufficient quantities into the nucleus, the target DNA needs to be accessible, the DNA has to be well-targeted by the particular guide-RNA design, and double-stranded breaks need to occur at a rate high enough to yield the occasional DNA repair mistake via an insertion or deletion (INDEL). This makes eukaryotic assays the most stringent assays in this study. A few dozen PAMs were tested for each of the 3 point mutants described below, rather than all 256 due to the experiment being low-throughput. Three different targets were chosen for each PAM-mutant combination to try to avoid false-negatives since often a particular guide is ineffective due to target accessibility.

### HEK293T Cell Testing

Eukaryotic HEK293T (ATCC CRL-3216) cells were cultured in Dulbecco's Modified Eagle's Medium plus GlutaMax (ThermoFisher) supplemented with 10% (v/v) FBS (FBS), at 37° C. with 5% CO2. Wildtype and mutant LbCas12 were synthesized using solid-state synthesis and subsequently cloned into plasmids behind a CMV promoter. CRISPR RNAs (crRNAs) were cloned behind a human U6 promoter (Table 9). HEK293T cells were seeded on 48-well collagen-coated BioCoat plates (Corning). Cells were transfected at about 70% confluency. 750 ng of protein plasmid and 250 ng of crRNA expression plasmids were transfected using 1.5 μl of Lipofectamine 3000 (ThermoFisher Scientific) per well according to the manufacturer's protocol. Genomic DNA from transfected cells were obtained after 3 days and indels were detected and quantified using high-throughput Illumina amplicon sequencing.

The spacer vectors were subjected to deep sequencing analysis to calculate the frequency of A/T/G/C at each position of PAM using an Illumina MiSeq according to manufacturer's protocols. Briefly, 10 ng of DNA was used as template for PCR. Phasing gene specific forward and reverse PCR primers were designed to amplify across the target site. Amplicon libraries were generated using a two-step PCR method, where primary PCR with 5′ tails allow a secondary PCR to add Illumina i5 and i7 adapter sequences and barcodes for sorting multiplexed samples. PCR amplifications were performed using the following parameters: 98° C. for 30 s; 25 cycles for PCR1 and 8 cycles for PCR2 (98° C. 10 s, 55° C. 20 s, 72° C. 30 s); 72° C. for 5 min; hold at 12° C. The PCR reactions were performed with Q5 High-Fidelity DNA Polymerase (New England BioLabs, Beverly, MA, United States). The secondary PCR amplicon samples were individually purified using AMPure XP beads according to manufacturer's instruction (Beckman Coulter, Brea, CA, United States); all purified samples were quantified using a plate reader, pooled with an equal molar ratio, and run on AATI fragment analyzer (Agilent Technologies, Palo Alto, CA, United States). The pooled amplicon libraries were sequenced on an Illumina MiSeq (2×250 paired end) using a MiSeq Reagent kit v2 (Illumina, San Diego, CA, United States).

### Wildtype Control

Wildtype LbCas12a (wtLbCas12a) recognizes TTTV (TTTA, TTTC, and TTTG). We used crRNA spacers (Table 9) to test the wildtype protein against TTTV containing 23 nucleotide spacer targets (FIG. 18).

TABLE 9
Spacers and targets

| | | | | SEQ |
|---|---|---|---|---|
| Spacer | HEK | | | ID |
| name | target | PAM | spacer sequence | NO |
| PWsp125 | EMX1 | TTTC | TCATCTGTGCCCCTCCCTCCCTG | 54 |
| PWsp449 | FANCF1 | TTTC | GCGGATGTTCCAATCAGTACGCA | 55 |
| PWsp450 | HEK2 | TTTC | CAGCCCGCTGGCCCTGTAAAGGA | 56 |
| PWsp453 | RNF2 | TTTA | TATGAGTTACAACGAACACCTCA | 57 |
| PWsp454 | RNF2 | TTTA | CACGTCTCATATGCCCCTTGGCA | 58 |
| PWsp455 | RNF2 | TTTA | GAACATGAAAACTTAAATAGAAC | 59 |
| PWsp759 | DNMT1 | TTTG | GTCAGGTTGGCTGCTGGGCTGGC | 60 |
| PWsp452 | HEK2 | TTTG | ACAGATGGGGCTGGACAATTTTT | 61 |

### Choice of Proteins and Targets

There were many point mutations which showed increased PAM accessibility in the PAMDA in vitro assay. Testing all of the efficacious PAM mutants against endogenous 293T cell targets is effectively impossible given our many point mutations and 256 possible 4 nucleotide (nt) PAMS due to the complexity, cost, and time of the experiment. We, therefore, chose three point mutations to test against a subset of PAMs. The three point mutations tested were T152R, K538W, and K595Y.

Genomic targets in 293T cells were chosen based on their PAM sequences. Genomic targets for the three point mutations were chosen at random using no particular rules other than having the appropriate 4 nt PAM and selecting 23 nucleotides downstream from that PAM. Three different spacers were chosen to assay each PAM. This is to account for the observation that activities of CRISPR enzymes is target-specific and often cannot be predicted.

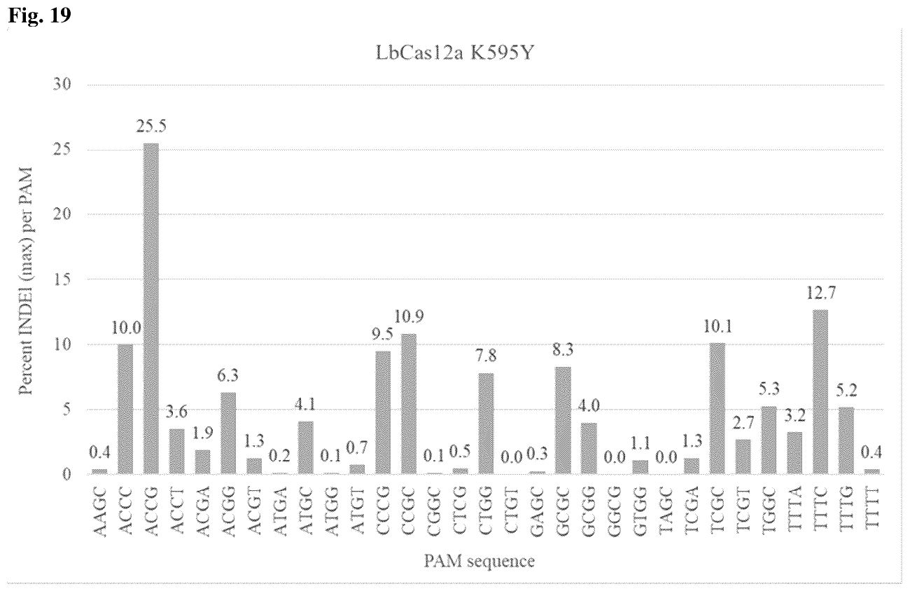

On average, we have observed that about half of 23 nucleotide wtLbCas12a spacers tested are ineffective despite having correct PAM TTTV sequences. With only three data points for each PAM and the observation that about 50% of targets will not produce INDELs, it is more informative to assess PAM recognition by visualizing maximum INDEL percentage per PAM rather than an average for randomly designed spacers (FIGS. 19-21). If a larger number of spacers for each PAM were assayed, then statistical tests may be used to assess their average editing efficiency.

Our overall transfection and assay conditions result in wildtype LbCAs12a causing INDELs in HEK293T cells at approximately 11-26% for TTTC, and 10% for TTTA, and 4-10% for TTTG (FIG. 18). These are pre-defined HEK293T targets sites and guides from the literature and, thus, are expected to be more effective than any randomly chosen guide. Despite a random design for the crRNA guides for the mutant, many new Cas12a PAM recognition sites caused INDELs at rates similar to wildtype at TTTV sequences (FIGS. 19-21). Any INDELs above 0.1% are above the noise of the sequencing assay which were read at 10,000 NGS read-depth.

K595Y was able to cause INDELs at ACCG at 25.5%, CCGC at 10.9%, TCGC at 10.1%, CCCG at 9.5%, GCGC at 8.3%, CTGG at 7.8%, ACGG at 6.3%, CCCG at 6.0%, TGGC at 5.3%, and others (FIG. 19). These numbers are all within the range of the TTTV controls for wtLbCas12a despite being randomly designed. One major hallmark of Cas12a proteins is that they recognize T-rich PAMs (Zetsche et al. Cell 163:759-771 (2015)). This limits their utility in genome editing technologies. K595Y clearly prefers C and G-rich PAMs, which will expand Cas2a utility to targets previously predominantly targets of Cas9 CRISPR enzymes which utilize G-rich PAMs (Jinek et al. Science 337, 816-821 (2012)). Only 31 of the total 256 possible four nucleotide PAMs were tested in 293T cells (or 12%) for K595Y. There are likely many other PAMs which could be recognized by K595Y and cause INDELs in eukaryotic cells.

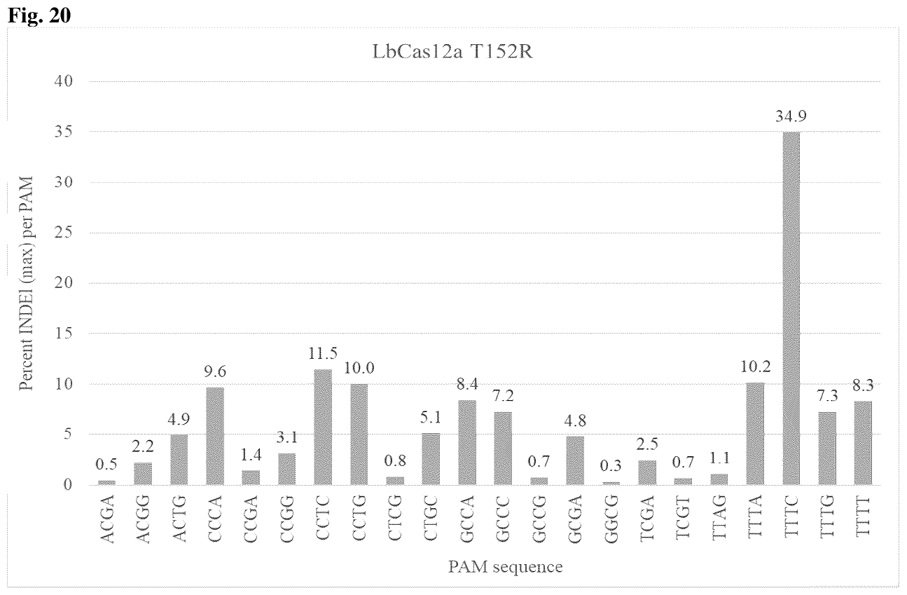

T152R was able to cause INDELs at CCTC at 11.5%, CCTG at 10.0%, CCCA at 9.6%, GCCA at 8.4%, GCCC at 7.2%, CTGC at 5.1%, and others (FIG. 20). Interestingly, T152R retained the TTTV recognition of wtLbCas12a by causing INDELs at TTTC at 34.9%, TTTA at 10.2%, and TTTG at 6.2%. It also picked up a TTTT recognition, causing INDELs at 8.3%. Only 22 of the total 256 possible four nucleotide PAMs were tested in 293T cells (or 9%) for T152R. There are likely many other PAMs which could be recognized by T152R and cause INDELs in eukaryotic cells.

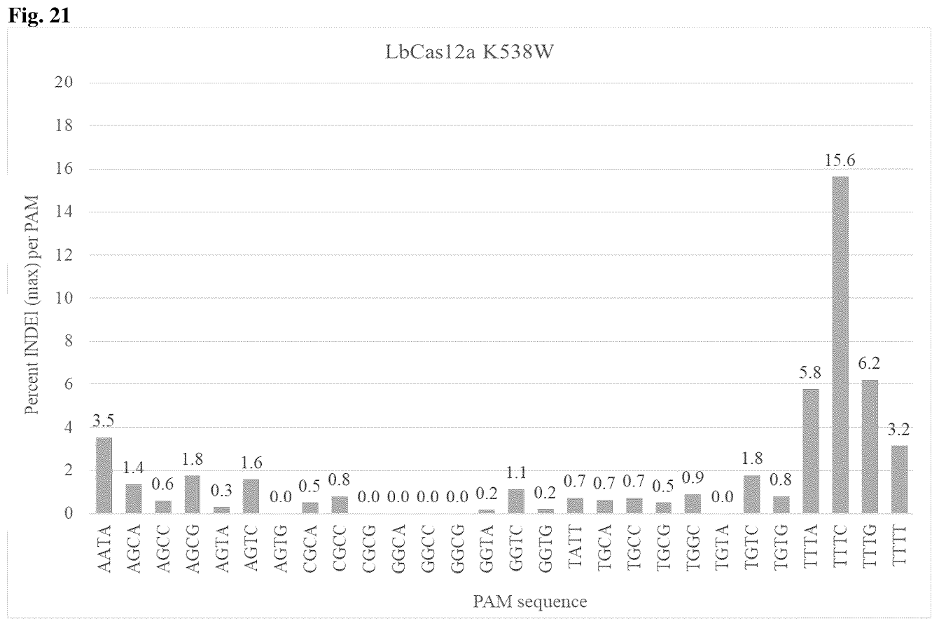

As shown in FIG. 21, 22 out of the 28 PAM targets did have activity above the background of 0.1%, indicating 79% of the PAMs tested were recognized and cleaved by this enzyme, although at times lower than may be desired for some applications. Six PAMs tested did not have any editing above background for the three targets chosen. The three TTTV targets all still had good activity with 15.6%, 6.2%, and 5.8% for TTTC, TTTG, and TTTA, respectively. Other PAM sequences with over 1% INDEL formation included ATTA (3.5%), TTTT (3.2%), TGTC (1.8%), AGCG (1.8%), AGTC (1.6%), AGCA (1.4%), and GGTC (1.1%). This point mutation was used in combination with T152R and/or K595Y in the PAM-SCANR experiments to generate a wide variety of PAM recognition, however, on its own it bound relatively few PAMs using that assay. It may be an excellent choice to use in future double mutations rather than alone to generate INDELs in HEK293T cells. Similar to the other two point mutations, only 28 out of the possible 256 four nucleotide PAMs were tested (11%) and it is possible that this mutant may recognize PAMs or targets not tested here.

### Correlation Between HEK293T INDELs and PAM-SCANR Binding

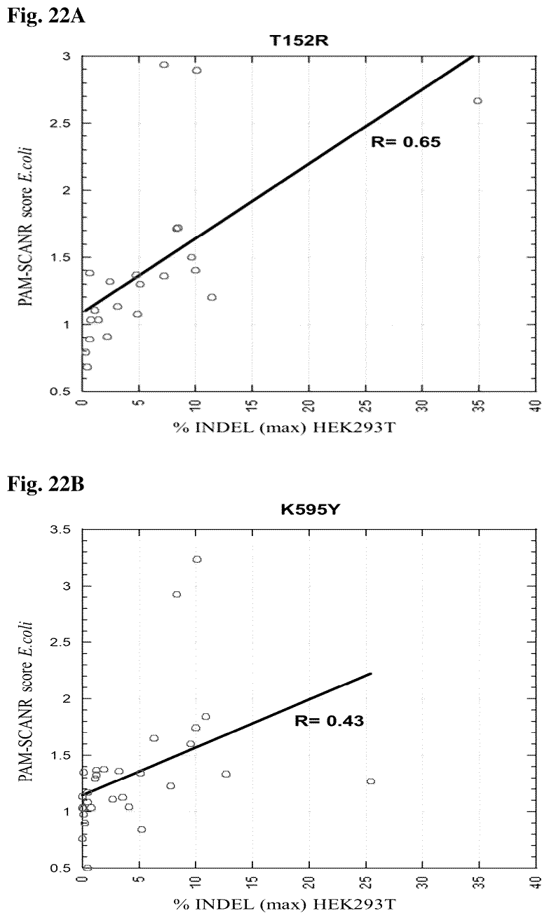

A correlation was observed between the maximal INDEL percentages observed and PAM-SCANR scores for T152R and K595Y (FIGS. 22A-22B). FIGS. 22A-22B shows the linear correlation between % INDEL (max) and the normalized bacterial PAM-SCANR score for LbCas12a-T152R (FIG. 22A) and LbCas12a-K595Y (FIG. 22B).

Notably, any point mutation with a PAM-SCANR score over 1.5 tested generated INDELs in 293T cells at rates greater than 5%. This suggests that any of the mutations tested in the PAM-SCANR experiment with a normalized score greater than 1.5 (rather than our stringent 1.67 cutoff) is likely to be able to generate INDELs at rates useful for most eukaryotic applications.

The foregoing is illustrative of the present invention, and is not to be construed as limiting thereof. The invention is defined by the following claims, with equivalents of the claims to be included therein.

1-25. (canceled)

26. A modified Lachnospiraceae bacterium CRISPR (Clustered Regularly Interspaced Short Palindromic Repeats) Cas12a (LbCas12a) polypeptide, wherein the modified LbCas12a polypeptide comprises an amino acid sequence having at least 95% identity to the amino acid sequence of SEQ ID NO:1 (LbCas12a) and a mutation located at one or more than one of the following positions of K116, E125, and/or Y616 with reference to position numbering of SEQ ID NO:1 and/or one or more of the following mutations of K120R, K120H, K120N, K120T, K120Y, K120Q, K121S, K121T, K121H, K121R, K121G, K121D, K121Q, D122R, D122K, D122H, D122E, D122N, T148H, T148S, T148A, T148C, T149A, T149C, T149S, T149G, T149H, T149P, T149F, T149N, T149D, T149V, T152W, T152Y, T152H, T152Q, T152E, T152L, T152F, D156Y, D156W, D156Q, D156H, D156I, D156V, D156L, D156E, E159K, E159R, E159H, E159Y, E159Q, Q529N, Q529T, Q529H, Q529A, Q529F, Q529G, Q529S, Q529P, Q529W, Q529D, G532D, G532N, G532S, G532H, G532F, G532K, G532Q, G532A, G532L, G532C, D535N, D535H, D535V, D535T, D535S, D535A, D535W, D535K, K538V, K538Q, K538W, K538Y, K538F, K538H, K538L, K538M, K538C, K538G, K538A, K538P, D541N, D541H, D541R, D541K, D541Y, D541I, D541A, D541S, D541E, Y542R, Y542K, Y542H, Y542Q, Y542F, Y542L, Y542M, Y542P, Y542V, Y542N, Y542T, L585G, L585H, L585F, K591W, K591F, K591Y, K591H, K591R, K591S, K591A, K591G, K591P, M592R, M592K, M592Q, M592E, M592A, K595R, K595Q, K595L, K595W, K595H, K595E, K595S, K595D, K595M, V596T, V596H, V596G, V596A, S599G, S599H, S599N, S599D, K600R, K600H, K600G, K601R, K601H, K601Q, K601T, Y646R, Y646E, Y646K, Y646H, Y646Q, Y646W, Y646N, W649H, W649K, W649Y, W649R, W649E, W649S, W649V, and/or W649T with reference to position numbering of SEQ ID NO:1, wherein the modified LbCas12a polypeptide exhibits a reduced PAM stringency and increased recognition of new protospacer adjacent motifs (PAMs) as compared to wild type LbCas12a, SEQ ID NO:1.

27. The modified LbCas12a polypeptide of claim 26, wherein the mutation located at position K116 with reference to position numbering of SEQ ID NO:1 is K116R or K116N, the mutation located at position E125 with reference to position numbering of SEQ ID NO:1 is E125R, E125K, E125Q, or E125Y, and the mutation located at position Y616 with reference to position numbering of SEQ ID NO:1 is Y616K, Y616R, Y616E, Y616F, or Y616H.

28. The modified LbCas12a polypeptide of claim 26, further comprising a mutation of T152R, T152K, D156R, D156K, G532R, and/or K538R with reference to position numbering of SEQ ID NO:1.

29. The modified LbCas12a polypeptide of claim 26, comprising a mutation of D156R and K538N, a mutation of D156R and Y542N, a mutation of D156R, K538N, and G532R, a mutation of D156R, G532R and Y542N, a mutation of D156R, K538N, and Y542N, and/or a mutation of D156R, G532R, K538N, and Y542N.

30. The modified LbCas12a polypeptide of claim 26, further comprising a mutation in the RuvC domain.

31. A fusion protein comprising the modified LbCas12a polypeptide of claim 26, and a polypeptide of interest.

32. The fusion protein of claim 31, wherein the polypeptide of interest comprises at least one polypeptide or protein domain having deaminase (deamination) activity, nickase activity, recombinase activity, transposase activity, methylase activity, glycosylase (DNA glycosylase) activity, glycosylase inhibitor activity (e.g., uracil-DNA glycosylase inhibitor (UGI)), demethylase activity, transcription activation activity, transcription repression activity, transcription release factor activity, histone modification activity, nuclease activity, single-strand RNA cleavage activity, double-strand RNA cleavage activity, restriction endonuclease activity (e.g., Fok1), nucleic acid binding activity, methyltransferase activity, DNA repair activity, DNA damage activity, dismutase activity, alkylation activity, depurination activity, oxidation activity, pyrimidine dimer forming activity, integrase activity, transposase activity, polymerase activity, ligase activity, helicase activity, and/or photolyase activity.

33. The fusion protein of claim 32, wherein the at least one polypeptide or protein domain having deaminase activity is a cytosine deaminase domain or an adenine deaminase domain.

34. The fusion protein of claim 32, wherein the at least one polypeptide has glycosylase inhibitor activity, optionally wherein the at least one polypeptide is a uracil-DNA glycosylase inhibitor (UGI).

35. A polynucleotide encoding the modified LbCas12a polypeptide of claim 26.

36. The polynucleotide of claim 35, wherein the polynucleotide encoding the modified LbCas12a polypeptide is operatively associated with a promoter, optionally wherein the promoter is a promoter region comprising an intron.

37. The polynucleotide of claim 35, wherein the polynucleotide is codon optimized for expression in an organism, optionally wherein the organism is an animal, a plant, a fungus, an archaeon, or a bacterium.

38. A complex comprising (a) the modified LbCas12a polypeptide of claim 26 and (b) a guide nucleic acid.

39. A nucleic acid construct encoding the complex of claim 38.

40. An expression cassette or vector comprising the polynucleotide of claim 35.

41. An expression cassette or vector comprising the complex of claim 38.

42. A cell comprising the polynucleotide of claim 35.

43. A method of modifying a target nucleic acid, comprising:
contacting the target nucleic acid with:
(a)(i) the modified LbCas12a polypeptide of claim 26, and (ii) a guide nucleic acid;
(b) a composition comprising (i) the modified LbCas12a polypeptide of claim 26, and (ii) a guide nucleic acid; and/or
(c) a Type V CRISPR-Cas system comprising:
a guide nucleic acid comprising a spacer sequence and a repeat sequence, and
(i) a fusion protein comprising the modified LbCas12a polypeptide of claim 26 and a polypeptide of interest, or
(ii) a nucleic acid encoding the modified LbCas12a polypeptide of claim 26 and a nucleic acid encoding a polypeptide of interest,
wherein the guide nucleic acid is capable of forming a complex with the modified LbCas12a polypeptide or the fusion protein and the spacer sequence is capable of hybridizing to a target nucleic acid, thereby guiding the modified LbCas12a polypeptide and the polypeptide of interest to the target nucleic acid, whereby the target nucleic acid is modified or modulated.

44. A method of editing a target nucleic acid, comprising:
contacting the target nucleic acid with:
(a)(i) a fusion protein comprising the modified LbCas12a polypeptide of claim 26 and a polypeptide of interest, and (a)(ii) a guide nucleic acid;
(b) a complex or a composition comprising a fusion protein comprising the modified LbCas12a polypeptide of claim 26 and a polypeptide of interest, and a guide nucleic acid; and/or
(c) a Type V CRISPR-Cas system comprising:
a guide nucleic acid comprising a spacer sequence and a repeat sequence, and
(i) a fusion protein comprising the modified LbCas12a polypeptide of claim 26 and a polypeptide of interest, or
(ii) a nucleic acid encoding the modified LbCas12a polypeptide of claim 26 and a nucleic acid encoding a polypeptide of interest,
wherein the guide nucleic acid is capable of forming a complex with the modified LbCas12a polypeptide or the fusion protein and the spacer sequence is capable of hybridizing to a target nucleic acid, thereby guiding the modified LbCas12a polypeptide and the polypeptide of interest to the target nucleic acid, thereby editing the target nucleic acid.


SEQUENCE LISTING (62 sequences)

SEQ ID NO: 1 - protein, 1228 aa, unidentified
  source 1..1228: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEDARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 4 - protein, 1238 aa, Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDIVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIDRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLRPVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 5 - protein, 1281 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLDKIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 6 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKVNYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 7 - protein, 1206 aa, unidentified
  source 1..1206: note = Lachnospiraceae bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSVIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFDTQASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGLYLQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 8 - protein, 1233 aa, unidentified
  source 1..1233: note = Lachnospiraceae sp.
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKKLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 9 - protein, 1227 aa, unidentified
  source 1..1227: note = Lachnospiraceae sp.
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIANINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIASNKEWLEYAQTSVKH

SEQ ID NO: 10 - protein, 1264 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYNKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNPLNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDISGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 11 - protein, 1373 aa, Moraxella bovoculi
MLFQDFTHLYPLSKTVRFELFIDRTLEHIHAKNFLSQDETMADMHQKVKVILDDYHRDFIADMMGEVKLTKLAEFYDVYLKFRKNPKDDELQKAQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFKDINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQKDFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIQASQAFFGKFDKICYNADKDYFEFHIDYAKFTDKAKNSRQIWTICSHGDKRYVYDKTANQNKGAAKGINVNDILKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 12 - protein, 1352 aa, unidentified
  source 1..1352: note = Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITESKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKIKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 13 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 14 - protein, 1324 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIDALKGFYIEDGFENNVLTPLATLLSSLDKYNLNGIFIRNNEALSSLSQNVYRNFSIDEAIDAQNAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNKDDLNLSISSTEWLDFVREKPYLK

SEQ ID NO: 15 - protein, 1484 aa, unidentified
  VARIANT 1073: note = Xaa can be any naturally occurring amino acid
  source 1..1484: note = Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAMIKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTWIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDXWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKKKRPAATKK

SEQ ID NO: 16 - protein, 1245 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSISEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQHGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 17 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYQWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 18 - protein, 166 aa, Escherichia coli
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 19 - protein, 166 aa, synthetic construct
  REGION 1..166: note = adenosine deaminase
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 20 - protein, 166 aa, synthetic construct
  REGION 1..166: note = adenosine deaminase
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 21 - protein, 166 aa, synthetic construct
  REGION 1..166: note = adenosine deaminase
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 22 - protein, 166 aa, synthetic construct
  REGION 1..166: note = adenosine deaminase
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 23 - protein, 228 aa, Rattus norvegicus
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 24 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 25 - DNA, 621 bp, Petromyzon marinus
  source 1..621: mol_type = genomic DNA
acagatgcagagtatgtgagaattcacgaaaagctggacatctataccttcaagaagcagttctttaacaataagaagtctgtgagccataggtgctacgtgctgttcgagctgaagagaaggggtgaaagaagggcatgtttttgggggtatgctgtgaacaagccccagtctggaactgagagaggcattcacgccgaaattttcagcatcagaaaggtggaggaatacctgagggataaccctggacagtttacaattaattggtattctagctggtctccatgcgctgactgtgccgagaagatcctggaatggtacaaccaggagctgagaggaaatggccataccctgaagatttgggcctgcaagctgtactatgaaaagaacgcaagaaatcagatcggactgtggaacctgagggataatggtgtggggctgaacgtgatggtgtccgagcactatcagtgctgtagaaagattttcattcagtcctcacataatcagctgaacgagaatagatggctggaaaagactctgaagagggctgagaagagaaggtccgaactgtcaattatgatccaggtgaagatcctgcacaccactaagtcacctgccgtg

SEQ ID NO: 26 - protein, 83 aa, unidentified
  source 1..83: note = Bacillus phage AR9
TNLSDIIEKETGKQLVIQESILMLPEEVEEVIGNKPESDILVHTAYDESTDENVMLLTSDAPEYKPWALVIQDSNGENKIKML

SEQ ID NO: 27-29 - skipped (3 entries)

SEQ ID NO: 30 - DNA, 23 bp, synthetic construct
  misc_feature 1..23: note = polynucleotide
  source 1..23: mol_type = other DNA
ggaatcccttctgcagcacctgg

SEQ ID NO: 31 - DNA, 23 bp, synthetic construct
  misc_feature 1..23: note = polynucleotide
  source 1..23: mol_type = other DNA
ctgatggtccatgtctgttactc

SEQ ID NO: 32 - DNA, 49 bp, synthetic construct
  misc_feature 1..49: note = polynucleotide
  misc_feature 7..11: note = wherein n is A, T, C or G
  source 1..49: mol_type = other DNA
cgatgtnnnnnggaatcccttctgcagcacctgggcgcaggtcacgagg

SEQ ID NO: 33 - DNA, 57 bp, synthetic construct
  misc_feature 1..57: note = polynucleotide
  misc_feature 43..46: note = n is a, c, g, or t
  misc_feature 47..51: note = wherein n is A, T, C or G
  source 1..57: mol_type = other DNA
aattcctcgtgacctgcgcccaggtgctgcagaagggattccnnnnnacatcgcatg

SEQ ID NO: 34 - DNA, 49 bp, synthetic construct
  misc_feature 1..49: note = polynucleotide
  misc_feature 7..11: note = wherein n is A, T, C or G
  source 1..49: mol_type = other DNA
cgatgtnnnnnctgatggtccatgtctgttactcgcgcaggtcacgagg

SEQ ID NO: 35 - DNA, 57 bp, synthetic construct
  misc_feature 1..57: note = polynucleotide
  misc_feature 43..46: note = n is a, c, g, or t
  misc_feature 47..51: note = wherein n is A, T, C or G
  source 1..57: mol_type = other DNA
aattcctcgtgacctgcgcgagtaacagacatggaccatcagnnnnnacatcgcatg

SEQ ID NO: 36 - DNA, 28 bp, synthetic construct
  misc_feature 1..28: note = polynucleotide
  misc_feature 1..5: note = wherein n is A, C, T or G
  source 1..28: mol_type = other DNA
nnnnnggaatcccttctgcagcacctgg

SEQ ID NO: 37 - DNA, 28 bp, synthetic construct
  misc_feature 1..28: note = polynucleotide
  misc_feature 1..5: note = wherein n is A, C, T or G
  source 1..28: mol_type = other DNA
nnnnnccttagggaagacgtcgtggacc

SEQ ID NO: 38 - RNA, 23 nt, synthetic construct
  misc_feature 1..23: note = polynucleotide
  source 1..23: mol_type = other RNA
ggaatcccttctgcagcacctgg

SEQ ID NO: 39 - DNA, 24 bp, synthetic construct
  misc_feature 1..24: note = polynucleotide
  misc_feature 1..5: note = wherein n is A, C, T or G
  source 1..24: mol_type = other DNA
nnnnnggaatcccttctgcagcac

SEQ ID NO: 40 - DNA, 1592 bp, Medicago truncatula
  source 1..1592: mol_type = genomic DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacttactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagaggtgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagg

SEQ ID NO: 41 - DNA, 2000 bp, Zea mays
  source 1..2000: mol_type = genomic DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttc

SEQ ID NO: 42 - protein, 160 aa, unidentified
FERNYDPRELRKETYLLYEIKWGKSGKLWRHWCQNNRTQHAEVYFLENIFNARRFNPSTHCSITWYLSWSPCAECSQKIVDFLKEHPNVLEIYVARLYYHEDERNRQGLRDLVNSGVTIRIMDLPDYNYCWKTFVSDQGGDEDYWPGHFAPWIKQYSLKL

SEQ ID NO: 43 - protein, 207 aa, synthetic construct
  REGION 1..207: note = evolved cytidine deaminase
TDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 44 - protein, 228 aa, synthetic construct
  REGION 1..228: note = evolved cytidine deaminase
SSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 45 - protein, 162 aa, synthetic construct
  REGION 1..162: note = evolved cytidine deaminase
SFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 46 - protein, 229 aa, Rattus norvegicus [sequence omitted]

SEQ ID NO: 47 - protein, 198 aa, Homo sapiens [sequence omitted]

SEQ ID NO: 48 - protein, 197 aa, synthetic construct
  REGION 1..197: note = cytosine deaminase
MDSLLMNRREFLYQFKNVRWAKGRRETYLCYVVKRRDSATSFSLDFGYLRNKNGCHVELLFLRYISDWDLDPGRCYRVTWFISWSPCYDCARHVADFLRGNPNLSLRIFTARLYFCEDRKAEPEGLRRLHRAGVQIAIMTFKDYFYCWNTFVENHGRTFKAWEGLHENSVRLSRQLRRILLPLYEVDDLRDAFRTCT

SEQ ID NO: 49 - DNA, 3987 bp, synthetic construct
  misc_feature 1..3987: note = polynucleotide
  source 1..3987: mol_type = other DNA
atggccgggagcaagaagcgccggataaagcaggacacgcagttcgagggcttcaccaacctgtaccaagtctccaagacgctccggttcgagcttatcccgcaagggaagaccctgaaacacatccaggaacaaggtttcatcgaggaggacaaggcccgcaacgaccactacaaggagctcaagcccataatcgatcggatctacaagacgtacgccgaccagtgcctccaactggtgcagctcgactgggagaacctgagcgccgccattgacagctaccgcaaggaaaagacggaggagacgcgcaacgcccttattgaggagcaagccacctaccgcaacgccatccacgactacttcatcgggcgcaccgacaacctgacggacgcgatcaacaagcgccacgcggaaatctacaagggccttttcaaggccgagctcttcaacgggaaggtcctaaaacagctcgggactgtcacgacaaccgagcatgagaacgccctccttcgcagcttcgacaagttcaccacatacttctcgggcttctaccggaaccgcaagaacgttttcagcgccgaggacatctccaccgccatcccgcacaggatcgtccaggacaacttccccaagttcaaggagaactgccacatcttcacgcgcctgattacagccgtaccttcacttcgtgagcacttcgagaacgtcaaaaaggccatcgggatcttcgtctccacgtccatcgaggaggtattctctttcccgttctataaccagctcctgacccagacgcagatcgacctctacaaccagctactgggcggcatcagccgggaggccgggaccgagaaaataaagggcctcaacgaagttctcaacctggccatccagaagaacgacgagaccgcgcatatcatcgcatccctgccgcatcgcttcattcctttgttcaagcagatattgagcgaccggaacaccctctcgttcatcctcgaagaattcaagagcgacgaggaggtcattcagtctttctgcaagtacaagacgctcctacggaatgagaatgtgctggagaccgcggaggcactcttcaatgagctgaactccattgacctgacccacatcttcattagccacaagaaactggagacgatctccagcgccctgtgcgaccactgggacactctccgcaacgccctctacgaacgccggatctccgaacttaccggcaagataactaagtcggctaaggagaaggtgcaacggagcctcaagcacgaggacatcaaccttcaggaaatcatctcagccgcgggcaaggagctgagcgaggcgtttaagcagaaaacatcggagatactgagccacgcgcacgcggccctggatcaaccgctgccgacgactctcaagaagcaagaggagaaggaaatccttaagtcccagctcgactcgctgctcggcctctatcacttgctcgactggttcgcggttgatgagtccaacgaggtggacccggagttctccgcgcgcctcacgggtattaagctggagatggagccaagcttaagcttctacaacaaggcccgcaactacgcgaccaaaaaaccgtactcagtcgagaaattcaagctgaatttccagatgcctacattggcgagggggtgggacgtgaaccgcgagaagaacaatggagccatcctgttcgtcaaaaatgggttgtactacctgggcatcatgcccaagcagaagggccgttacaaggccctgtcattcgagcctaccgagaagacctcggagggcttcgacaagatgtactacgactatttcccggacgccgccaagatgatcccgaagtgctccacgcagctcaaagccgtcacggcccacttccagacgcataccacgccgatacttctgagcaacaacttcattgagccgctagagatcacgaaggagatatacgacctaaacaaccccgaaaaggagcccaagaagttccagacagcctacgctaagaagacaggtgatcagaagggatatagggaggcactctgcaagtggatcgacttcacgcgcgacttcctgtcgaaatatacaaagacgaccagcattgacctaagttctctccgcccatcctcccagtacaaggatctgggcgagtattatgcggagctgaacccattgctgtaccacatcagcttccagaggatcgccgagaaggagattatggacgcggtggagacggggaaactatacctgttccaaatatataacaaggacttcgctaaagggcaccacgggaagcccaacctgcacacactctactggacgggcttgttttcgccagaaaatttggccaagacttcgatcaagctcaacggccaggcggagttgttttaccgtcccaagtctcgcatgaagcgcatggcgcatcgcctcggagagaaaatgcttaacaagaagctcaaggatcagaagacgcccatacctgatacgttgtaccaggaattgtacgactacgtgaaccaccgcctatcgcacgacctctcagacgaggcccgcgccctcctcccaaacgtgattactaaggaggtttcccatgaaataatcaaggaccgacggttcaccagcgacaaattttttttccacgtgcctatcacgctcaattaccaggcggccaactccccatcgaagttcaaccagcgcgtgaacgcctaccttaaggagcacccggagaccccaatcatcgggatcgaccgtggcgagcggaacctgatctatattacggtgatcgatagcaccgggaagatcctggagcagcgctccctgaacacaatccagcagtttgactaccagaagaaactcgacaaccgggagaaggagcgcgtcgcagcccggcaagcatggagtgtggtcggcaccataaaggacctgaaacagggttacctaagtcaagttatccacgagatcgttgacctgatgatacactatcaagccgtagtcgtgctggagaacctcaacttcgggtttaagtccaagcgcaccggcatcgcggagaaggcggtgtaccagcagttcgagaagatgctgatcgacaagctgaactgcctggtgctcaaggactaccctgcggagaaggtcggcggggtcttgaacccgtaccagctaaccgaccagttcacgagcttcgccaaaatgggcacgcagtccggattcttgttttatgtcccggctccatatacaagtaagatcgacccgctgacagggtttgttgacccattcgtgtggaagaccatcaagaaccacgagagcaggaaacacttcttagagggcttcgacttcctgcattacgacgttaagacaggcgacttcatcctgcacttcaagatgaaccgcaacctgtcgttccagaggggcctgcccggcttcatgcccgcctgggatatcgtctttgagaagaatgagacgcagttcgacgcgaaggggacgccgttcatcgctggaaagcggatcgtgccggtcatcgagaaccaccgcttcacgggtcgctaccgagatttataccccgccaacgaactaattgcgctgctggaggagaaggggatcgtgttccgagatggcagcaacattctcccgaagctgctggagaacgacgactcgcacgctattgacacgatggtcgccctcatacggagcgtgcttcagatgcggaacagtaacgctgccacgggcgaggactacattaactcccccgtccgcgacctcaacggggtctgcttcgatagccgcttccagaacccggagtggcctatggatgcggacgcgaacggggcctaccacatcgccctcaagggccaactcctgctcaaccacttgaaggaaagcaaagacctcaaattgcagaatggcatcagtaaccaggactggctcgcgtacatccaggaactgagaaacgggtccaagaagcggcgtatcaagcaagattga

SEQ ID NO: 50 - DNA, 3987 bp, synthetic construct
  misc_feature 1..3987: note = polynucleotide
  source 1..3987: mol_type = other DNA
atggcgggaagcaaaaagcgccggattaagcaagacacgcagttcgagggcttcacgaacctctaccaagtcagcaagaccctccggttcgagctgataccacagggaaagacgctcaagcacatccaggaacagggcttcatcgaggaggacaaggcgcgcaacgaccactacaaggagttgaaaccgatcatcgaccgcatctacaagacgtacgccgaccagtgcctccagctcgtgcagctcgactgggagaacctctccgccgccattgactcgtaccggaaggagaagactgaggagacccgcaacgccctgatcgaggagcaagcaacctaccggaacgccatccacgactacttcatcggccgcaccgacaacctcaccgacgcgatcaacaagcggcacgcggagatatacaaagggctgttcaaggcggagctgttcaacggcaaggtgctcaagcagctagggacggtgaccacgaccgagcacgagaacgcgctcctccgcagcttcgacaagttcaccacctacttcagcggcttctaccggaaccgcaagaatgtgttcagcgcggaggacatcagcacggccatcccgcaccgcatcgtccaggacaacttcccgaagttcaaggagaactgccacatcttcacccgcctgataaccgccgtcccctccctgcgggagcacttcgagaacgtcaaaaaggcaattgggatcttcgtctcgaccagcattgaggaggtgttcagcttccccttctacaaccagctcctcacccagacgcagatcgacctgtacaatcagttgctcggcgggataagccgcgaggcgggaaccgaaaaaatcaaggggctgaacgaagtgttgaacctcgccatccagaagaacgacgagaccgcgcacatcatcgcctccctgccccaccggttcatcccgctgttcaagcagatcctctctgaccggaacaccctgtccttcattcttgaggagttcaagtcggacgaggaggtcatccagagcttctgcaagtacaagacgctgctacggaacgagaacgtgctggagacggcggaggcactgttcaacgagctaaacagcatcgacctcacgcacatcttcatcagtcacaagaaactggagaccatctcctccgcgctgtgcgaccactgggacacgctcaggaacgcgctctacgagcgccgaatcagtgagctgacgggcaagatcacgaagtccgcgaaggagaaggtgcagcggtccctcaagcacgaggacatcaacctccaggagatcatctcagcggctgggaaagagctgtccgaggcgttcaagcagaaaacgagcgaaatcctgtcccacgcgcacgcggccctggatcagcctctgccgacgaccctcaagaaacaagaagaaaaggaaatcctcaagtcgcagctcgactcgctgctgggcctgtaccatctcctcgactggttcgccgtggacgagagcaacgaggtggaccccgagttctccgcgcggcttacggggatcaagctggagatggagcccagcctgtccttctacaacaaggcgcgcaactacgccaccaagaagccctacagcgtggagaagttcaagctcaacttccagatgcccactctcgcacgtgggtgggacgtcaaccgcgaaaaaaataatggggcgatcctgttcgtcaagaacggcctgtactacttgggcatcatgccgaaacagaagggccgctacaaggccctgagcttcgaaccgaccgagaaaacgagcgaggggttcgacaagatgtactacgactacttccccgacgccgcgaagatgattccaaagtgctccacgcagcttaaggccgtgacggcccacttccagacgcacacgaccccgatcctcctcagcaacaacttcatcgagcccctggagatcacgaaggagatatacgacctgaacaacccggagaaggagcccaagaaattccagaccgcctacgccaagaagacaggcgaccaaaagggttacagggaggccctctgcaagtggatcgacttcactagggacttcctgtccaagtacaccaagactacctctatcgacctgtccagcctccgcccgtcgtcccagtacaaagatttgggcgagtattacgcggagctgaacccactgctctaccacatcagcttccagcgcatcgcggagaaggagatcatggacgcagtggagacgggcaagctatacctatttcagatatacaacaaagacttcgctaagggacaccacggcaagcctaacctgcacaccctctactggacggggctcttcagcccggagaacctcgccaagacctcgatcaagctcaacggccaggccgagctgttctaccggcccaagtcccgcatgaagcggatggcccaccggctcggggagaaaatgctcaacaagaaattgaaggaccaaaaaacgccgatacccgacaccctataccaggagctgtacgactatgtgaaccaccgcctgagccacgacctcagcgacgaggcgcgggccctcctgccgaacgtcatcacaaaggaggtcagccacgagatcatcaaggaccggcgcttcacctccgacaagtttttctttcacgtgcccatcacgctcaactaccaggccgccaactcgccgtccaagttcaaccagcgcgtgaacgcctacctcaaggagcaccccgagaccccgatcatcgggattgaccgaggggagcggaacctcatctacatcaccgtcatcgacagcaccgggaagatccttgaacagcggtcgctcaacaccatccagcagttcgactaccagaagaaactcgacaaccgggagaaggagagagtggcggcccgccaggcttggtccgtcgtcgggacgattaaggacttgaaacaaggttacctgtcgcaagtgatccacgagatcgttgacctgatgatccactaccaagccgtcgtggtcctggagaacctcaacttcggcttcaagagcaaacgaaccggcatcgcggagaaggccgtgtaccagcagttcgaaaaaatgctgatcgacaagctgaactgcctcgtgctcaaggactaccccgctgagaaggtcggcggggtgctgaacccgtaccagctcactgaccagttcaccagcttcgcaaagatgggcacccagtccggcttcctgttctacgtgcctgcgccatacacctcgaagatcgacccgctcaccgggttcgtggaccccttcgtctggaagaccatcaagaaccacgagagccgcaagcacttcctggagggcttcgacttcctccactacgacgtcaagaccggggacttcatcctgcacttcaagatgaaccgcaacctcagtttccagcgcggcctgccggggttcatgcccgcttgggatatagtcttcgagaagaatgagacgcagttcgacgcgaagggcaccccgttcatcgccgggaagcgcatcgtgccggtcatcgagaaccaccggttcaccgggcgctaccgcgacctatacccggcgaacgagttgatcgccctcctggaggagaagggcatcgtgttccgcgacggctccaacatcctcccgaagctgctcgaaaacgacgactcccacgccatcgacacgatggtcgcgctgatccggtcggtgctccagatgcggaactccaacgccgcgacgggcgaggactacatcaacagtccggtccgcgatctgaacggcgtctgcttcgactcccggttccagaaccccgagtggccgatggacgcggacgcgaacggcgcataccacatcgccctaaaagggcaattgctgctcaaccacctcaaggaatccaaagacctaaagctccagaacggcatctccaaccaggactggctggcgtacatccaggaactgcggaacgggagcaaaaaacgtcggatcaagcaagattga

SEQ ID NO: 51 - DNA, 3987 bp, synthetic construct
  misc_feature 1..3987: note = polynucleotide
  source 1..3987: mol_type = other DNA
atggcgggctccaagaaacgccggattaagcaagatacccagttcgaggggttcacgaacctctaccaagtgagcaagaccctccgattcgaactgattcctcaggggaagaccctcaagcacatccaggagcaagggttcatcgaggaggacaaggcgcggaacgaccactacaaggaactcaaacccatcatcgaccgcatctacaagacctacgccgatcagtgcctccagctcgtgcagttggactgggagaacctcagcgcggccattgactcctaccggaaggagaaaacggaggagacgcgcaacgcgctcatcgaggaacaggcaacctatcgcaacgccatccacgactacttcatcgggaggactgacaacctcactgacgcgattaacaagcgccacgcggagatatacaagggactcttcaaagcggagctgtttaacggcaaggttctcaagcaactcggcactgtgaccacgaccgagcatgagaacgccctgctccgctccttcgacaagttcaccacctacttctccgggttctaccgcaaccgcaagaatgtcttcagcgcggaggacatcagcacggccattccacatcgaatcgtccaagataacttcccgaagttcaaggagaactgccacatcttcacccgactcattactgctgtaccgtcgttacgcgaacacttcgagaacgtcaagaaggcaattggaatcttcgtctctacgtcaatagaggaggtgttcagcttccctttctacaaccagctccttacgcagacccagatagacctgtacaatcagctcctcggtgggatcagccgggaggcggggactgagaagattaaagggctcaacgaggtcttgaacctggccatccaaaaaaacgatgagacggcgcacatcatcgcctcgctgccccaccggttcatcccgctgttcaagcagatcctcagtgacaggaacaccttgagctttatcctagaggagttcaagagcgacgaggaggtgatccagagcttctgcaagtacaaaaccctgctgaggaacgagaacgtcctggagacggcggaggcgctgttcaacgagctgaactctatcgacttaactcacatattcatctcgcacaagaagctggagactattagctctgcactctgcgaccactgggacaccctccgcaacgcgctctacgagcgccgcatctcggagctgaccgggaagatcaccaaatccgcgaaggaaaaggtccagcgttccctcaaacacgaggatattaacttacaggagattatctcagcggctgggaaggagttgtcagaggcgttcaagcagaaaacttccgagatcctgagccacgcgcacgcagcgctcgaccagcctctgcccaccaccctcaaaaagcaggaagaaaaagagatcctcaagagccagttggactccctgctggggctctatcaccttctcgactggttcgccgtcgatgagtcgaacgaggtggaccccgagttctccgcccggctgaccggcatcaagctagagatggagccgtccctcagcttctacaataaggcccgcaactacgcgaccaaaaaaccctacagcgtggagaagttcaagctgaacttccagatgccgaccttagcacgcggttgggacgtaaacagggagaagaacaatggagccatcctgttcgtcaagaacgggctttactacctcgggataatgcccaagcagaagggccgctacaaggccctttccttcgagccgacggagaaaacctccgaggggttcgacaagatgtactacgactacttccccgacgccgccaagatgatcccgaagtgctcaacgcagctaaaagccgtgaccgcccacttccagacccacacgacgccgatcctgctgagcaacaacttcatcgagccccttgagatcactaaggagatatacgacctgaacaaccccgagaaggagcccaagaagtttcaaaccgcctacgccaaaaaaactggcgaccaaaagggctacagggaggcgctgtgtaagtggatcgacttcacacgcgacttcctttcgaagtatacgaagacaacctctattgacctgagcagcctgcgtcctagctcccagtacaaagatttgggcgagtactacgcggagcttaatccactactctaccacatctcattccagcgcatcgctgagaaggaaatcatggacgcggtggagacaggcaaactgtacctcttccagatatacaacaaagacttcgctaaggggcaccacgggaagcccaaccttcatacgctctactggacgggcctattcagccccgaaaatctggccaagacctccatcaagctgaacggccaagcggagctgttctacagacccaagagccggatgaagcggatggcccacaggctcggcgagaaaatgcttaacaaaaagttgaaggaccagaaaacccctatccccgacaccctctaccaggaactgtacgactacgtgaaccacaggctctcgcacgacctttccgacgaggcccgtgccctactcccgaacgtcattaccaaagaggtttcgcacgagatcatcaaggaccggcggttcacgagcgacaagtttttctttcacgtccccatcacccttaactaccaggcggccaactccccatccaagttcaaccagcgtgtgaatgcctacctcaaggagcacccagagaccccgatcattgggatcgaccggggcgagcggaacctgatctacatcaccgtcatcgactcgacgggcaagattcttgagcagagatcgttgaataccatacagcagttcgactaccagaagaaactcgacaaccgcgagaaggagcgcgtggcggcccgccaggcgtggtccgtcgttgggacgattaaggacttgaaacaaggttatctgtcccaagtcatccacgagatcgttgatctgatgatccactatcaggcagtggtggtgctggagaatctcaacttcggcttcaagagtaagcggacgggaatcgccgagaaggccgtgtaccagcagttcgagaagatgctgatcgacaagctcaactgccttgtgctgaaagactacccggccgagaaggtcggcggcgtcctcaacccgtaccaacttaccgaccagttcacctccttcgccaagatgggcactcagtccgggttcttgttctacgtccccgcaccttacacctctaagatcgaccctctgactggcttcgtagatccattcgtgtggaagaccattaagaaccacgagagccgcaagcacttcctggagggcttcgacttcctgcactacgacgtgaagaccggggacttcatccttcacttcaagatgaaccggaacctcagcttccagcggggcctgccggggttcatgcccgcctgggacatcgtgttcgagaagaacgagacccagttcgacgcgaagggcacgcccttcatcgccgggaagcgtatcgtgccggtgatcgagaaccatcgtttcacgggtcgctaccgtgacctctacccggcgaacgagcttatcgcactcctggaggagaagggcatcgtcttccgggacggctccaacatcctcccgaaactgctggaaaacgacgactctcacgccatcgacacgatggtggccctcatccggtccgtgctccaaatgcggaacagcaacgccgccaccggtgaggactacatcaacagcccggtccgggatctgaacggggtgtgcttcgattcgcggttccagaatcctgagtggccgatggacgcggatgcaaacggggcgtaccacatcgcgctcaagggccagttacttctgaaccaccttaaggagtctaaagatttgaaactccagaacgggatctcgaaccaggactggctggcctacatccaagagttgcggaacggcagcaagaagcggcggattaagcaagattag

SEQ ID NO: 52 - DNA, 3790 bp, synthetic construct
  misc_feature 1..3790: note = expression cassette
  source 1..3790: mol_type = other DNA
ccatgggcagcaaactggaaaaatttacgaattgttatagcctgtccaagaccctgcgtttcaaagccatccccgttggcaaaacccaggagaatattgataataaacgtctgctggttgaggatgaaaaaagagcagaagactataagggagtcaaaaaactgctggatcggtactacctgagctttataaatgacgtgctgcatagcattaaactgaaaaatctgaataactatattagtctgttccgcaagaaaacccgaacagagaaagaaaataaagagctggaaaacctggagatcaatctgcgtaaagagatcgcaaaagcttttaaaggaaatgaaggttataaaagcctgttcaaaaaagacattattgaaaccatcctgccggaatttctggatgataaagacgagatagcgctcgtgaacagcttcaacgggttcacgaccgccttcacgggctttttcgataacagggaaaatatgttttcagaggaagccaaaagcacctcgatagcgttccgttgcattaatgaaaatttgacaagatatatcagcaacatggatattttcgagaaagttgatgcgatctttgacaaacatgaagtgcaggagattaaggaaaaaattctgaacagcgattatgatgttgaggattttttcgagggggaattttttaactttgtactgacacaggaaggtatagatgtgtataatgctattatcggcgggttcgttaccgaatccggcgagaaaattaagggtctgaatgagtacatcaatctgtataaccaaaagaccaaacagaaactgccaaaattcaaaccgctgtacaagcaagtcctgagcgatcgggaaagcttgagcttttacggtgaaggttataccagcgacgaggaggtactggaggtctttcgcaataccctgaacaagaacagcgaaattttcagctccattaaaaagctggagaaactgtttaagaattttgacgagtacagcagcgcaggtatttttgtgaagaacggacctgccataagcaccattagcaaggatatttttggagagtggaatgttatccgtgataaatggaacgcggaatatgatgacatacacctgaaaaagaaggctgtggtaactgagaaatatgaagacgatcgccgcaaaagctttaaaaaaatcggcagctttagcctggagcagctgcaggaatatgcggacgccgacctgagcgtggtcgagaaactgaaggaaattattatccaaaaagtggatgagatttacaaggtatatggtagcagcgaaaaactgtttgatgcggacttcgttctggaaaaaagcctgaaaaaaaatgatgctgttgttgcgatcatgaaagacctgctcgatagcgttaagagctttgaaaattacattaaagcattctttggcgagggcaaagaaacaaacagagacgaaagcttttatggcgacttcgtcctggcttatgacatcctgttgaaggtagatcatatatatgatgcaattcgtaattacgtaacccaaaagccgtacagcaaagataagttcaaactgtatttccagaacccgcagtttatgggtggctgggacaaagacaaggagacagactatcgcgccactattctgcgttacggcagcaagtactatctcgccatcatggacaaaaaatatgcaaagtgtctgcagaaaatcgataaagacgacgtgaacggaaattacgaaaagattaattataagctgctgccagggcccaacaagatgttaccgaaagtatttttttccaaaaaatggatggcatactataacccgagcgaggatatacagaagatttacaaaaatgggaccttcaaaaagggggatatgttcaatctgaatgactgccacaaactgatcgatttttttaaagatagcatcagccgttatcctaaatggtcaaacgcgtatgattttaatttctccgaaacggagaaatataaagacattgctggtttctatcgcgaagtcgaagaacagggttataaagttagctttgaatcggccagcaagaaagaggttgataaactggtggaggagggtaagctgtatatgtttcagatttataacaaagactttagcgacaaaagccacggtactcctaatctgcatacgatgtactttaaactgctgtttgatgagaataaccacggccaaatccgtctctccggtggagcagaactttttatgcggcgtgcgagcctaaaaaaggaagaactggtggtgcatcccgccaacagcccgattgctaacaaaaatccagataatcctaagaagaccaccacactgtcgtacgatgtctataaggataaacgtttctcggaagaccagtatgaattgcatataccgatagcaattaataaatgcccaaaaaacattttcaaaatcaacactgaagttcgtgtgctgctgaaacatgatgataatccgtatgtgatcggaattgaccgtggggagagaaatctgctgtatattgtagtcgttgatggcaagggcaacatcgttgagcagtatagcctgaatgaaataattaataattttaacggtatacgtattaaaaccgactatcatagcctgctggataaaaaggagaaagagcgttttgaggcacgccaaaattggacgagcatcgaaaacatcaaggaactgaaggcaggatatatcagccaagtagtccataaaatctgtgaactggtggagaagtacgacgctgtcattgccctggaagacctcaatagcggctttaaaaacagccgggtgaaggtggagaaacaggtataccaaaagtttgaaaagatgctcattgataagctgaactatatggttgataaaaagagcaacccgtgcgccactggcggtgcactgaaagggtaccaaattaccaataaatttgaaagctttaaaagcatgagcacgcagaatgggtttattttttatataccagcatggctgacgagcaagattgaccccagcactggttttgtcaatctgctgaaaaccaaatacacaagcattgcggatagcaaaaaatttatttcgagcttcgaccgtattatgtatgttccggaggaagatctgtttgaatttgccctggattataaaaacttcagccgcaccgatgcagattatatcaaaaaatggaagctgtacagttatggtaatcgtatacgtatcttccgtaatccgaagaaaaacaatgtgttcgattgggaagaggtctgtctgaccagcgcgtataaagaactgttcaacaagtacggaataaattatcagcaaggtgacattcgcgcactgctgtgtgaacagtcagataaagcattttatagcagctttatggcgctgatgagcctgatgctccagatgcgcaacagcataaccggtcgcacagatgttgactttctgatcagccctgtgaagaatagcgacggcatcttctacgattccaggaactatgaagcacaggaaaacgctattctgcctaaaaatgccgatgccaacggcgcctataatattgcacggaaggttctgtgggcgattggacagttcaagaaagcggaagatgagaagctggataaggtaaaaattgctattagcaataaggaatggctggagtacgcacagacatcggttaaacacggtagtaaaaggccggcggccacgaaaaaggccggccaggcaaaaaagaaaaagggagcggccgcactcgagcaccaccaccaccaccactgagcggccgc

SEQ ID NO: 53 - DNA, 8986 bp, synthetic construct
  misc_feature 1..8986: note = vector
  source 1..8986: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcaaactggaaaaatttacgaattgttatagcctgtccaagaccctgcgtttcaaagccatccccgttggcaaaacccaggagaatattgataataaacgtctgctggttgaggatgaaaaaagagcagaagactataagggagtcaaaaaactgctggatcggtactacctgagctttataaatgacgtgctgcatagcattaaactgaaaaatctgaataactatattagtctgttccgcaagaaaacccgaacagagaaagaaaataaagagctggaaaacctggagatcaatctgcgtaaagagatcgcaaaagcttttaaaggaaatgaaggttataaaagcctgttcaaaaaagacattattgaaaccatcctgccggaatttctggatgataaagacgagatagcgctcgtgaacagcttcaacgggttcacgaccgccttcacgggctttttcgataacagggaaaatatgttttcagaggaagccaaaagcacctcgatagcgttccgttgcattaatgaaaatttgacaagatatatcagcaacatggatattttcgagaaagttgatgcgatctttgacaaacatgaagtgcaggagattaaggaaaaaattctgaacagcgattatgatgttgaggattttttcgagggggaattttttaactttgtactgacacaggaaggtatagatgtgtataatgctattatcggcgggttcgttaccgaatccggcgagaaaattaagggtctgaatgagtacatcaatctgtataaccaaaagaccaaacagaaactgccaaaattcaaaccgctgtacaagcaagtcctgagcgatcgggaaagcttgagcttttacggtgaaggttataccagcgacgaggaggtactggaggtctttcgcaataccctgaacaagaacagcgaaattttcagctccattaaaaagctggagaaactgtttaagaattttgacgagtacagcagcgcaggtatttttgtgaagaacggacctgccataagcaccattagcaaggatatttttggagagtggaatgttatccgtgataaatggaacgcggaatatgatgacatacacctgaaaaagaaggctgtggtaactgagaaatatgaagacgatcgccgcaaaagctttaaaaaaatcggcagctttagcctggagcagctgcaggaatatgcggacgccgacctgagcgtggtcgagaaactgaaggaaattattatccaaaaagtggatgagatttacaaggtatatggtagcagcgaaaaactgtttgatgcggacttcgttctggaaaaaagcctgaaaaaaaatgatgctgttgttgcgatcatgaaagacctgctcgatagcgttaagagctttgaaaattacattaaagcattctttggcgagggcaaagaaacaaacagagacgaaagcttttatggcgacttcgtcctggcttatgacatcctgttgaaggtagatcatatatatgatgcaattcgtaattacgtaacccaaaagccgtacagcaaagataagttcaaactgtatttccagaacccgcagtttatgggtggctgggacaaagacaaggagacagactatcgcgccactattctgcgttacggcagcaagtactatctcgccatcatggacaaaaaatatgcaaagtgtctgcagaaaatcgataaagacgacgtgaacggaaattacgaaaagattaattataagctgctgccagggcccaacaagatgttaccgaaagtatttttttccaaaaaatggatggcatactataacccgagcgaggatatacagaagatttacaaaaatgggaccttcaaaaagggggatatgttcaatctgaatgactgccacaaactgatcgatttttttaaagatagcatcagccgttatcctaaatggtcaaacgcgtatgattttaatttctccgaaacggagaaatataaagacattgctggtttctatcgcgaagtcgaagaacagggttataaagttagctttgaatcggccagcaagaaagaggttgataaactggtggaggagggtaagctgtatatgtttcagatttataacaaagactttagcgacaaaagccacggtactcctaatctgcatacgatgtactttaaactgctgtttgatgagaataaccacggccaaatccgtctctccggtggagcagaactttttatgcggcgtgcgagcctaaaaaaggaagaactggtggtgcatcccgccaacagcccgattgctaacaaaaatccagataatcctaagaagaccaccacactgtcgtacgatgtctataaggataaacgtttctcggaagaccagtatgaattgcatataccgatagcaattaataaatgcccaaaaaacattttcaaaatcaacactgaagttcgtgtgctgctgaaacatgatgataatccgtatgtgatcggaattgaccgtggggagagaaatctgctgtatattgtagtcgttgatggcaagggcaacatcgttgagcagtatagcctgaatgaaataattaataattttaacggtatacgtattaaaaccgactatcatagcctgctggataaaaaggagaaagagcgttttgaggcacgccaaaattggacgagcatcgaaaacatcaaggaactgaaggcaggatatatcagccaagtagtccataaaatctgtgaactggtggagaagtacgacgctgtcattgccctggaagacctcaatagcggctttaaaaacagccgggtgaaggtggagaaacaggtataccaaaagtttgaaaagatgctcattgataagctgaactatatggttgataaaaagagcaacccgtgcgccactggcggtgcactgaaagggtaccaaattaccaataaatttgaaagctttaaaagcatgagcacgcagaatgggtttattttttatataccagcatggctgacgagcaagattgaccccagcactggttttgtcaatctgctgaaaaccaaatacacaagcattgcggatagcaaaaaatttatttcgagcttcgaccgtattatgtatgttccggaggaagatctgtttgaatttgccctggattataaaaacttcagccgcaccgatgcagattatatcaaaaaatggaagctgtacagttatggtaatcgtatacgtatcttccgtaatccgaagaaaaacaatgtgttcgattgggaagaggtctgtctgaccagcgcgtataaagaactgttcaacaagtacggaataaattatcagcaaggtgacattcgcgcactgctgtgtgaacagtcagataaagcattttatagcagctttatggcgctgatgagcctgatgctccagatgcgcaacagcataaccggtcgcacagatgttgactttctgatcagccctgtgaagaatagcgacggcatcttctacgattccaggaactatgaagcacaggaaaacgctattctgcctaaaaatgccgatgccaacggcgcctataatattgcacggaaggttctgtgggcgattggacagttcaagaaagcggaagatgagaagctggataaggtaaaaattgctattagcaataaggaatggctggagtacgcacagacatcggttaaacacggtagtaaaaggccggcggccacgaaaaaggccggccaggcaaaaaagaaaaagggagcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 54 - DNA, 23 bp, synthetic construct
  misc_feature 1..23: note = spacer sequence
  source 1..23: mol_type = other DNA
tcatctgtgcccctccctccctg

SEQ ID NO: 55 - DNA, 23 bp, synthetic construct
  misc_feature 1..23: note = spacer sequence
  source 1..23: mol_type = other DNA
gcggatgttccaatcagtacgca

SEQ ID NO: 56 - DNA, 23 bp, synthetic construct
  misc_feature 1..23: note = spacer sequence
  source 1..23: mol_type = other DNA
cagcccgctggccctgtaaagga

SEQ ID NO: 57 - DNA, 23 bp, synthetic construct
  misc_feature 1..23: note = spacer sequence
  source 1..23: mol_type = other DNA
tatgagttacaacgaacacctca

SEQ ID NO: 58 - DNA, 23 bp, synthetic construct
  misc_feature 1..23: note = spacer sequence
  source 1..23: mol_type = other DNA
cacgtctcatatgccccttggca

SEQ ID NO: 59 - DNA, 23 bp, synthetic construct
  misc_feature 1..23: note = spacer sequence
  source 1..23: mol_type = other DNA
gaacatgaaaacttaaatagaac

SEQ ID NO: 60 - DNA, 23 bp, synthetic construct
  misc_feature 1..23: note = spacer sequence
  source 1..23: mol_type = other DNA
gtcaggttggctgctgggctggc

SEQ ID NO: 61 - DNA, 23 bp, synthetic construct
  misc_feature 1..23: note = spacer sequence
  source 1..23: mol_type = other DNA
acagatggggctggacaattttt

SEQ ID NO: 62 - RNA, 43 nt, synthetic construct
  misc_feature 1..43: note = CRISPR RNA
  source 1..43: mol_type = other RNA
aatttctactaagtgtagatggaatcccttctgcagcacctgg